(19) United States
(12) Patent Application Publication
(10) Pub. No.: US 2014/0141008 A1
(43) Pub. Date: May 22, 2014

(54) STABLE HIGH PROTEIN CONCENTRATION FORMULATIONS OF HUMAN ANTI-TNF-ALPHA-ANTIBODIES

(71) Applicant: AbbVie Biotechnology Ltd., Hamilition (BM)

(72) Inventors: Wolfgang Fraunhofer, Gurnee, IL (US); Hans-Juergen Krause, Gruenstadt (DE); Michael Neu, Edingen-Neckarhausen (DE)

(21) Appl. No.: 14/170,061

(22) Filed: Jan. 31, 2014

Related U.S. Application Data
(63) Continuation of application No. 12/772,595, filed on May 3, 2010.
(60) Provisional application No. 61/175,380, filed on May 4, 2009.

(51) Int. Cl. C07K 16/24; A61K 47/12; A61K 47/22; A61K 47/26; A61K 9/00

(52) U.S. Cl. CPC C07K 16/241; A61K 47/26; A61K 9/0019; A61K 47/22; A61K 47/12
USPC 424/142.1

(57) ABSTRACT

The invention provides a liquid pharmaceutical formulation which does not include NaCl and comprises more than 20 mg of a polyol and at least about 100 mg/mL of a human anti-TNF-alpha antibody, or antigen-binding portion thereof. The invention provides a high concentration antibody formulation having long-term stability and advantageous characteristics for subcutaneous administration.

### RELATED APPLICATIONS

This application is a continuation of Ser. No. 12/772,595, filed on May 3, 2010, which claims priority to U.S. Provisional Application No. 61/175,380 filed on May 4, 2009, the entire contents of which are incorporated herein by this reference.

### BACKGROUND

The formulation of therapeutic proteins, such as antibodies, is often a challenge given the numerous desirable properties that the formulation must have to be economically and therapeutically successful, e.g., stability, suitability for administration, concentration. During manufacturing, storage, and delivery, therapeutic proteins have been known to undergo physical and chemical degradations. These instabilities can reduce the potency of the protein and increase the risk of adverse events in patients, and, therefore, significantly impact regulatory approval (see, e.g., Wang, et al. (2007) J Pharm Sci 96:1). As such, a stable protein formulation is essential to the success of a therapeutic protein.

To be effective, many therapeutic proteins require the administration of high doses, which, preferably, are formulated in high concentration formulations. High protein concentration formulations are desirable as they can impact the mode (e.g., intravenous vs. subcutaneous) and frequency of administration of the drug to a subject.

Despite the benefits of high protein concentration formulations, formulating high concentration therapeutic proteins presents numerous challenges. For example, increasing protein concentration often negatively impacts protein aggregation, solubility, stability, and viscosity (see, e.g., Shire, et al. (2004) J Pharm Sci 93:1390). Increased viscosity, which is a very common challenge for high protein solutions, can have negative ramifications on administration of the formulation, e.g., felt pain and burning syndromes and limitations in manufacturing, processing, fill-finish and drug delivery device options (see, e.g., Shire, et al. (2004) J Pharm Sci 93:1390). Even for therapeutic proteins having common structural features, e.g., antibodies, approved formulations to date have had varying ingredients and ranges of concentrations. For example, the anti-CD20 antibody Rituxan is formulated for intravenous administration at a concentration of 10 mg/mL, while the anti-RSV antibody Synagis is formulated for intramuscular administration at a concentration of 100 mg/mL. Thus, high protein formulations, especially antibody formulations, which can be used for therapeutic purposes remain a challenge. Accordingly, there is a need for stable, high concentration protein formulations that provide dosing and administrative advantages.

### SUMMARY OF THE INVENTION

The present invention is based, at least in part, on the discovery of new high-concentration formulations of human anti-TNF-α antibodies, or antigen-binding fragments thereof, e.g., adalimumab. The formulations of the invention provide a number of surprising characteristics given the high concentration of antibody. For example, the formulations of the invention maintain physical and chemical stability over extended periods despite the high concentration of protein, and have a viscosity suitable for subcutaneous administration. The formulations of the invention are established, at least in part, on the surprising finding that a human anti-TNF-alpha antibody, or antigen-binding portion thereof, can remain soluble at a high concentration (e.g., 100 mg/mL) and remain non-aggregated while maintaining a viscosity suitable for injection (e.g., subcutaneous administration). The formulation of the present invention is also surprising in that a high concentration (e.g., 100 mg/mL) of human anti-TNF-alpha antibody, or antigen-binding portion thereof, can remain soluble and remain non-aggregated and chemically stable (e.g., no oxidation or deamidation) over a wide pH range, e.g., about pH 5.2 to about pH 6.0. These beneficial characteristics are achieved without the need for NaCl as a stabilizer, and with an increase in a sugar alcohol excipient.

One aspect of the invention provides a liquid pharmaceutical formulation comprising more than 40 mg of a polyol and at least about 100 mg/mL of a human anti-TNF-alpha antibody, or antigen-binding portion thereof.

Another aspect of the invention provides a liquid pharmaceutical formulation comprising more than 20 mg of a polyol and at least about 100 mg/mL of a human anti-TNF-alpha antibody, or antigen-binding portion thereof. In one embodiment, the formulations of the invention do not contain NaCl.

The invention also features a liquid pharmaceutical formulation having a pH of about 5.0 to 6.4 and comprising at least about 100 mg/mL of a human anti-TNF-alpha antibody, or antigen-binding portion thereof, wherein the formulation does not contain NaCl and has a turbidity of less than 60 NTU after a standard 24 hour stir-stress assay or after 24 months of long-term storage as liquid.

The invention further provides a liquid pharmaceutical formulation having a pH of about 5.0 to 6.4 and comprising at least about 100 mg/mL of a human anti-TNF-alpha antibody, or antigen-binding portion thereof, wherein the formulation does not contain NaCl and has a turbidity of less than 100 NTU after a standard 48 hour stir-stress assay.

Another aspect of the invention includes a liquid pharmaceutical formulation having a pH of about 5.0 to 6.4 and comprising at least about 100 mg/mL of a human anti-TNF-alpha antibody, or antigen-binding portion thereof, wherein the formulation does not contain NaCl and has a turbidity of less than 40 NTU after 3 months storage at 5° C., 25° C., or 40° C.

The invention also provides a liquid pharmaceutical formulation comprising at least about 100 mg/mL of a human anti-TNF-alpha antibody, or antigen-binding portion thereof; more than about 20 mg/mL of a polyol; 0.1-2.0 mg/mL of a surfactant; about 1.15-1.45 mg/mL of citric acid*H₂O; about 0.2-0.4 mg/mL of sodium citrate dehydrate; about 1.35-1.75 mg/mL of Na₂HPO₄*2 H₂O; about 0.75-0.95 mg/mL of NaH₂PO₄*2 H₂O, wherein the formulation has a pH of about 4.7 to 6.5 and does not comprise NaCl.

The formulation of the invention is suitable for subcutaneous administration. As such, the invention also includes the use of the formulation of the invention comprising a human TNF alpha antibody, or antigen-binding portion thereof, for the treatment of a disorder associated with detrimental TNF alpha activity in a subject.

In one embodiment, the formulation of the invention has a concentration of a human TNF alpha antibody, or antigen binding portion thereof, and a viscosity of between about 3.1-3.3 mPas*s.

In one embodiment, the formulation of the invention comprises more than 20 mg of a polyol. Additional amounts of polyol which may be included in the formulation of the invention are more than 30 mg of the polyol. Alternatively, more than 40 mg of the polyol may be used in the formulation of the invention, including, but not limited to, 40-45 mg, or about 42 mg.

In one embodiment, the polyol used in the formulation of the invention is a sugar alcohol, such as, but not limited to, mannitol or sorbitol. In one embodiment, the formulation comprises about 40-45 mg/mL of either mannitol or sorbitol.

Various surfactants known in the art may be used in the formulation of the invention. In one embodiment, the surfactant is polysorbate 80. In a further embodiment, about 0.1-2.0 mg/mL of polysorbate 80 is used in the formulation of the invention.

In one embodiment of the invention, the formulation comprises about 1.30-1.31 mg/mL of citric acid*H₂O.

In another embodiment of the invention, the formulation comprises about 0.30-0.31 mg/mL sodium citrate dehydrate.

In still another embodiment of the invention, the formulation comprises about 1.50-1.56 mg/mL of Na₂HPO₄*2 H₂O.

In a further embodiment of the invention, the formulation comprises about 0.83-0.89 mg/mL of NaH₂PO₄*2 H₂O.

In another embodiment, the pH of the formulation of the invention ranges from about 4.8 to about 6.4. For example, the pH of the formulation of the invention may range from either about 5.0 to about 5.4 (e.g., about 5.2) or may range from about 5.8 to about 6.4 (e.g., about 6.0).

An advantage of the formulation of the invention is that it provides a high concentration of antibody without increased protein aggregation, which commonly occurs with increased protein concentration. In one embodiment, the formulation of the invention has less than about 1% aggregate protein.

Also contemplated as part of the invention are formulations described herein having a concentration of at least about 50 mg/mL of a human anti-TNF alpha antibody, or antigen-binding portion thereof.

In one embodiment, the human antibody, or antigen-binding portion thereof, comprises a light chain comprising a CDR3 domain comprising an amino acid sequence set forth as SEQ ID NO: 3 and a heavy chain comprising a CDR3 domain comprising an amino acid sequence set forth as SEQ ID NO: 4.

In one embodiment of the invention, the antibody has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9 and has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

The antibody of the invention may have certain functional characteristics. For example, the human antibody, or an antigen-binding portion thereof, may dissociate from human TNFα with a K_{d} of 1×10⁻⁸ M or less, dissociate from human TNFα with a K_{off} rate constant of 1×10⁻³ s⁻¹ or less, both determined by surface plasmon resonance, and/or neutralize human TNFα cytotoxicity in a standard in vitro L929 assay with an IC₅₀ of 1×10⁻⁷ M or less.

In one embodiment, the human antibody, or antigen-binding portion thereof, is a human IgG1 kappa antibody.

In one embodiment of the invention, the light chain of the human antibody, or antigen-binding portion thereof, further comprises a CDR2 domain comprising an amino acid sequence set forth as SEQ ID NO: 5 and a CDR1 domain comprising an amino acid sequence set forth as SEQ ID NO: 7, and/or the heavy chain of the human antibody comprises a CDR2 domain comprising an amino acid sequence set forth as SEQ ID NO: 6 and a CDR1 domain comprising an amino acid sequence set forth as SEQ ID NO: 8. In another embodiment, the light chain of the human antibody, or antigen-binding portion thereof, comprises the amino acid sequence set forth as SEQ ID NO: 1 and the heavy chain of the human antibody comprises the amino acid sequence set forth as SEQ ID NO: 2. Also included in the invention are human antibodies, or antigen-binding portions thereof, having amino acid sequences which are at least 80%, 85%, 90%, 95%, 96%, 97%, 98%, or 99% identical to the SEQ ID NOs recited herein.

In yet another embodiment of the invention, the human antibody, or antigen-binding portion thereof, is adalimumab.

### BRIEF DESCRIPTION OF THE DRAWINGS

FIG. 1 is a graph depicting the presence of high molecular weight (hmw) protein specimen in a solution containing 0.1% Solutol. According to MALS (grey line), aggregate molar masses equal up to nearly 10⁹ g/mol, accounting for 2.6% of total protein (UV280, black line). Storage at 40° C. for 12 w.

FIGS. 2A and 2B are graphs depicting the early-stage detection of high molecular weight (hmw) aggregates emerging during 40° C. storage. Whereas no aggregates could be detected via UV280 (black curve), MALS (grey curve) unambiguously proved the presence of hmw specimen. One week storage (A) versus original sample (B).

FIG. 3 is a graph depicting the turbidity vs. freeze/thaw cycles of formulations F1-F6.

FIG. 4 is a graph depicting the polydispersity index vs. freeze/thaw cycles of formulations F1-F6.

FIG. 5 is a graph depicting the aggregate levels by SEC vs. freeze/thaw cycles of formulations F1-F6.

FIG. 6 is a graph depicting Tm in ° C. by DSC of formulations F1-F6 at T0.

FIG. 7 is a graph depicting aggregate levels by SEC vs. stirring time of formulations F1-F6.

FIG. 8 is a graph depicting the comparison of turbidity values obtained in stability studies after 3 months storage of F2, F6 and F7 (3 representative batches 01032-0134).

FIG. 9 is a graph depicting the comparison of visible particle values by DAC score obtained in stability studies after 3 months storage of F2, F6 and F7 (3 representative batches 01032-0134).

FIG. 10 is a graph depicting the comparison of sub-visible particle values (>=10 μm) obtained in stability studies after 3 months storage of F2, F6 and F7 (3 representative batches 01032-0134).

FIG. 11 is a graph depicting the comparison of sub-visible particle values (>=25 μm) obtained in stability studies after 3 months storage of F2, F6 and F7 (3 representative batches 01032-0134).

FIG. 12 is a graph depicting the comparison of residual monomer content obtained in stability studies after 3 months storage of F2, F6 and F7 (3 representative batches 01032-0134).

FIG. 13 is a graph depicting the comparison of sum of lysine variants obtained in stability studies after 3 months storage of F2, F6 and F7 (3 representative batches 01032-0134).

FIG. 14 is a graph depicting the turbidity data comparing F2, F6 and F7 in terms of stability against stir stress at different stirring speeds after 24 hours.

FIG. 15 is a graph depicting the DLS data (Z-average values) comparing F2, F6 and F7 in terms of stability against stir stress at different stirring speeds after 24 hours.

FIG. 16 is a graph depicting turbidity data comparing F2, F6 and F7 in terms of stability against stress before and after several pump cycles.

FIG. 17 is a graph depicting DLS data (Z-average) comparing F2, F6 and F7 in terms of stability before and after several pump cycles.

FIG. 18 is a graph depicting SEC data (aggregate levels) comparing F2, F6 and F7 in terms of stability before and after several pump cycles.

FIG. 19 is a graph depicting the visual score of 100 mg/mL formulations filled using a peristaltic pump.

FIG. 20 is a graph depicting the visual score of 100 mg/mL formulations filled using a piston pump.

FIG. 21 is a graph depicting the turbidity of 100 mg/mL formulations filled using a peristaltic pump.

FIG. 22 is a graph depicting the turbidity of 100 mg/mL formulations filled using a piston pump.

FIG. 23 is a graph depicting the turbidity at T0 and after 4 weeks storage at 5° C. of formulations F8-F11.

FIG. 24 is a graph depicting the monomer content at T0 and after 4 weeks storage at 5° C. of formulations F8-F11.

FIG. 25 is a graph depicting the aggregate levels at T0 and after 4 weeks storage at 5° C. of formulations F8-F11.

FIG. 26 is a graph depicting the subvisible particle count at T0 and after 4 weeks storage at 5° C. of formulations F8-F11.

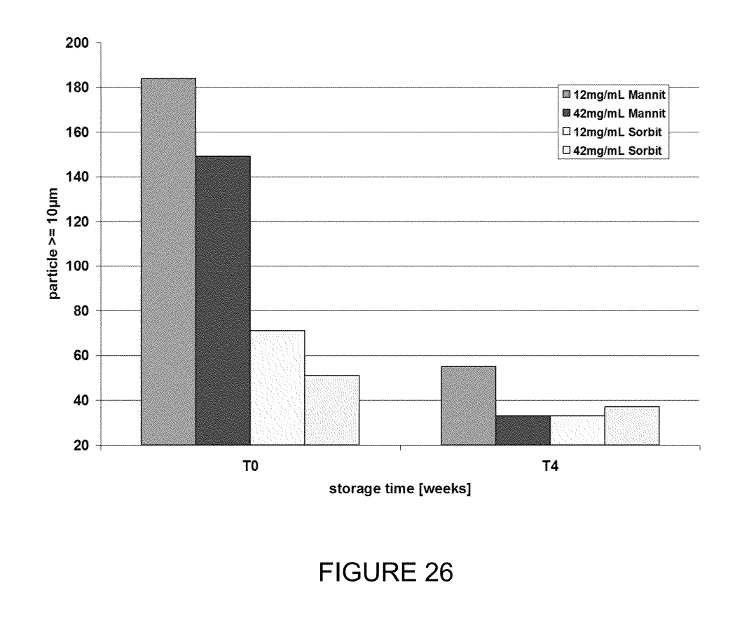

### DETAILED DESCRIPTION OF THE INVENTION

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The term “pharmaceutical formulation” refers to preparations which are in such form as to permit the biological activity of the active ingredients to be unequivocally effective, and which contain no additional components which are significantly toxic to the subjects to which the formulation would be administered.

The phrase “pharmaceutically acceptable carrier” is art recognized and includes a pharmaceutically acceptable material, composition or vehicle, suitable for administration to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to or impacting safety of the patient.

“Pharmaceutically acceptable excipients” (vehicles, additives) are those which can reasonably be administered to a subject mammal to provide an effective dose of the active ingredient employed.

The term “excipient” refers to an agent which may be added to a formulation to provide a desired consistency, e.g., altering the bulk properties, to improve stability, and/or to adjust osmolality. Examples of commonly used excipients include, but are not limited to, sugars, polyols, amino acids, surfactants, and polymers.

A commonly used excipient is a polyol. As used herein, a “polyol” is a substance with multiple hydroxyl groups, and includes sugars (reducing and nonreducing sugars), sugar alcohols and sugar acids. Preferred polyols herein have a molecular weight which is less than about 600 kD (e.g., in the range from about 120 to about 400 kD). Non-limiting examples of polyols are fructose, mannose, maltose, lactose, arabinose, xylose, ribose, rhamnose, galactose, glucose, sucrose, trehalose, sorbose, melezitose, raffinose, mannitol, xylitol, erythritol, threitol, sorbitol, glycerol, L-gluconate and metallic salts thereof.

As used herein, “buffer” refers to a buffered solution that resists changes in pH by the action of its acid-base conjugate components. The buffers of this invention have a pH in the range from about 4 to about 8; preferably from about 4.5 to about 7; and most preferably has a pH in the range from about 5.0 to about 6.5. Examples of buffers that will control the pH in this range include phosphate, acetate (e.g., sodium acetate), succinate (such as sodium succinate), gluconate, glutamate, histidine, citrate and other organic acid buffers. In one embodiment, a buffer suitable for use in the formulations of the invention is a citrate and phosphate buffer.

The term “surfactant” generally includes those agents which protect a protein in a formulation from air/solution interface-induced stresses and solution/surface induced-stresses. For example, a surfactant may protect the protein from aggregation. Suitable surfactants may include, e.g., polysorbates, polyoxyethylene alkyl ethers such as Brij 35®, or poloxamer such as Tween 20, Tween 80, or poloxamer 188. Preferred detergents are poloxamers, e.g., Poloxamer 188, Poloxamer 407; polyoxyethylene alkyl ethers, e.g., Brij 35®, Cremophor A25, Sympatens ALM/230; and polysorbates/Tweens, e.g., Polysorbate 20, Polysorbate 80, Mirj, and Poloxamers, e.g., Poloxamer 188, and Tweens, e.g., Tween 20 and Tween 80.

A “stable” formulation is one in which the antibody therein essentially retains its physical stability and/or chemical stability and/or biological activity during the manufacturing process and/or upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. (1993) Adv. Drug Delivery Rev. 10: 29-90. For example, in one embodiment, the stability of the protein is determined according to the percentage of monomer protein in the solution, with a low percentage of degraded (e.g., fragmented) and/or aggregated protein. Preferably, the formulation is stable at room temperature (about 30° C.) or at 40° C. for at least 1 month and/or stable at about 2-8° C. for at least 1 year or for at least 2 years. Furthermore, the formulation is preferably stable following freezing (to, e.g., −70° C.) and thawing of the formulation, hereinafter referred to as a “freeze/thaw cycle.”

An antibody “retains its physical stability” in a pharmaceutical formulation if it shows substantially no signs of, e.g., aggregation, precipitation and/or denaturation upon visual examination of color and/or clarity, or as measured by UV light scattering or by size exclusion chromatography. Aggregation is a process whereby individual molecules or complexes associate covalently or non-covalently to form aggregates. Aggregation can proceed to the extent that a visible precipitate is formed.

Stability, such as physical stability of a formulation, may be assessed by methods well-known in the art, including measurement of a sample's apparent attenuation of light (absorbance, or optical density). Such a measurement of light attenuation relates to the turbidity of a formulation. The turbidity of a formulation is partially an intrinsic property of the protein dissolved in solution and is commonly determined by nephelometry, and measured in Nephelometric Turbidity Units (NTU).

The degree of turbidity, e.g., as a function of the concentration of one or more of the components in the solution, e.g., protein and/or salt concentration, is also referred to as the “opalescence” or “opalescent appearance” of a formulation. The degree of turbidity can be calculated by reference to a standard curve generated using suspensions of known turbidity. Reference standards for determining the degree of turbidity for pharmaceutical compositions can be based on the European Pharmacopeia criteria (European Pharmacopoeia, Fourth Ed., Directorate for the Quality of Medicine of the Council of Europe (EDQM), Strasbourg, France). According to the European Pharmacopeia criteria, a clear solution is defined as one with a turbidity less than or equal to a reference suspension which has a turbidity of approximately 3 according to European Pharmacopeia standards. Nephelometric turbidity measurements can detect Rayleigh scatter, which typically changes linearly with concentration, in the absence of association or nonideality effects. Other methods for assessing physical stability are well-known in the art.

An antibody “retains its chemical stability” in a pharmaceutical formulation, if the chemical stability at a given time is such that the antibody is considered to still retain its biological activity as defined below. Chemical stability can be assessed by, e.g., detecting and quantifying chemically altered forms of the antibody. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation or oxidation) which can be evaluated by ion-exchange chromatography, for example.

An antibody “retains its biological activity” in a pharmaceutical formulation, if the antibody in a pharmaceutical formulation is biologically active for its intended purpose. For example, biological activity is retained if the biological activity of the antibody in the pharmaceutical formulation is within about 30%, about 20%, or about 10% (within the errors of the assay) of the biological activity exhibited at the time the pharmaceutical formulation was prepared (e.g., as determined in an antigen binding assay).

In a pharmacological sense, in the context of the present invention, a “therapeutically effective amount” or “effective amount” of an antibody refers to an amount effective in the prevention or treatment or alleviation of a symptom of a disorder for the treatment of which the antibody is effective. A “disorder” is any condition that would benefit from treatment with the antibody. This includes chronic and acute disorders or diseases including those pathological conditions which predisposes the subject to the disorder in question.

“Treatment” refers to both therapeutic treatment and prophylactic or preventative measures. Those in need of treatment include those already with the disorder as well as those in which the disorder is to be prevented.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intriacranial, intraarticular, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The term “human TNF-alpha” (abbreviated herein as hTNF-alpha, TNFα, or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of hTNF-alpha is described further in, for example, Pennica, D., et al. (1984) Nature 312:724-729; Davis, J. M., et al. (1987) Biochem 26:1322-1326; and Jones, E. Y., et al. (1989) Nature 338:225-228. The term human TNF-alpha is intended to include recombinant human TNF-alpha (rhTNF-alpha), which can be prepared by standard recombinant expression methods or purchased commercially (R & D Systems, Catalog No. 210-TA, Minneapolis, Minn.).

The term “antibody”, as used herein, is intended to refer to immunoglobulin molecules comprised of four polypeptide chains, two heavy (H) chains and two light (L) chains inter-connected by disulfide bonds. Other naturally occurring antibodies of altered structure, such as, for example, camelid antibodies, are also included in this definition. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In one embodiment of the invention, the formulation contains an antibody with CDR1, CDR2, and CDR3 sequences like those described in U.S. Pat. Nos. 6,090,382 and 6,258,562, each incorporated by reference herein.

As used herein, the term “CDR” refers to the complementarity determining region within a antibody variable sequence. There are three CDRs in each of the variable regions of the heavy chain and the light chain, which are designated CDR1, CDR2 and CDR3, for each of the variable regions. The exact boundaries of these CDRs have been defined differently according to different systems. The system described by Kabat (Id.) not only provides an unambiguous residue numbering system applicable to any variable region of an antibody, but also provides precise residue boundaries defining the three CDRs. These CDRs may be referred to as Kabat CDRs. Chothia et al. found that certain sub-portions within Kabat CDRs adopt nearly identical peptide backbone conformations, despite having great diversity at the level of amino acid sequence (Chothia et al. (1987) Mol. Biol. 196:901-917; Chothia et al. (1989) Nature 342:877-883) These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (1995) FASEB J. 9:133-139 and MacCallum (1996) J. Mol. Biol. 262(5):732-45. Still other CDR boundary definitions may not strictly follow one of the herein described systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The term “antigen-binding portion” of an antibody (or simply “antibody portion”), as used herein, refers to one or more fragments of an antibody that retain the ability to specifically bind to an antigen (e.g., hTNF-alpha). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). In one embodiment of the invention, the formulation contains an antigen-binding portions described in U.S. Pat. Nos. 6,090,382 and 6,258,562, each incorporated by reference herein.

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

The term “human antibody”, as used herein, is intended to include antibodies having variable and constant regions derived from human germline immunoglobulin sequences. The human antibodies used in the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further in Section II, below), antibodies isolated from a recombinant, combinatorial human antibody library (described further in Section III, below), antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

An “isolated antibody”, as used herein, is intended to refer to an antibody that is substantially free of other antibodies having different antigenic specificities (e.g., an isolated antibody that specifically binds hTNF-alpha is substantially free of antibodies that specifically bind antigens other than hTNF-alpha). An isolated antibody that specifically binds hTNF-alpha may, however, have cross-reactivity to other antigens, such as TNF-alpha molecules from other species. Moreover, an isolated antibody may be substantially free of other cellular material and/or chemicals.

A “neutralizing antibody”, as used herein (or an “antibody that neutralized hTNF-alpha activity”), is intended to refer to an antibody whose binding to hTNF-alpha results in inhibition of the biological activity of hTNF-alpha. This inhibition of the biological activity of hTNF-alpha can be assessed by measuring one or more indicators of hTNF-alpha biological activity, such as hTNF-alpha-induced cytotoxicity (either in vitro or in vivo), hTNF-alpha-induced cellular activation and hTNF-alpha binding to hTNF-alpha receptors. These indicators of hTNF-alpha biological activity can be assessed by one or more of several standard in vitro or in vivo assays known in the art, and described in U.S. Pat. Nos. 6,090,382 and 6,258,562, each incorporated by reference herein. Preferably, the ability of an antibody to neutralize hTNF-alpha activity is assessed by inhibition of hTNF-alpha-induced cytotoxicity of L929 cells. As an additional or alternative parameter of hTNF-alpha activity, the ability of an antibody to inhibit hTNF-alpha-induced expression of ELAM-1 on HUVEC, as a measure of hTNF-alpha-induced cellular activation, can be assessed.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “Kₒₙ”, as used herein, is intended to refer to the on rate constant for association of a binding protein (e.g., an antibody) to the antigen to form the, e.g., antibody/antigen complex as is known in the art.

The term “K_{off}”, as used herein, is intended to refer to the off rate constant for dissociation of an antibody from the antibody/antigen complex.

The term “Kd”, as used herein, is intended to refer to the dissociation constant of a particular antibody-antigen interaction and refers to the value obtained in a titration measurement at equilibrium, or by dividing the dissociation rate constant (k_{off}) by the association rate constant (kₒₙ).

Various aspects of the invention are described in further detail in the following subsections.

### II. Formulations of the Invention

The present invention features liquid pharmaceutical formulations (e.g., antibody formulations) having improved properties as compared to art-recognized formulations. The present invention is based on the surprising finding that by removing NaCl and adding more than 20 mg/mL of a polyol, e.g., a sugar alcohol, the concentration of a human TNF alpha antibody in a formulation can be increased to about 100 mg/mL. Despite the high concentration of antibody, the formulation of the invention is able to maintain solubility and stability of the protein, e.g., during manufacturing, storage, and/or repeated freeze/thaw processing steps or extended exposure to increased air-liquid interfaces. In addition, the formulation of the invention maintains a low level of protein aggregation (i.e., less than 1%), despite having about 100 mg/mL of antibody. The formulation of the invention also, surprisingly, maintain a low viscosity within ranges suitable for subcutaneous injection, despite having about 100 mg/mL of antibody. Furthermore, the formulation of the invention, e.g., high concentration TNF alpha antibody, maintains solubility, maintains a low viscosity suitable for subcutaneous injection, and maintains stability over a pH range of almost one, e.g., pH 5.2 to pH 6.0. In one embodiment, turbidity of the formulation is less than 100 NTU after a standard 48 hour stir-stress assay. Thus, the high antibody formulation of the invention overcomes a number of known challenges for formulations, including stability, viscosity, turbidity, and physical degradation challenges.

A surprising feature of the formulation of the invention is that in the absence of NaCl, the overall viscosity of the formulation remains low (e.g., about 3.1-3.3 mPas*s, e.g., about 3.00, 3.05, 3.10, 3.15, 3.20, 3.25, 3.30, 3.35, or about 3.40 mPas*s), while the antibody concentration is high (e.g., 100 mg/mL or greater). Generally, viscosity increases as the protein concentration increases (see Shire et al. (2004) J Pharm Sci 93:1390 for review). Such an increase is almost always counteracted by adding ionic excipients, e.g., NaCl and MgCl₂, however, the addition of such excipients may also result in increased turbidity of the solution. Increased turbidity is often associated with the formation of insoluble protein aggregates, precipiates, or protein particles (e.g., aggregation). Thus, the liquid pharmaceutical formulation of the invention provides a high antibody concentration (e.g., at least 100 mg/mL) with a viscosity suitable for subcutaneous administration, without the need for the addition of NaCl.

In one embodiment, formulations of the invention include high concentrations of proteins such that the liquid formulation does not show significant opalescence, aggregation, or precipitation.

In another embodiment, formulations of the invention include high concentrations of proteins such that are suitable for, e.g., subcutaneous administration without significant felt pain (e.g., as determined by a visual analog scale (VAS) score).

The formulations of the invention comprise a high protein concentration, including, for example, a protein concentration about 50 mg/mL or about 100 mg/mL of a human anti-TNF-alpha antibody or antigen-binding fragment thereof. Accordingly, as described in Example 1 below, in one aspect of the invention the liquid pharmaceutical formulation comprises a human anti-TNF alpha antibody concentration of about 50 mg/mL. As described in Examples 2-6 below, in another aspect of the invention the liquid pharmaceutical formulation comprises a human anti-TNF alpha antibody concentration of about 100 mg/mL. In yet another aspect of the invention the liquid pharmaceutical formulation comprises a human anti-TNF alpha antibody concentration of about 150 mg/mL. Although the preferred embodiments of the invention are formulations comprising high protein concentrations, it is also contemplated that the formulations of the invention may comprise an antibody concentration between about 1 mg/mL and about 150 mg/mL or about 40 mg/mL-125 mg/mL. Concentrations and ranges intermediate to the above recited concentrations are also intended to be part of this invention (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 mg/mL).

In another aspect, the invention provides a liquid pharmaceutical composition comprising a polyol, a surfactant, and a buffer system, in amounts sufficient to formulate an antibody, e.g., adalimumab, for therapeutic use at a concentration of greater than about, for example, 100 mg/mL. In one embodiment, the liquid pharmaceutical compositions do not comprise NaCl.

It should be noted, however, that although the preferred formulations of the invention do not comprise NaCl, a small amount of NaCl may be present in the formulations, e.g., from about 0.01 mM to about 300 mM. In addition, any amount of NaCl intermediate to the recited values are intended to be included.

In one aspect, the invention provides a liquid pharmaceutical composition comprising a human anti-TNF-alpha antibody or antigen binding fragment thereof, (e.g., adalimumab), a polyol, without the addition of NaCl, in amounts sufficient to formulate an antibody for therapeutic use.

The present invention also provides liquid formulations comprising a human anti-TNF-alpha antibody or antigen binding fragment thereof, at a pH of about 5.0 to 6.4, and a turbidity of less than about 60 NTU after a standard 24 hour stir-stress assay, without the addition of NaCl (e.g., about 20, 21, 22, 23, 24, 25, 26, 27, 28, 29, 30, 31, 32, 33, 34, 35, 36, 37. 38, 39, 40, 41, 42, 43, 44, 45, 46, 47, 48, 49, 50, 51, 52, 53, 54, 55, 56, 57, 58, 59, 60, 61, 62, or 63 NTU). In another aspect, the invention provides liquid formulations comprising a human anti-TNF-alpha antibody or antigen binding fragment thereof, at a pH of about 5.0 to 6.4, and a turbidity of less than about 100 NTU after a standard 48 hour stir-stress assay, without the addition of NaCl (e.g., about 35, 36, 37. 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 NTU). In yet another aspect, the invention provides liquid formulations comprising a human anti-TNF-alpha antibody or antigen binding fragment thereof, at a pH of about 5.0 to 6.4, and a turbidity of less than about 40 NTU after 3 months storage at 5° C., 25° C., or 40° C., without the addition of NaCl (e.g., about 20, 21, 22, 23, 24, 25, 26, 27, 28, 29, 30, 31, 32, 33, 34, 35, 36, 37. 38, 39, 40, 41, 42, 43, 44, 45, 46, 47, 48, 49, 50, 51, 52, 53, 54, 55, 56, 57, 58, 59, 60 NTU).

A feature of the formulation of the invention is the inclusion of a polyol, e.g, a sugar alcohol, at a concentration of greater than 20 mg/mL. In one embodiment, the polyol is either sorbitol or mannitol. It should be noted that the addition of sorbitol or mannitol to protein solutions is not always associated with a gain in protein stability. For instance, sorbitol offered no advantage against precipitation of porcine growth hormone when evaluated during thermal or interfacial stress conditions—in contrast to Tween 20 and hydroxypropyl-β-cyclodextrin, respectively (Charman et al. (1993) Pharm Res.10(7):954-62).

In one embodiment a suitable polyol for use in the formulations of the invention is a sugar alcohol, e.g., mannitol or sorbitol. The liquid formulations of the invention comprising a polyol typically comprise more than about 20 mg of the polyol. In one embodiment, the formulations comprise more than about 30 mg/mL of the polyol. In another embodiment, the formulations comprise more than about 40 mg/mL of the polyol. In another embodiment, he formulations comprise about 40-45 mg/mL of the polyol, e.g., about 35, 36, 37, 38, 39, 40, 41, 42, 43, 44, 45, 46, 47, 48, 49, 50, 51, 52, 53, 54, or 55 mg/mL. In addition, ranges of values using a combination of any of the above recited values as upper and/or lower limits are intended to be included.

In certain embodiments of the invention, a liquid formulation is prepared comprising the antibody in a pH-buffered solution. The buffer of this invention has a pH ranging from about 4 to about 8, preferably from about 4.5 to about 7.0, more preferably from about 4.5 to about 6.0, even more preferably from about 4.8 to about 5.5, and most preferably has a pH of about 5.0 to about 6.4. In one embodiment, the pH of the formulation of the invention is about 5.2. In another embodiment, the pH of the formulation of the invention is about 6.0. Ranges intermediate to the above recited pH's are also intended to be part of this invention (e.g., 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4). Ranges of values using a combination of any of the above recited values as upper and/or lower limits are intended to be included, e.g., 5.2-5.8. Examples of buffers that will control the pH within this range include phosphate, acetate (e.g. sodium acetate), succinate (such as sodium succinate), gluconate, glutamate, histidine, citrate and other organic acid buffers.

In a particular embodiment of the invention, the formulation comprises a buffer system which contains citrate and/or phosphate to maintain the pH in a range of about 5.0 to about 6.4. In one embodiment, the pH of the formulation is about 5.2. In another embodiment, the pH of the formulation is about 6.0.

In another preferred embodiment, the buffer system includes citric acid monohydrate, sodium citrate, disodium phosphate dihydrate, and/or sodium dihydrogen phosphate dihydrate. In a further preferred embodiment, the buffer system includes about 1.15-1.45 mg/ml of citric acid (e.g., about 1.15, 1.20, 1.25, 1.30, 1.35, 1.40, or 1.45), about 0.2-0.4 mg/mL of sodium citrate dehydrate (e.g., about 0.2, 0.25, 0.3, 0.35, or 0.4), about 1.35-1.75 mg/mL of disodium phosphate dehydrate (e.g., 1.35, 1.40, 1.45, 1.50, 1.55, 1.60, 1.65, 1.70, or 1.75), about 0.75-0.95 mg/mL of sodium dihydrogen phosphate dehydrate (e.g., about 0.75, 0.80, 0.85, 0.9, or 0.95).

Values and ranges intermediate to the above recited concentrations are also intended to be part of this invention. In addition, ranges of values using a combination of any of the above-recited values as upper and/or lower limits are intended to be included, e.g., 1.20-1.40 mg/mL.

In other embodiments, the buffer system includes 1.3-1.31 mg/mL of citric acid (e.g., about 1.305 mg/mL). In another embodiment, the buffer system includes about 0.27-0.33 mg/mL of sodium citrate dehydrate (e.g., about 0.305 mg/mL). In one embodiment, the buffer system includes about 1.5-1.56 mg/mL of disodium phosphate dehydrate (e.g., about 1.53 mg/mL). In another embodiment, the buffer system includes about 0.83-0.89 mg/mL of sodium dihydrogen phosphate dihydrate (e.g., about 0.86 mg/mL).

A detergent or surfactant may also be added to the antibody formulation of the invention. Exemplary detergents include nonionic detergents such as polysorbates (e.g. polysorbates 20, 80, etc.) or poloxamers (e.g. poloxamer 188). The amount of detergent added is such that it reduces aggregation of the formulated antibody and/or minimizes the formation of particulates in the formulation and/or reduces adsorption. In a preferred embodiment of the invention, the formulation includes a surfactant which is a polysorbate. In another preferred embodiment of the invention, the formulation contains the detergent polysorbate 80. In one preferred embodiment, the formulation contains between about 0.1 and about 2.0 mg/mL of polysorbate 80, e.g., about 1 mg/mL.

Values and ranges intermediate to the above recited concentrations are also intended to be part of this invention, e.g., 0.2, 0.3, 0.4, 0.5, 0.6, 0.7, 0.8, 0.9, 1, 1.1, 1.2, 1.3, 1.4, 1.5, 1.6, 1.7, 1.8, 1.9. In addition, ranges of values using a combination of any of the above-recited values as upper and/or lower limits are intended to be included, e.g., 0.3 to 1.1 mg/mL.

In one embodiment, the formulation of the invention consists essentially of a human TNF alpha antibody, or antigen binding portion thereof, at a concentration of at least about 100 mg/mL, a surfactant (e.g., polysorbate 80), a polyol (e.g., more than 20 mg/mL of sorbitol or mannitol), and a buffering system (e.g., citric acid monohydrate, sodium citrate, disodium phosphate dihydrate, and/or sodium dihydrogen phosphate dihydrate), and does not contain NaCl.

In one embodiment, the formulation contains the above-identified agents (i.e., an antibody at a concentration of at least about 100 mg/mL, a buffer system, a polyol, and a surfactant, without NaCl) and is essentially free of preservatives, such as benzyl alcohol, phenol, m-cresol, chlorobutanol and benzethonium Cl. In another embodiment, a preservative may be included in the formulation. One or more other pharmaceutically acceptable carriers, excipients or stabilizers such as those described in Remington's Pharmaceutical Sciences 16th edition, Osol, A. Ed. (1980) may be included in the formulation provided that they do not significantly adversely affect the desired characteristics of the formulation. Acceptable carriers, excipients or stabilizers are nontoxic to recipients at the dosages and concentrations employed and include; additional buffering agents; co-solvents; antioxidants including ascorbic acid and methionine; chelating agents such as EDTA; metal complexes (e.g. Zn-protein complexes); biodegradable polymers such as polyesters; and/or salt-forming counterions such as sodium.

The formulation herein may also be combined with one or more other therapeutic agents as necessary for the particular indication being treated, preferably those with complementary activities that do not adversely affect the antibody of the formulation. Such therapeutic agents are suitably present in combination in amounts that are effective for the purpose intended. Additional therapeutic agents which can be combined with the formulation of the invention are further described in U.S. Pat. Nos. 6,090,382 and 6,258,562, each of which is incorporated herein by reference.

The formulations to be used for in vivo administration must be sterile. This is readily accomplished by filtration through sterile filtration membranes prior to, or following, preparation of the formulation.

As described above, the liquid formulation of the invention has advantageous stability and storage properties. Stability of the liquid formulation is not dependent on the form of storage, and includes, but is not limited to, formulations which are frozen, lyophilized, spray-dried, or formulations which in which the active ingredient is suspended. Stability can be measured at a selected temperature for a selected time period. In one aspect of the invention, the protein in the liquid formulations is stable in a liquid form for at least about 3 months; at least about 4 months, at least about 5 months; at least about 6 months; at least about 12 months; at least about 18 months. Values and ranges intermediate to the above recited time periods are also intended to be part of this invention, e.g., about 3, 4, 5, 6, 7, 8, 9, 10, 11, 12, 13, 14, 15, 16, 17, 18, 19, 20, 21, 22, 23, or about 24 months. In addition, ranges of values using a combination of any of the above recited values as upper and/or lower limits are intended to be included. Preferably, the formulation is stable at room temperature (about 30° C.) or at 40° C. for at least about 1 month and/or stable at about 2-8° C. for at least about 1 year, or more preferably stable at about 2-8° C. for at least about 2 years. Furthermore, the formulation is preferably stable following freezing (to, e.g., −80° C.) and thawing of the formulation, hereinafter referred to as a “freeze/thaw cycle.”

Stability of a protein in a liquid formulation may also be defined as the percentage of monomer, aggregate, or fragment, or combinations thereof, of the protein in the formulation. A protein “retains its physical stability” in a formulation if it shows substantially no signs of aggregation, precipitation and/or denaturation upon visual examination of color and/or clarity, or as measured by UV light scattering or by size exclusion chromatography. In one aspect of the invention, a stable liquid formulation is a formulation having less than about 10%, and preferably less than about 5% of the protein being present as aggregate in the formulation.

In one embodiment, the physical stability of a liquid formulation is determined by determining turbidity of the formulation following a stir stress assay, e.g., 24 hour or 48-hour stir-stress assay. For example, a stir stress assay may be performed by placing a suitable volume of a liquid formulation in a beaker with a magnetic stirrer, e.g., (multipoint HP, 550 rpm), removing aliquots at any suitable time, e.g., at T0-T48 (hrs), and performing suitable assays as desired on the aliquots. Samples of a formulation under the same conditions but without stirring serve as control.

Turbidity measurements may be performed using a laboratory turbidity measurement system from Hach (Germany) and are reported as nephelometric units (NTU).

The liquid formulations of the invention also have advantageous tolerability properties. Tolerability is evaluated based on assessment of subject-perceived injection site pain using the Pain Visual Analog Scale (VAS).

A (VAS) is a measurement instrument that measures pain as it ranges across a continuum of values, e.g., from none to an extreme amount of pain. Operationally a VAS is a horizontal line, about 100 mm in length, anchored by numerical and/or word descriptors, e.g., 0 or 10, or ‘no pain’ or ‘excruciating pain’, optionally with additional word or numeric descriptors between the extremes, e.g., mild, moderate, and severe; or 1 through 9) (see, e.g., Lee J S, et al. (2000) Acad Emerg Med 7:550).

Additional indicators of tolerability that may be measured include, for example, the Draize Scale (hemorrhage, petechiae, erythema, edema, pruritus) and bruising.

### III. Antibodies for Use in the Formulations of the Invention

Antibodies that can be used in the formulations of the invention are antibodies directed against the antigen TNF-alpha, including human TNF-alpha (or hTNF-alpha).

In one embodiment, the invention features an isolated human antibody, or antigen-binding portion thereof, that binds to human TNF-alpha with high affinity and a low off rate, and also has a high neutralizing capacity. Preferably, the human antibodies used in the invention are recombinant, neutralizing human anti-hTNF-alpha antibodies. The most preferred recombinant, neutralizing antibody of the invention is referred to herein as D2E7, also referred to as HUMIRA™ or adalimumab (the amino acid sequence of the D2E7 VL region is shown in SEQ ID NO: 1; the amino acid sequence of the D2E7 VH region is shown in SEQ ID NO: 2). The properties of D2E7 (adalimumab/HUMIRA®) have been described in Salfeld et al., U.S. Pat. Nos. 6,090,382, 6,258,562, and 6,509,015, which are each incorporated by reference herein.

In one embodiment, the human TNF-alpha, or an antigen-binding portion thereof, dissociates from human TNF-alpha with a Kd of 1×10-8 M or less and a Koff rate constant of 1×10-3 s-1 or less, both determined by surface plasmon resonance, and neutralizes human TNF-alpha cytotoxicity in a standard in vitro L929 assay with an IC50 of 1×10-7 M or less. More preferably, the isolated human antibody, or antigen-binding portion thereof, dissociates from human TNF-alpha with a Koff of 5×10-4 s-1 or less, or even more preferably, with a Koff of 1×10-4 s-1 or less. More preferably, the isolated human antibody, or antigen-binding portion thereof, neutralizes human TNF-alpha cytotoxicity in a standard in vitro L929 assay with an IC50 of 1×10-8 M or less, even more preferably with an IC50 of 1×10-9 M or less and still more preferably with an IC50 of 1×10-10 M or less. In a preferred embodiment, the antibody is an isolated human recombinant antibody, or an antigen-binding portion thereof.

It is well known in the art that antibody heavy and light chain CDR3 domains play an important role in the binding specificity/affinity of an antibody for an antigen. Accordingly, in another aspect, the invention pertains to treating Crohn's disease by administering human antibodies that have slow dissociation kinetics for association with hTNF-alpha and that have light and heavy chain CDR3 domains that structurally are identical to or related to those of D2E7. Position 9 of the D2E7 VL CDR3 can be occupied by Ala or Thr without substantially affecting the Koff. Accordingly, a consensus motif for the D2E7 VL CDR3 comprises the amino acid sequence: Q-R-Y-N-R-A-P-Y-(T/A) (SEQ ID NO: 3). Additionally, position 12 of the D2E7 VH CDR3 can be occupied by Tyr or Asn, without substantially affecting the Koff. Accordingly, a consensus motif for the D2E7 VH CDR3 comprises the amino acid sequence: V-S-Y-L-S-T-A-S-S-L-D-(Y/N) (SEQ ID NO: 4). Moreover, as demonstrated in Example 2 of U.S. Pat. No. 6,090,382, the CDR3 domain of the D2E7 heavy and light chains is amenable to substitution with a single alanine residue (at position 1, 4, 5, 7 or 8 within the VL CDR3 or at position 2, 3, 4, 5, 6, 8, 9, 10 or 11 within the VH CDR3) without substantially affecting the Koff. Still further, the skilled artisan will appreciate that, given the amenability of the D2E7 VL and VH CDR3 domains to substitutions by alanine, substitution of other amino acids within the CDR3 domains may be possible while still retaining the low off rate constant of the antibody, in particular substitutions with conservative amino acids. Preferably, no more than one to five conservative amino acid substitutions are made within the D2E7 VL and/or VH CDR3 domains. More preferably, no more than one to three conservative amino acid substitutions are made within the D2E7 VL and/or VH CDR3 domains. Additionally, conservative amino acid substitutions should not be made at amino acid positions critical for binding to hTNF alpha. Positions 2 and 5 of the D2E7 VL CDR3 and positions 1 and 7 of the D2E7 VH CDR3 appear to be critical for interaction with hTNF alpha and thus, conservative amino acid substitutions preferably are not made at these positions (although an alanine substitution at position 5 of the D2E7 VL CDR3 is acceptable, as described above) (see U.S. Pat. No. 6,090,382).

Accordingly, in another embodiment, the antibody or antigen-binding portion thereof preferably contains the following characteristics:

a) dissociates from human TNFα with a Koff rate constant of 1×10-3 s-1 or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

More preferably, the antibody, or antigen-binding portion thereof, dissociates from human TNF-alpha with a Koff of 5×10-4 s-1 or less. Even more preferably, the antibody, or antigen-binding portion thereof, dissociates from human TNF-alpha with a Koff of 1×10-4 s-1 or less.

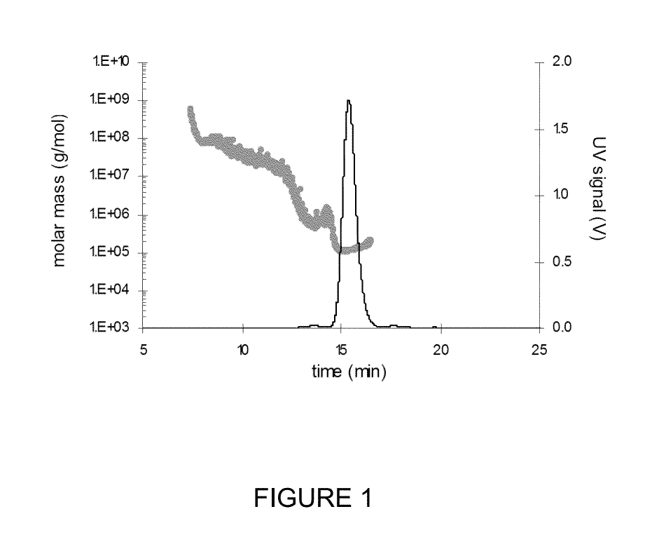

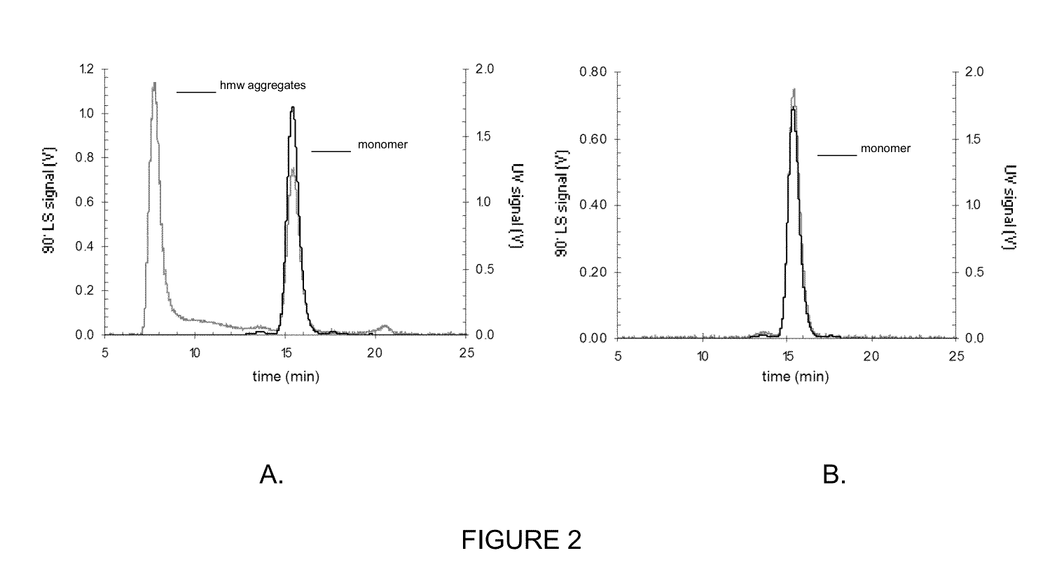

In yet another embodiment, the antibody or antigen-binding portion thereof preferably contains a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and with a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11. Preferably, the LCVR further has a CDR2 domain comprising the amino acid sequence of SEQ ID NO: 5 (i.e., the D2E7 VL CDR2) and the HCVR further has a CDR2 domain comprising the amino acid sequence of SEQ ID NO: 6 (i.e., the D2E7 VH CDR2). Even more preferably, the LCVR further has CDR1 domain comprising the amino acid sequence of SEQ ID NO: 7 (i.e., the D2E7 VL CDR1) and the HCVR has a CDR1 domain comprising the amino acid sequence of SEQ ID NO: 8 (i.e., the D2E7 VH CDR1). The framework regions for VL preferably are from the VκI human germline family, more preferably from the A20 human germline Vk gene and most preferably from the D2E7 VL framework sequences shown in FIGS. 1A and 1B of U.S. Pat. No. 6,090,382. The framework regions for VH preferably are from the VH3 human germline family, more preferably from the DP-31 human germline VH gene and most preferably from the D2E7 VH framework sequences shown in FIGS. 2A and 2B of U.S. Pat. No. 6,090,382.

Accordingly, in another embodiment, the antibody or antigen-binding portion thereof preferably contains a light chain variable region (LCVR) comprising the amino acid sequence of SEQ ID NO: 1 (i.e., the D2E7 VL) and a heavy chain variable region (HCVR) comprising the amino acid sequence of SEQ ID NO: 2 (i.e., the D2E7 VH). In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

In still other embodiments, the invention includes uses of an isolated human antibody, or an antigen-binding portion thereof, containing D2E7-related VL and VH CDR3 domains. For example, antibodies, or antigen-binding portions thereof, with a light chain variable region (LCVR) having a CDR3 domain comprising an amino acid sequence selected from the group consisting of SEQ ID NO: 3, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26 or with a heavy chain variable region (HCVR) having a CDR3 domain comprising an amino acid sequence selected from the group consisting of SEQ ID NO: 4, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35.

An antibody, or antibody portion, used in the methods and compositions of the invention, can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express adalimumab (D2E7) or an adalimumab (D2E7)-related antibody, DNA fragments encoding the light and heavy chain variable regions are first obtained. These DNAs can be obtained by amplification and modification of germline light and heavy chain variable sequences using the polymerase chain reaction (PCR). Germline DNA sequences for human heavy and light chain variable region genes are known in the art (see e.g., the “Vbase” human germline sequence database; see also Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline VH Sequences Reveals about Fifty Groups of VH Segments with Different Hypervariable Loops” J. Mol. Biol. 227:776-798; and Cox, J. P. L. et al. (1994) “A Directory of Human Germ-line V78 Segments Reveals a Strong Bias in their Usage” Eur. J. Immunol. 24:827-836; the contents of each of which are expressly incorporated herein by reference). To obtain a DNA fragment encoding the heavy chain variable region of D2E7, or a D2E7-related antibody, a member of the VH3 family of human germline VH genes is amplified by standard PCR. Most preferably, the DP-31 VH germline sequence is amplified. To obtain a DNA fragment encoding the light chain variable region of D2E7, or a D2E7-related antibody, a member of the VκI family of human germline VL genes is amplified by standard PCR. Most preferably, the A20 VL germline sequence is amplified. PCR primers suitable for use in amplifying the DP-31 germline VH and A20 germline VL sequences can be designed based on the nucleotide sequences disclosed in the references cited supra, using standard methods.

Once the germline VH and VL fragments are obtained, these sequences can be mutated to encode the D2E7 or D2E7-related amino acid sequences disclosed herein. The amino acid sequences encoded by the germline VH and VL DNA sequences are first compared to the D2E7 or D2E7-related VH and VL amino acid sequences to identify amino acid residues in the D2E7 or D2E7-related sequence that differ from germline. Then, the appropriate nucleotides of the germline DNA sequences are mutated such that the mutated germline sequence encodes the D2E7 or D2E7-related amino acid sequence, using the genetic code to determine which nucleotide changes should be made. Mutagenesis of the germline sequences is carried out by standard methods, such as PCR-mediated mutagenesis (in which the mutated nucleotides are incorporated into the PCR primers such that the PCR product contains the mutations) or site-directed mutagenesis.

Moreover, it should be noted that if the “germline” sequences obtained by PCR amplification encode amino acid differences in the framework regions from the true germline configuration (i.e., differences in the amplified sequence as compared to the true germline sequence, for example as a result of somatic mutation), it may be desirable to change these amino acid differences back to the true germline sequences (i.e., “backmutation” of framework residues to the germline configuration).

Once DNA fragments encoding D2E7 or D2E7-related VH and VL segments are obtained (e.g., by amplification and mutagenesis of germline VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, or antibody portions used in the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the D2E7 or D2E7-related light or heavy chain sequences, the expression vector may already carry antibody constant region sequences. For example, one approach to converting the D2E7 or D2E7-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors used in the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It is understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to hTNF alpha. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than hTNF alpha by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

In view of the foregoing, nucleic acid, vector and host cell compositions that can be used for recombinant expression of the antibodies and antibody portions used in the invention include nucleic acids, and vectors comprising said nucleic acids, comprising the human TNF alpha antibody adalimumab (D2E7). The nucleotide sequence encoding the D2E7 light chain variable region is shown in SEQ ID NO: 36. The CDR1 domain of the LCVR encompasses nucleotides 70-102, the CDR2 domain encompasses nucleotides 148-168 and the CDR3 domain encompasses nucleotides 265-291. The nucleotide sequence encoding the D2E7 heavy chain variable region is shown in SEQ ID NO: 37. The CDR1 domain of the HCVR encompasses nucleotides 91-105, the CDR2 domain encompasses nucleotides 148-198 and the CDR3 domain encompasses nucleotides 295-330. It will be appreciated by the skilled artisan that nucleotide sequences encoding D2E7-related antibodies, or portions thereof (e.g., a CDR domain, such as a CDR3 domain), can be derived from the nucleotide sequences encoding the D2E7 LCVR and HCVR using the genetic code and standard molecular biology techniques.

In one embodiment, the liquid pharmaceutical formulation comprises a human TNF alpha antibody, or antigen-binding portion thereof, that is a bioequivalent or biosimilar to the antibody adalimumab. In one embodiment, a biosimilar antibody is an antibody which shows no clinically meaningful difference when compared to a reference antibody, e.g., adalimumab. A biosimilar antibody has equivalent safety, purity, and potency as a reference antibody, e.g., adalimumab.

### IV. Administration of the Formulation of the Invention

An advantage of the formulation of the invention is that is may be used to deliver a high concentration of a human anti-TNF alpha antibody, or antigen-binding portion, (e.g., adalimumab) to a subject subcutaneously. Thus, in one embodiment, the formulation of the invention are delivered to a subject subcutaneously. In one embodiment, the subject administers the formulation to himself/herself.

In one embodiment, an effective amount of the formulation is administered. The language “effective amount” of the formulation is that amount necessary or sufficient to inhibit TNF-alpha activity, e.g., prevent the various morphological and somatic symptoms of a detrimental TNF-alpha activity-associated state. In another embodiment, the effective amount of the formulation is the amount necessary to achieve the desired result. An example of an effective amount of the formulation is an amount sufficient to inhibit detrimental TNF-alpha activity or treat a disorder in which TNF alpha activity is detrimental.

As used herein, the term “a disorder in which TNF-alpha activity is detrimental” is intended to include diseases and other disorders in which the presence of TNF-alpha. in a subject suffering from the disorder has been shown to be or is suspected of being either responsible for the pathophysiology of the disorder or a factor that contributes to a worsening of the disorder. Accordingly, a disorder in which TNF-alpha. activity is detrimental is a disorder in which inhibition of TNF-alpha. activity is expected to alleviate the symptoms and/or progression of the disorder. Such disorders may be evidenced, for example, by an increase in the concentration of TNF-alpha. in a biological fluid of a subject suffering from the disorder (e.g., an increase in the concentration of TNF-alpha. in serum, plasma, synovial fluid, etc. of the subject), which can be detected, for example, using an anti-TNF-alpha. antibody.

As described in the appended Examples below, one advantage of the formulations of the invention is the ability to prepare formulations comprising high concentrations of antibody without increasing the viscosity of the formualtion. Therefore, as also described below, the new formulations permit administration of high amounts (e.g., effective amounts) of antibody in smaller volumes as compared to prior commercial formulations, thereby decreasing pain.

In one embodiment, the effective amount of antibody may be determined according to a strictly weight based dosing scheme (e.g., mg/kg) or may be a total body dose (also referred to as a fixed dose) which is independent of weight. In one example, an effective amount of the formulation is 0.8 mL of the formulation containing a total body dose of about 80 mg of antibody (i.e., 0.8 mL of a 100 mg/mL antibody formulation of the invention). In another example, an effective amount of the formulation is 0.4 mL of the formulation of the invention containing a total body dose of about 40 mg of antibody (i.e., 0.4 mL of a 100 mg/mL antibody formulation of the invention). In yet another example, an effective amount of the formulation is twice 0.8 mL of the formulation containing a total body dose of about 160 mg of antibody (i.e., two units containing 0.8 mL each of a 100 mg/mL antibody formulation of the invention). In a further example, an effective amount of the formulation is 0.2 mL of the formulation of the invention containing a total body dose of about 20 mg of antibody (i.e., 0.2 mL of a 100 mg/mL antibody formulation of the invention). Alternatively, an effective amount may be determined according to a weight-based fixed dosing regimen (see, e.g., WO 2008/154543, incorporated by reference herein).

The invention provides a stable, high concentration formulation with an extended shelf life, which, in one embodiment, is used to inhibit TNF-alpha activity in a subject suffering from a disorder in which TNF-alpha activity is detrimental, comprising administering to the subject a formulation of the invention such that TNF-alpha activity in the subject is inhibited. Preferably, the TNF-alpha is human TNF-alpha and the subject is a human subject. Alternatively, the subject can be a mammal expressing a TNF-alpha with which an antibody of the invention cross-reacts. Still further the subject can be a mammal into which has been introduced hTNF-alpha (e.g., by administration of hTNF-alpha or by expression of an hTNF-alpha transgene).

A formulation of the invention can be administered to a human subject for therapeutic purposes (discussed further below). In one embodiment of the invention, the liquid pharmaceutical formulation is easily administratable, which includes, for example, a formulation which is self-administered by the patient. In a preferred embodiment, the formulation of the invention is administered through subcutaneous injection, preferably single use. Moreover, a formulation of the invention can be administered to a non-human mammal expressing a TNF-alpha with which the antibody cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration).

In one embodiment, the liquid pharmaceutical formulation of the invention may be administered to a subject via a prefilled syringe, an autoinjector pen, or a needle-free administration device. Thus, the invention also features an autoinjector pen, a prefilled syringe, or a needle-free administration device comprising the liquid pharmaceutical formulation of the invention. In one embodiment, the invention features a delivery device comprising a dose of the formulation comprising 100 mg/mL a human TNF alpha antibody, or antigen-binding portion thereof, e.g., an autoinjector pen or prefilled syringe comprises a dose of about 19 mg, 20, mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, etc. of the formulation.

Preferably, the formulation of the invention is used to treat disorders in which TNF alpha activity is detrimental. As used herein, the term “a disorder in which TNF-alpha activity is detrimental” is intended to include diseases and other disorders in which the presence of TNF-alpha in a subject suffering from the disorder has been shown to be or is suspected of being either responsible for the pathophysiology of the disorder or a factor that contributes to a worsening of the disorder. Accordingly, a disorder in which TNF-alpha activity is detrimental is a disorder in which inhibition of TNF-alpha activity is expected to alleviate the symptoms and/or progression of the disorder. Such disorders may be evidenced, for example, by an increase in the concentration of TNF-alpha in a biological fluid of a subject suffering from the disorder (e.g., an increase in the concentration of TNF-alpha in serum, plasma, synovial fluid, etc. of the subject), which can be detected, for example, using an anti-TNF-alpha antibody as described above.

There are numerous examples of disorders in which TNF-alpha activity is detrimental. Examples in which TNF-alpha activity is detrimental are also described in U.S. Pat. Nos. 6,015,557; 6,177,077; 6,379,666; 6,419,934; 6,419,944; 6,423,321; 6,428,787; and 6,537,549; and PCT Publication Nos. WO 00/50079 and WO 01/49321, the entire contents of all of which are incorporated herein by reference. The formulations of the invention may also be used to treat disorders in which TNF alpha activity is detrimental as described in U.S. Pat. Nos. 6,090,382, 6,258,562 and U.S. Patent Application Publication No. US20040126372, the entire contents of all of which are incorporated herein by reference.

The use of the formulations of the invention in the treatment of specific exemplary disorders is discussed further below:

A. Sepsis

Tumor necrosis factor has an established role in the pathophysiology of sepsis, with biological effects that include hypotension, myocardial suppression, vascular leakage syndrome, organ necrosis, stimulation of the release of toxic secondary mediators and activation of the clotting cascade (see e.g., Tracey, K. J. and Cerami, A. (1994) Annu. Rev. Med. 45:491-503; Russell, D. and Thompson, R. C. (1993) Curr. Opin. Biotech. 4:714-721). Accordingly, the formulation of the invention can be used to treat sepsis in any of its clinical settings, including septic shock, endotoxic shock, gram negative sepsis and toxic shock syndrome.

Furthermore, to treat sepsis, the formulation of the invention can be coadministered with one or more additional therapeutic agents that may further alleviate sepsis, such as an interleukin-1 inhibitor (such as those described in PCT Publication Nos. WO 92/16221 and WO 92/17583), the cytokine interleukin-6 (see e.g., PCT Publication No. WO 93/11793) or an antagonist of platelet activating factor (see e.g., European Patent Application Publication No. EP 374 510).

Additionally, in a preferred embodiment, the formulation of the invention is administered to a human subject within a subgroup of sepsis patients having a serum or plasma concentration of IL-6 above 500 pg/ml, and more preferably 1000 pg/ml, at the time of treatment (see PCT Publication No. WO 95/20978).

B. Autoimmune Diseases

Tumor necrosis factor has been implicated in playing a role in the pathophysiology of a variety of autoimmune diseases. For example, TNF-alpha has been implicated in activating tissue inflammation and causing joint destruction in rheumatoid arthritis (see e.g., Tracey and Cerami, supra; Arend, W. P. and Dayer, J-M. (1995) Arth. Rheum. 38:151-160; Fava, R. A., et al. (1993) Clin. Exp. Immunol. 94:261-266). TNF-alpha also has been implicated in promoting the death of islet cells and in mediating insulin resistance in diabetes (see e.g., Tracey and Cerami, supra; PCT Publication No. WO 94/08609). TNF-alpha also has been implicated in mediating cytotoxicity to oligodendrocytes and induction of inflammatory plaques in multiple sclerosis (see e.g., Tracey and Cerami, supra). Also included in autoimmune diseases that may be treated using the formulation of the invention is juvenile idiopathic arthritis (JIA) (also referred to as juvenile rheumatoid arthritis) (see Grom et al. (1996) Arthritis Rheum. 39:1703; Mangge et al. (1995) Arthritis Rheum. 8:211).

The formulation of the invention can be used to treat autoimmune diseases, in particular those associated with inflammation, including rheumatoid arthritis, rheumatoid spondylitis (also referred to as ankylosing spondylitis), osteoarthritis and gouty arthritis, allergy, multiple sclerosis, autoimmune diabetes, autoimmune uveitis, juvenile idiopathic arthritis (also referred to as juvenile rheumatoid arthritis), and nephrotic syndrome.

C. Infectious Diseases

Tumor necrosis factor has been implicated in mediating biological effects observed in a variety of infectious diseases. For example, TNF-alpha has been implicated in mediating brain inflammation and capillary thrombosis and infarction in malaria (see e.g., Tracey and Cerami, supra). TNF-alpha also has been implicated in mediating brain inflammation, inducing breakdown of the blood-brain barrier, triggering septic shock syndrome and activating venous infarction in meningitis (see e.g., Tracey and Cerami, supra). TNF-alpha also has been implicated in inducing cachexia, stimulating viral proliferation and mediating central nervous system injury in acquired immune deficiency syndrome (AIDS) (see e.g., Tracey and Cerami, supra). Accordingly, the antibodies, and antibody portions, of the invention, can be used in the treatment of infectious diseases, including bacterial meningitis (see e.g., European Patent Application Publication No. EP 585 705), cerebral malaria, AIDS and AIDS-related complex (ARC) (see e.g., European Patent Application Publication No. EP 230 574), as well as cytomegalovirus infection secondary to transplantation (see e.g., Fietze, E., et al. (1994) Transplantation 58:675-680). The formulation of the invention, also can be used to alleviate symptoms associated with infectious diseases, including fever and myalgias due to infection (such as influenza) and cachexia secondary to infection (e.g., secondary to AIDS or ARC).

D. Transplantation

Tumor necrosis factor has been implicated as a key mediator of allograft rejection and graft versus host disease (GVHD) and in mediating an adverse reaction that has been observed when the rat antibody OKT3, directed against the T cell receptor CD3 complex, is used to inhibit rejection of renal transplants (see e.g., Tracey and Cerami, supra; Eason, J. D., et al. (1995) Transplantation 59:300-305; Suthanthiran, M. and Strom, T. B. (1994) New Engl. J. Med. 331:365-375). Accordingly, the formulations of the invention can be used to inhibit transplant rejection, including rejections of allografts and xenografts and to inhibit GVHD. Although the antibody or antibody portion may be used alone, it can be used in combination with one or more other agents that inhibit the immune response against the allograft or inhibit GVHD. For example, in one embodiment, the formulations of the invention are used in combination with OKT3 to inhibit OKT3-induced reactions. In another embodiment, the formulation of the invention is used in combination with one or more antibodies directed at other targets involved in regulating immune responses, such as the cell surface molecules CD25 (interleukin-2 receptor-.alpha.), CD11a (LFA-1), CD54 (ICAM-1), CD4, CD45, CD28/CTLA4, CD80 (B7-1) and/or CD86 (B7-2). In yet another embodiment, the formulation of the invention is used in combination with one or more general immunosuppressive agents, such as cyclosporin A or FK506.

E. Malignancy

Tumor necrosis factor has been implicated in inducing cachexia, stimulating tumor growth, enhancing metastatic potential and mediating cytotoxicity in malignancies (see e.g., Tracey and Cerami, supra). Accordingly, the formulations of the invention can be used in the treatment of malignancies, to inhibit tumor growth or metastasis and/or to alleviate cachexia secondary to malignancy.

F. Pulmonary Disorders

Tumor necrosis factor has been implicated in the pathophysiology of adult respiratory distress syndrome, including stimulating leukocyte-endothelial activation, directing cytotoxicity to pneumocytes and inducing vascular leakage syndrome (see e.g., Tracey and Cerami, supra). Accordingly, the formulations of the invention can be used to treat various pulmonary disorders, including adult respiratory distress syndrome (see e.g., PCT Publication No. WO 91/04054), shock lung, chronic pulmonary inflammatory disease, pulmonary sarcoidosis, pulmonary fibrosis and silicosis.

G. Intestinal Disorders

Tumor necrosis factor has been implicated in the pathophysiology of inflammatory bowel disorders (see e.g., Tracy, K. J., et al. (1986) Science 234:470-474; Sun, X-M., et al. (1988) J. Clin. Invest. 81:1328-1331; MacDonald, T. T., et al. (1990) Clin. Exp. Immunol. 81:301-305) Chimeric murine anti-hTNF-alpha antibodies have undergone clinical testing for treatment of Crohn's disease (van Dullemen, H. M., et al. (1995) Gastroenterology 109:129-135). The formulation of the invention, also can be used to treat intestinal disorders, such as idiopathic inflammatory bowel disease, which includes two syndromes, Crohn's disease and ulcerative colitis.

H. Cardiac Disorders

The formulation of the invention, also can be used to treat various cardiac disorders, including ischemia of the heart (see e.g., European Patent Application Publication No. EP 453 898) and heart insufficiency (weakness of the heart muscle)(see e.g., PCT Publication No. WO 94/20139).

I. Spondyloarthropathies

TNFα has been implicated in the pathophysiology of a wide variety of disorders, including inflammatory diseases such as spondyloarthopathies (see e.g., Moeller, A., et al. (1990) Cytokine 2:162-169; U.S. Pat. No. 5,231,024 to Moeller et al.; European Patent Publication No. 260 610 B1 by Moeller, A). An example of a spondyloarthropathy that may be treated by the formulation of the invention includes psoriatic arthritis. Tumor necrosis factor has been implicated in the pathophysiology of psoriatic arthritis (Partsch et al. (1998) Ann Rheum Dis. 57:691; Ritchlin et al. (1998) J Rheumatol. 25:1544).

J. Skin and Nail Disorders

In one embodiment, the formulation of the invention is used to treat skin and nail disorders. As used herein, the term “skin and nail disorder in which TNFα activity is detrimental” is intended to include skin and/or nail disorders and other disorders in which the presence of TNF alpha in a subject suffering from the disorder has been shown to be or is suspected of being either responsible for the pathophysiology of the disorder or a factor that contributes to a worsening of the disorder, e.g., psoriasis. An example of a skin disorder which may be treated using the formulation of the invention is psoriasis. In one embodiment, the formulation of the invention is used to treat plaque psoriasis. Tumor necrosis factor has been implicated in the pathophysiology of psoriasis (Takematsu et al. (1989) Arch Dermatol Res. 281:398; Victor and Gottlieb (2002) J Drugs Dermatol. 1(3):264).

In one embodiment, the formulation of the invention is used to treat rheumatoid arthritis, psoriatic arthritis, or ankylosing spondylitis. The formulation of the invention comprising an isolated human TNF alpha antibody, or antigen-binding portion thereof, (e.g., adalimumab), may be administered to a human subject according to a dosing scheme and dose amount effective for treating rheumatoid arthritis, psoriatic arthritis, or ankylosing spondylitis. In one embodiment, a dose of about 40 mg of a human TNF alpha antibody, or antigen-binding portion thereof, (e.g., adalimumab) (e.g., 0.4 mL of a 100 mg/mL formulation of the invention) in the formulation of the invention is administered to a human subject every other week for the treatment of rheumatoid arthritis, psoriatic arthritis, or ankylosing spondylitis. In one embodiment, the formulation is administered subcutaneously, every other week (also referred to as biweekly, see methods of administration described in US20030235585, incorporated by reference herein) for the treatment of rheumatoid arthritis, ankylosing spondylitis, or psoriatic arthritis.

In one embodiment, the formulation of the invention is used to treat Crohn's disease. The formulation of the invention comprising an isolated human TNF alpha antibody, or antigen-binding portion thereof, (e.g., adalimumab), may be administered to a human subject according to a dosing scheme and dose amount effective for treating Crohn's disease. In one embodiment, a dose of about 160 mg of a human TNF alpha antibody, or antigen-binding portion thereof, (e.g., adalimumab) (e.g., 1.6 mL of a 100 mg/mL formulation of the invention) in the formulation of the invention is administered to a human subject initially at about day 1, followed by a subsequent dose of 80 mg of the antibody (e.g., 0.8 mL of a 100 mg/mL formulation of the invention) two weeks later, followed by administration of about 40 mg (e.g., 0.4 mL of a 100 mg/mL formulation of the invention) every other week for the treatment of Crohn's disease. In one embodiment, the formulation is administered subcutaneously, according to a multiple variable dose regimen comprising an induction dose(s) and maintenance dose(s) (see, for example, U.S. Patent Publication Nos. US20060009385 and US20090317399) for the treatment of Crohn's disease, each of which are incorporated by reference herein) for the treatment of Crohn's disease.

In one embodiment, the formulation of the invention is used to treat psoriasis. The formulation of the invention comprising an isolated human TNF alpha antibody, or antigen-binding portion thereof, (e.g., adalimumab), may be administered to a human subject according to a dosing scheme and dose amount effective for treating psoriasis. In one embodiment, an initial dose of about 80 mg of a human TNF alpha antibody, or antigen-binding portion thereof, (e.g., adalimumab) (e.g., 0.8 mL of a 100 mg/mL formulation of the invention) in the formulation of the invention is administered to a human subject, followed by a subsequent dose of 40 mg of the antibody (e.g., 0.4 mL of a 100 mg/mL formulation of the invention) every other week starting one week after the initial dose. In one embodiment, the formulation is administered subcutaneously, according to a multiple variable dose regimen comprising an induction dose(s) and maintenance dose(s) (see, for example, US 20060009385 and WO 2007/120823, each of which are incorporated by reference herein) for the treatment of psoriasis.

In one embodiment, the formulation of the invention is used to treat juvenile idiopathic arthritis (JIA). The formulation of the invention comprising an isolated human TNF alpha antibody, or antigen-binding portion thereof, (e.g., adalimumab), may be administered to a human subject according to a dosing scheme and dose amount effective for treating JIA. In one embodiment, 20 mg of a human TNF alpha antibody, or antigen-binding portion thereof, in the formulation of the invention (e.g., 0.2 mL of a 100 mg/mL formulation of the invention) is administered to a subject weighing 15 kg (about 33 lbs) to less than 30 kg (66 lbs) every other week for the treatment of JIA. In another embodiment, 40 mg of a human TNF alpha antibody, or antigen-binding portion thereof, in the formulation of the invention (e.g., 0.4 mL of a 100 mg/mL formulation of the invention) is administered to a subject weighing more than or equal to 30 kg (66 lbs) every other week for the treatment of JIA. In one embodiment, the formulation is administered subcutaneously, according to a weight-based fixed dose (see, for example, U.S. Patent Publication No. 20090271164, incorporated by reference herein) for the treatment of JIA.

In one embodiment, an isolated human TNF alpha antibody, or antigen-binding portion thereof, (e.g., adalimumab), may be administered to a human subject for treatment of a disorder associated with detrimental TNFa activity according to a monthly dosing schedule, whereby the antibody is administered once every month or once every four weeks. As described above, examples of disorders that may be treated according to a monthly dosing schedule include, but are not limited to, rheumatoid arthritis, ankylosing spondylitis, JIA, psoriasis, Crohn's disease, or psoriatic arthritis. Thus, the formulation of the invention comprising an isolated human TNF alpha antibody, or antigen-binding portion thereof, (e.g., adalimumab), may be administered to a human subject for treatment of a disorder associated with detrimental TNFa activity according to a monthly dosing schedule. In one embodiment, 80 mg of a human TNF alpha antibody, or antigen-binding portion thereof, in the formulation of the invention (e.g., 0.8 mL of a 100 mg/mL formulation of the invention) is administered to a subject having a disorder associated with detrimental TNFa activity.

Dose amounts described herein may be delivered as a single dose (e.g., a single dose of 40 mg in 0.4 mL or 80 mg dose in 0.8 mL), or, alternatively may be delivered as multiple doses (e.g., four 40 mg doses or two 80 mg doses for delivery of a 160 mg dose).

The formulation of the invention comprising an isolated human TNF alpha antibody, or antigen-binding portion thereof, (e.g., adalimumab) may also be administered to a subject in combination with an additional therapeutic agent. In one embodiment, the formulation is administered to a human subject for treatment of rheumatoid arthritis in combination with methotrexate or other disease-modifying anti-rheumatic drugs (DMARDs). In another embodiment, the formulation is administered to a human subject for treatment of JIA in combination with methotrexate or other disease-modifying anti-rheumatic drugs (DMARDs). Additional combination therapies are described in U.S. Pat. Nos. 6,258,562 and 7,541,031; and U.S. Patent Publication No. US20040126372, the entire contents of all of which are incorporated by reference herein.

The formulation of the invention comprising a human TNF alpha antibody, or antigen-binding portion thereof, may also be used to treat a subject who has failed previous TNF inhibitor therapy, e.g., a subject who has lost response to or is intolerant to infliximab.

The invention is further illustrated in the following examples, which should not be construed as further limiting.

### EXAMPLES

### Example 1

### Improving Stability of Human Anti-TNF Alpha Antibody Liquid Pharmaceutical Formulation

This Example provides results of experiments aimed at improving the stability of the pharmaceutical formulation of the antibody adalimumab.

### Materials and Methods

Adalimumab (subclass G₁, about 47 kDa) was formulated in a modified pharmaceutical formulation in order to generate a liquid parenteral dosage form at 50 mg/mL final drug concentration. Previous formulation experiments had determined that a phosphate/citrate buffer system was superior to other buffer systems in terms of protein stabilization of adalimumab. Consequently, improved stability was addressed via addition of excipients for a liquid 50 mg/mL dosage. All excipients used were of highest purity (“pro analysis” grade) and purchased from Merck KGaA, Darmstadt, Germany. Mannitol was sourced from Mallinckrodt Baker B. V., Deventer, Holland.

Analysis of visible particulate matter was conducted according to the regulation of Ph. Eur. 2002 (§2.9.20 Contamination with particulate matter—visible particles). Subvisible particulate matter analysis was determined by light obscuration (SVSS-C⁴⁰, PAMAS GmbH, Rutesheim, Germany). A Superose TM6 10/30 column (Amersham Pharmacia Europe GmbH, Freiburg, Germany) was used for SE-HPLC analysis (assessment of protein monomer content), applying a 0.5 mL/min flow rate of a PBS buffer with pH 7.5, and connected to UV₂₈₀ spectrophotometry, refractive index detection and MALS for on-line detection. Analysis of each sample was performed at least in triplicate. Except stated otherwise, for all SE-HPLC data Sᵣₑᵢ was below 0.13 and for all light obscuration data below 2.3.

Individual protein formulations were prepared via dilution of adalimumab concentrates (˜70 mg/mL) with excipient stock solutions. The 70 mg/mL adalimumab stock solution was prepared using a composition of citrate and phosphate buffer components (i.e., citric acid*H₂O, sodium citrate dehydrate, Na₂HPO₄*2 H₂O, NaH₂PO₄*2 H₂O) as listed in Table 16.

Excipient stock solutions were generated by excipient dissolution in phosphate/citrate buffer medium using a composition of citrate and phosphate buffer components (i.e., citric acid*H2O, sodium citrate dehydrate, Na₂HPO₄*2 H₂O, NaH₂PO₄*2 H₂O) as listed in Table 16. Prior to sterile filtration (0.2 μm, Minisart®, Sartorius AG, Goettingen, Germany), pH adjustment was performed by adding of acid/base specimen of buffer components. All formulations were prepared at least in duplicate, and generated via final sterile filtration of solution batches into heat-sterilized (180° C., 25 min) 2R glass vials (Schott Glas, Mainz, Germany) under aseptic laminar air flow conditions. Teflon coated butyl-rubber closures were sterilized via moist heat (121° C.) according to Ph. Eur. prior to usage.

The various formulations were subjected to 3 month-short-time storage at three different temperatures (5° C., 25° C., 40° C.).

Adalimumab concentrates were provided by diafiltration of adalimumab bulk solution via Vivaflow 50 units (cut-off 50 kDa, Vivascience G, Hannover, Germany), using phosphate/citrate buffer medium for buffer exchange. Current processes for concentration and buffer exchange of biopharmaceutical solutions are based on IEX, SE-HPLC, ultra-/diafiltration and tangential flow filtration (Christy et al. (2002) Desalination, 144:133-136). Diafiltration was applied because purification, concentration and buffer exchange are possible within a single-unit operation with variable flow dynamics, thus minimizing protein stress (Table 1).

TABLE 1
Correlation Of Protein Loss And Number Of
Diafiltration Cycles.

| Number of | Protein Conc. |
|---|---|
| Diafiltration Cycles | (mg/mL) |
| 1 | 72.81 |
| 2 | 72.7 |
| 3 | 72.51 |
| 4 | 72.34 |
| 5 | 72.02 |
| 6 | 71.79 |
| 7 | 71.53 |
| 8 | 71.25 |
| 9 | 71 |
| 10 | 70.67 |

Each cycle performed accounted for a protein loss of ˜0.25% of total protein. Generally, protein loss did not exceed 7% in the course of concentrate production.

Within one diafiltration cycle, protein concentration was doubled and re-diluted to the original concentration, except for the terminal concentration step. Hence, undesirable dissolved substances not intended for presence can effectively be removed (e.g., a 1.00% concentration can be downsized to 0.00098% within ten diafiltration cycles). Subsequent to purification and concentration, the adalimumab concentrates were centrifugated (5° C., 3000 g, 20 minutes).

### Evaluation of pH Optimum

In order to evaluate the optimal solution pH (i.e., pH 5.2 or pH 6.0), two different adalimumab formulations were analyzed, varying solely in pH. Stability data of formulations containing 1 mg/mL Tween 80 are illustrated in Tables 2A and 2B.

TABLE 2A
Influence Of Formulation pH On Monomer Content
During 40° C. Storage.

| | Monomer Content (%) | Monomer Content (%) |
|---|---|---|
| Storage Time (w) | at pH 5.2 | at pH 6.0 |
| 0 | 98.9 | 98.86 |
| 1 | 98.59 | 98.19 |
| 4 | 97.54 | 97.01 |
| 12 | 95.53 | 95.53 |

TABLE 2B
Influence Of Formulation pH on Subvisible Particulate
Matter Formation During Storage.

| | Subvisible Particles > | Subvisible Particles > |
|---|---|---|
| | 1 μm/mL Content at | 1 μm/mL Content at |
| Storage Temp. (° C.) | pH 5.2 | pH 6.0 |
| 5 | 3564 | 179329 |
| 25 | 2547 | 50898 |
| 40 | 1532 | 36556 |

With respect to monomer content, no pH was found to be superior to another, as both formulations exhibited comparable monomer losses at 40° C. storage. Data of 25° C. storage conditions were similar to 40° C. data, whereas at 5° C. all protein solutions analyzed in the course of this study underwent no significant alterations in monomer content.

Differences were found in turbidity, however. A 6.0 solution pH resulted in the formation of subvisible particulate matter during 12 weeks of storage, regardless of the storage temperature. As the intensity of particulate matter formation is connected with lower temperatures, the particles' origin is not assumed to be proteineic. In that regard, if severe particulate matter formation were merely due to protein instability, this would be associated with exposure to elevated temperatures during storage tests (Constantino, et al. (1994b) J. Pharm. Sci. 83: 1662-1669).

With respect to 50 mg/mL adalimumab formulations containing 6.16 mg/mL NaCl instead of Tween 80, the addition of salt resulted in the formation of subvisible particles, as the number of particles greater than 1 μm was increased by a similar degree in both solutions (see Tables 3A and 3B). Furthermore, after 12 weeks, SE-HPLC data showed that the pH 6.0 solutions had a greater monomer content than solutions at pH 5.2, although the differences were minimal (˜0.3%) and not corroborated by 25° C. results.

TABLE 3A
Influence Of pH On Monomer Content During 40° C. Storage.

| | Monomer Content (%) | Monomer Content (%) |
|---|---|---|
| Storage Time (w) | at pH 5.2 | at pH 6.0 |
| 0 | 98.9 | 98.7 |
| 1 | 98.59 | 98.11 |
| 4 | 97.46 | 96.97 |
| 12 | 95.29 | 95.22 |

TABLE 3B
Influence Of pH On Subvisible Particulate Matter
Formation (B) During storage.

| | Subvisible Particles > | Subvisible Particles > |
|---|---|---|
| | 1 μm/mL Content at | 1 μm/mL Content at |
| Storage Temp. (° C.) | pH 5.2 | pH 6.0 |
| 5 | 127707 | 241222 |
| 25 | 17760 | 80404 |
| 40 | 91356 | 180084 |

Particle formation appeared to be facilitated by NaCl addition and pH 6.0 storage, and improved with Tween 80 addition and a solution pH of 5.2. Thus, Tween 80 was proposed as an ingredient that could alleviate particle contamination in solutions containing salts, such as NaCl (Tables 4A and 4B). Solutions were then examined that contained both 6.16 mg/mL NaCl and 1 mg/mL Tween 80.

TABLE 4A
Influence Of pH On Monomer Content During Storage.

| | Monomer Content (%) | Monomer Content (%) |
|---|---|---|
| Storage Time (w) | at pH 5.2 | at pH 6.0 |
| 0 | 98.9 | 98.7 |
| 1 | 98.59 | 98.11 |
| 4 | 97.46 | 96.97 |
| 12 | 95.29 | 95.22 |

TABLE 4B
Influence Of pH On Subvisible Particulate Matter Formation
During 40° C. Storage.

| | Subvisible Particles > | Subvisible Particles > |
|---|---|---|
| Storage | 1 μm/mL Content at | 1 μm/mL Content at |
| Temp. (° C.) | pH 5.2 | pH 6.0 |
| 5 | 152196 | 365213 |
| 25 | 61622 | 141182 |
| 40 | 111053 | 249876 |

As shown in Table 4B, for formulations comprising salt and surfactant, the addition of surfactant had no influence in terms of subvisible particle formation, as subvisible particles were apparent despite the addition of Tween 80. Interestingly, in all samples particle numbers were maximal at lowest storage temperature (5° C.), indicating the particle origin to be potentially due to inorganic material. Moreover, visible inspection of solutions containing salt revealed a slight turbidity after 4 week storage, regardless of the storage temperature. Precipitation of visible inorganic components can be the result of storage at cold temperatures, even if the storage is temporary, e.g., sodium phosphate buffers may yield the relatively insoluble Na₂HPO₄*12H₂O at 4° C. (Borchert et al. (1986) PDA J. Pharm. Sci. Technol., 40:212-241). However, in terms of particulate matter being an evaluating criterion, a solution pH of 5.2 had advantages over pH 6.0 for the examined solutions.

With respect to monomer content, however, both solution pH values rendered identical monomer contents during storage and in case of NaCl-containing formulations (without Tween 80) a pH of 6.0 appeared to reveal even slightly higher stability. Despite this similar monomer profile, it is commonly accepted that at pH values towards neutral or even basic conditions proteins are prone to a broader variety of potential degradation mechanisms (Wang (1999) Int. J. Pharm., 185:129-188) e.g., carbonyl-amine reactions of un-ionized protein amides, (base-catalyzed) β-eliminations and deamidations are facilitated by higher pH values as well as various oxidation reactions (Akers and DeFelippis, Peptides and proteins as parenteral solutions, in Pharmaceutical formulation development of peptides and proteins, ed. by Frokjaer, S; Hovgaard, L. (2000) 145-177). Hence, in summary, a solution pH of 5.2 was considered superior to a 6.0 value in terms of adalimumab 50 mg/mL long-time stability.

### Stabilization by Excipients: Surfactants

In order to determine the stabilizing potential of surfactants on 50 mg/mL adalimumab formulation, various amounts of Tween 80 (0.0%, 0.03%, 0.1%) were added to a protein solution containing 6.16 mg/mL NaCl. Generally, Tween 80 is assumed to stabilize proteins e.g., by binding through hydrophobic surface interaction. As a protein's surface characteristics are influenced by the presence of salts, the effect of the absence of NaCl additionally was surveyed (described as 0.1% Tween 80 solution without NaCl in Table 5) (see also Kheirolomoom et al. (1998) Biochem. Eng. J., 2:81-88).

TABLE 5
Influence Of Tween 80 On Protein Formulations Containing 6.16 mg/mL NaCl
(Storage Temperature 40° C.).

| | | | | Monomer Content |
|---|---|---|---|---|
| | Monomer Content | Monomer Content | Monomer Content | (%) 0.1% Tween, no |
| Storage Time (w) | (%) 0% Tween | (%) 0.03% Tween | (%) 0.1% Tween | NaCl |
| 0 | 98.86 | 98.91 | 98.9 | 98.9 |
| 1 | 98.55 | 98.58 | 98.59 | 98.59 |
| 4 | 97.39 | 97.49 | 97.46 | 97.54 |
| 12 | 95.18 | 92.55 | 95.29 | 95.53 |

The results from varying amounts of Tween 80 with and without NaCal are presented in Table. 5. As shown, Tween 80 was unable to provide stability to the formulation with or without NaCl. With respect to 0.03% Tween 80/NaCl, the combination resulted in decreasing the monomer content after 12 weeks of storage at 40° C. This result contradicted the majority of articles addressing this topic, as generally the stabilizing impact of Tween 80 is related to increasing concentrations of surfactant (valid in the range from 0.001 to 1%) (see Arakawa et al. (2001) Adv. Drug Deliv. Rev., 46:307-326).

In addition to monomer concentration at varying Tween 80 percentages with and without NaCl, subvisible particle formation was also examined at varying temperatures (see Table 6). At all storage temperatures, the addition of Tween 80 led to a substantial increase in subvisible particle numbers, especially at concentrations of 0.03% which confirmed the findings of SE-HPLC analysis. Interestingly, the absence of NaCl proved to notably decrease the formation of subvisible particles, regardless of the storage temperature.

TABLE 6
Influence of Tween 80 On Subvisible Particulate Matter Formation During
40° C. Storage Of Solutions Containing 6.16 mg/mL NaCl.

| | Subvisible | Subvisible | Subvisible | Subvisible Particles > |
|---|---|---|---|---|
| | Particles > 1 μm/ | Particles > 1 μm/ | Particles > 1 μm/ | 1 μm/mL Content |
| | mL Content | mL Content | mL Content | (%) 0.1% Tween, |
| Storage Temp. (° C.) | (%) 0% Tween | (%) 0.03% Tween | (%) 0.1% Tween | no NaCl |
| 5 | 127707 | 203884 | 152196 | 3564 |
| 25 | 17760 | 529244 | 61622 | 2547 |
| 40 | 91356 | 360929 | 111053 | 1533 |

The various concentrations of Tween 80 were also examined with respect to particulate formation following freeze/thaw cycles. In contrast to the minor stabilizing impact on liquid solutions during storage, Tween 80 proved to confer notable stability towards adalimumab during freeze-thaw cycles (Table 7).

TABLE 7
Stressing Protein Solutions With Varying Contents of Tween 80 By
Means of Freeze-Thaw Cycles.

| | Subvisible | Subvisible | Subvisible |
|---|---|---|---|
| Number of | Particles > 1 | Particles > 1 | Particles > 1 |
| Freeze/Thaw | μm/mL Content | μm/mL Content | μm/mL Content |
| Cycles | (%) 0% Tween | (%) 0.03% Tween | (%) 0.1% Tween |
| 0 | 5996 | 5391 | 5449 |
| 1 | 6178 | 6360 | 5049 |
| 2 | 13526 | 14520 | 6582 |
| 3 | 25509 | 26508 | 7850 |
| 4 | 38564 | 48392 | 8012 |
| 5 | 60507 | 69810 | 9533 |
| 6 | 69942 | 94742 | 12991 |
| 7 | 76209 | 99787 | 18111 |

The effect of Tween 80 was also determined by repeatedly subjecting the solutions to stress via freezing (−80° C., 12 hours) and thawing (5° C., 12 hours). The number of freeze-thaw (freeze/thaw) cycles applied was closely correlated to a gain in subvisible particulate matter. However, whereas the effect of 5 freeze/thaw cycles on solutions with 0 or 0.03% Tween 80 content resulted in a ˜10-fold increase in particle contamination (particles ≧1 μm), the situation virtually remained unchanged in 0.1% Tween 80 solutions. SE-HPLC analysis confirmed these results (Table 8).

TABLE 8
Loss Of Monomer In Adalimumab Solutions Varying In Tween 80
Content Independent On The Number Of Freeze-Thaw Cycles Exerted.

| Number of | | | |
|---|---|---|---|
| Freeze/ | Monomer Content | Monomer Content | Monomer Content |
| Thaw Cycles | (%) 0% Tween | (%) 0.03% Tween | (%) 0.1% Tween |
| 0 | 98.41 | 98.48 | 98.43 |
| 1 | 98.29 | 98.38 | 98.42 |
| 2 | 98.33 | 98.45 | 98.41 |
| 3 | 98.3 | 98.46 | 98.43 |
| 4 | 98.29 | 98.46 | 98.45 |
| 5 | 98.22 | 98.45 | 98.42 |
| 6 | 98.15 | 98.49 | 98.41 |
| 7 | 98.12 | 98.48 | 98.42 |

In close accordance to the results of numerous studies published on the effect of freeze/thaw cycles on other proteins, the stability of 50 mg/mL adalimumab decreased when exposed to repeated freeze/thaw stress when no surfactant was present. Conversely, the addition of surfactant shielded the protein against deleterious parameters associated with freezing/thawing, as the content of native monomer (verified using multi-angle light scattering (MALS)) remained unchanged.

In summary, the addition of 0.1% Tween 80 to adalimumab 50 mg/mL solutions was preferred. Though 0.1% Tween improved the protein stability in stored liquids only marginally, the stabilizing effects during processes such as freezing and thawing were substantial. Nevertheless, addition of Tween 80 may emerge as a great benefit, as freezing is a common unit operation in the production, storage and transport of protein pharmaceuticals (Cao et al. (2003) Biotechnol. Bioeng., 82:684-690). Additionally, the use of 0.1% Tween 80 in pharmaceuticals is well-accepted, demonstrated by the FDA approval of Orthoclone™ (murine IgG2a) as early as 1986.

Besides Tween 80, the nonionic surfactant Solutol® HS15 was investigated for its potential to stabilize adalimumab. The protecting features of Solutol® in concentrations of 0.03 and 0.1% were shown recently in terms of aviscumin parenterals (Steckel et al. (2003) Int. J. Pharm., 257:181-194). Hence, the influence of Solutol® on adalimumab solutions in terms of the formation of particulate matter contamination were compared to protein solutions containing 0.1% Tween 80 (Table 9).

TABLE 9
Influence Of Adalimumab Solutions Containing Various Solutol ®
Concentrations On Formation Of Particulate Matter After 12 Weeks Storage As
Compared To Adalimumab Solutions Containing 0.1% Tween 80.

| | Subvisible | Subvisible | Subvisible | |
|---|---|---|---|---|
| | Particles > 1 | Particles > 1 | Particles > 1 | Subvisible |
| | μm/mL Content | μm/mL Content | μm/mL Content | Particles > 1 |
| | (%) Solutol 0.3 | (%) Solutol 1 | (%) Solutol 10 | μm/mL Content |
| Storage Temp. (° C.) | mg/mL | mg/mL | mg/mL | (%) 0.1% Tween |
| 5 | 52760 | 57049 | 196929 | 152000 |
| 25 | 2978 | 1840 | 6827 | 61000 |
| 40 | 3884 | 1258 | 91333 | 111000 |

In contrast to solutions with 0.03% and 0.1% Solutol®, adalimumab solutions with 1% Solutol® and 0.1% Tween 80, respectively, exhibited a notable increase of particulate matter during storage. This positive influence of low Solutol® concentrations was not reflected in data of SE-HPLC analysis. After 12 week storage (40° C.), all solutions containing Solutol® revealed a loss in monomer content of ˜0.5% in comparison to the reference (0.1% Tween 80). (FIG. 1).

This experiment also illustrated the great advantages offered by MALS in the early-stage detection of high molecular weight (hmw) protein aggregates (FIGS. 2A and 2B). Due to its high sensitivity on large analytes, minimal concentrations are sufficient to detect aggregates by MALS, e.g., the formation of hmw aggregates after 1 week storage (40° C.) could be verified by MALS—but was virtually undetectable by UV₂₈₀-detection.

As a consequence, Solutol was removed from the list of potential stabilizers, as the formation of hmw aggregates already in early stages of accelerated shelf life studies is generally not acceptable. Even minimal amounts of protein (<0.1%) are known to account for precipitation (Hoffman, Analytical methods and stability testing of biopharmaceuticals, in Protein formulation and delivery, ed. by McNally, E. J., 3 (2000) 71-110). The findings above confirm previous studies that showed that higher concentrations (>1%) of Solutol® HS15 destabilized solutions of serpine-related protease inhibitor and availed visible particulate matter phenomena (see, e.g., WO 2006037606).

### Stabilization by Excipients: Polyols

Many sugars (e.g., sucrose, glucose, raffinose, trehalose) and polyols (e.g., glycerol, sorbitol, mannitol) are subsumed under the category of protein stabilizing co-solvents. It is widely believed that these substances act primarily through a steric exclusion mechanism. For example, polyols such as sorbitol are often used to stabilize parenterals, for instance in a number of lyophilized vaccine pharmaceuticals such as Mumpsvax™, Meruvax™ II and Attenuvax™ or intravenous administrable solutions such as Cardene™.

In contrast to other excipients such as surfactants, sugars and polyols must be added in higher concentrations (>0.5 M) in order to deploy their complete stabilizing potential. As a consequence, sorbitol at concentrations of 50 and 100 mg/mL was added to adalimumab solutions, and subjected to 12 weeks of storage (Table 10).

TABLE 10
Influence Of Sorbitol On Particulate Matter Formation In Adalimumab
Solutions During Storage For 12 Weeks.

| | Subvisible | Subvisible | |
|---|---|---|---|
| | Particles > 1 | Particles > 1 | Subvisible |
| | μm/mL Content | μm/mL Content | Particles > 1 |
| | (%) Sorbitol 50 | (%) Sorbitol 100 | μm/mL Content |
| Storage Temp. (° C.) | mg/mL | mg/mL | (%) No Sorbitol |
| 5 | 1000 | 3040 | 152196 |
| 25 | 778 | 2800 | 61622 |
| 40 | 2636 | 460 | 111053 |

Sorbitol decreased the tendency for particle formation during storage, compared to solutions where no sorbitol was present. The amount of added sorbitol did virtually not result in any differences. Regarding monomer content, the stabilizing effect of sorbitol was found to be closely concentration-dependent. The presence of NaCl detracts from protein stability (Table 11).

TABLE 11
Adalimumab Stability Is Dependent On Sorbitol Concentration, Reflected
By Content Of Protein Monomer (Numbers Indicate Concentrations In mg/mL; Storage
At 40° C.).

| | | | | Monomer Content |
|---|---|---|---|---|
| | | Monomer Content | Monomer Content | (%) Sorbitol 50 |
| | Monomer Content | (%) Sorbitol 100 | (%) Sorbitol 50 | mg/mL/4 mg/mL |
| Storage time (w) | (%) No Sorbitol | mg/mL | mg/mL | NaCl |
| 0 | 99.66 | 99.65 | 99.65 | 99.66 |
| 1 | 99.09 | 99.2 | 99.19 | 99.13 |
| 4 | 97.93 | 98.41 | 98.38 | 98.1 |
| 8 | 96.52 | 97.54 | 97.48 | 96.98 |
| 12 | 95.32 | 96.8 | 96.49 | 96.13 |

According to Table 11, the addition of 100 mg/mL sorbitol increased the content of monomer content by ˜1.5% during 12 week storage at 40° C. Reducing the amount of excipient lead to a reduction of adalimumab stability. These findings corroborate recent investigations on the stability of horse immunoglobulins, where 180 mg/mL sorbitol was shown to be superior to the addition of 90 mg/mL in terms of protein stabilization against heat stress (Rodrigues-Silva et al., 1999 Toxicon 37(1), 33-45). The concentration dependence of the stabilization of sugars and sugar-derived polyols has been reported (Chan et al. (1996) Pharm. Res., 13:756-761; Fatouros et al. (1997b) Pharm. Res., 14:1679-1684). Interestingly, the addition of 4 mg/mL salt detracted notably from the stabilizing potential of sorbitol (˜0.25% monomer), as shown in Table 11. On the other hand, the absence of NaCl in adalimumab solutions containing 0.1% Tween 80 led to only a minimal increase in monomer content during shelf life experiments (as shown in Table 11).

As shown in Table 12, the experiments were repeated with mannitol instead of sorbitol. The findings on sorbitol were substantiated by addition of mannitol to adalimumab solutions: (1) solutions enriched by 80 mg/mL mannitol exceeded mannitol-free solutions in protein monomer content by ˜1.5% after 12 weeks of storage (40° C.), (2) the stabilizing input of mannitol was oriented towards a concentration-dependent profile, and (3) NaCl reduced the decreasing monomer content of mannitol alone. Interestingly, these data were corroborated by identical experiments performed at 25° C.

TABLE 12
Adalimumab Stability Was Dependent On Mannitol Concentration,
Reflected By Content Of Protein Monomer (Numbers Indicate
Concentrations In mg/mL; Storage At 40° C.).

| | | | | Monomer |
|---|---|---|---|---|
| | Monomer | Monomer | Monomer | Content |
| | Content | Content | Content | (%) Mannitol |
| Storage | (%) No | (%) Mannitol | (%) Mannitol | 40 mg/mL/ |
| time (w) | Mannitol | 80 mg/mL | 40 mg/mL | 4 mg/mL NaCl |
| 0 | 99.66 | 99.67 | 99.66 | 99.69 |
| 1 | 99.09 | 99.2 | 99.18 | 99.14 |
| 4 | 97.93 | 98.36 | 98.31 | 98.1 |
| 8 | 96.52 | 97.46 | 97.48 | 97.05 |
| 12 | 95.32 | 96.81 | 96.37 | 96.26 |

In summary, adalimumab at a concentration of 50 mg/mL was stabilized by both sorbitol and mannitol. This stabilization was impeded by NaCl. The findings that NaCl does not impede adalimumab stability when added to protein solutions containing 0.1% Tween 80 was consistent with the conclusions above.

As shown in Table 13, the amount of native monomer in each adalimumab formulation was dependent on the addition of polyols and on the excipient composite. Commensurately, the amounts of aggregates and fragments varied. The aggregate share in the amount of monomer loss remained constant, regardless of the excipients added, if any. In other words, the ratio of adalimumab aggregates:fragments were in balance (i.e., ˜38% aggregates and ˜72% fragments), and this equilibrium was not influenced by the addition of polyols and salts. If sorbitol and mannitol were contributing to adalimumab stability solely via native state stabilization, this should be reflected in alterations of the aggregate share. Since this was not the case, there has to be a further mechanism of adalimumab stabilization by sorbitol/mannitol, resulting in an impediment of the fragmentation processes.

TABLE 13
Impact Of Excipient Addition On Adalimumab Stability After
12 Weeks Of Storage At 40° C. (Data Derived Via SE-HPLC)

| | | | | Aggregate |
|---|---|---|---|---|
| | | | | Share (%) |
| | | | | In The |
| | | | | AmountOf |
| | % | % | % | Monomer |
| Excipients | Monomer | Aggregate | Fragment | Loss |
| no excipient | 95.32 | 1.68 | 3.02 | 35.7 |
| sorbitol 50 mg/mL | 96.49 | 1.40 | 2.11 | 39.9 |
| sorbitol 50 mg/mL | 96.13 | 1.38 | 2.49 | 35.7 |
| NaCl 4 mg/mL | | | | |
| sorbitol 100 mg/mL | 96.80 | 1.21 | 1.99 | 37.8 |
| mannitol 40 mg/mL | 96.37 | 1.42 | 2.21 | 39.1 |
| mannitol 40 mg/mL | 96.46 | 1.40 | 2.34 | 37.4 |
| NaCl 4 mg/mL | | | | |
| mannitol 80 mg/mL | 96.81 | 1.28 | 1.91 | 39.9 |

In conclusion, adalimumab at a concentration of 50 mg/mL was effectively stabilized by adding mannitol or sorbitol to the formulation. Besides contributing to protein stability by native state protection, mannitol and sorbitol stabilized the protein via a further mechanism, thereby reducing fragmentation during long-term storage.

### Stabilization by Excipients: Salts

NaCl is the most-used salt in the formulation of protein parenterals. Nevertheless, the above results show that, at an adalimumab concentration of 50 mg/mL, NaCl impeded adalimumab stability in the presence of polyols, and did not increase protein stability as a sole excipient. When considering the potential stabilizing effect of salts, consideration of their behaviour in accordance with the Hoffmeister lyotropic series provided a rough rule of thumb. Thus, the use of anionic acetate instead of chloride as counterion in sodium salts was investigated.

As illustrated in Table 14, the individual solutions (i.e., 50 mg/mL sorbitol/4 mg/mL Na-acetate, 50 mg/mL sorbitol/4 mg/mL NaCal, and 50 mg/mL sorbitol, no salt) revealed different protein stability. The adalimumab solution containing NaCl was stacked against protein stability, since after only 4 weeks of storage (40° C.) a comparison of formulations containing either NaCl or sodium acetate showed that the monomer content in the sodium acetate enriched batch was ˜0.25% greater than that of the NaCl containing formulation, adding up to a >0.4% difference after 12 weeks. Consequently, sodium acetate contributed more to adalimumab stability than sodium chloride. Nevertheless, the addition of sodium acetate did not increase protein stabilization, since the salt-free formulation had identical monomer content.

TABLE 14
Stability Of Adalimumab In Solutions Containing Sorbitol
Is Dependent On Salt Addition (Numbers Indicate
Concentrations In mg/mL; Storage At 40° C.).

| | Monomer Content | Monomer Content | Monomer |
|---|---|---|---|
| | (%) 50 mg/mL | (%) 50 mg/mL | Content |
| Storage | Sorbitol/4 mg/mL | Sorbitol/4 mg/mL | (%) 50 mg/mL |
| time (w) | Na-acetate | NaCl | Sorbitol-No salt |
| 0 | 99.66 | 99.66 | 99.65 |
| 1 | 99.21 | 99.13 | 99.19 |
| 4 | 98.36 | 98.1 | 98.38 |
| 8 | 97.34 | 96.98 | 97.48 |
| 12 | 96.46 | 96.13 | 96.49 |

In comparison to both other formulations (formulations with 50 mg/mL Sorbitol and wither no salt of 4 mg/mL NaCl), acetate containing formulations exhibited a greater number of particles beyond 1 μm (˜180,000/mL versus <6,000/mL).

Buffer systems were also examined, whereby sodium and potassium buffer systems were compared with varying concentrations of sorbitol. As illustrated in Table 15, the stability of adalimumab dissolved in potassium phosphate buffer equaled that determined in sodium phosphate buffers. Data of storage tests performed at 25° C. substantiated these findings. Additionally, both buffer systems equaled in particulate matter contamination. Thus, potassium phosphate was considered to be preferred in liquid protein formulations.

TABLE 15
Adalimumab Stability In Phosphate Buffer Systems Using Sodium And
Potassium As Cationic Counterions (Buffer Concentration ~ 10 Mm, Numbers
Indicate Sorbitol Concentrations In mg/mL; Storage At 40° C.).

| | Monomer Content | Monomer Content | Monomer Content | Monomer Content |
|---|---|---|---|---|
| Storage | (%) 100 mg/mL | (%) 50 mg/mL | (%) 100 mg/mL | (%) 50 mg/mL |
| time (w) | Sorbitol/Potassium | Sorbitol/Potassium | Sorbitol/Sodium | Sorbitol/Sodium |
| 0 | 99.67 | 99.67 | 99.65 | 99.65 |
| 1 | 99.21 | 99.22 | 99.2 | 99.19 |
| 4 | 98.39 | 98.37 | 98.41 | 98.38 |
| 8 | 97.61 | 97.59 | 97.54 | 97.48 |
| 12 | 96.88 | 96.46 | 96.8 | 96.49 |

In summary, the addition of NaCl should be avoided in formulating adalimumab solutions at 50 mg/mL. If the presence of salts is favored, e.g., by reasons of osmolality—the sodium acetate has advantages over sodium chloride. Similarly, potassium based phosphate buffer systems equalled sodium phosphate buffer systems in terms of adalimumab stability.

In summary, a solution pH of 5.2 and the addition of 0.1% Tween 80 were favored over other alternatives for adalimumab solutions at about 50 mg/mL. Protein stability and particulate matter contamination after freeze/thaw studies and (accelerated) storage tests were used as evaluating criteria. Furthermore, polyols such as mannitol and sorbitol substantially contributed to protein stability with virtually identical potency. Preferential accumulation at the native state protein was not the only stabilization pathway, as both protein aggregation and fragmentation were impeded. NaCl impeded protein stability in the presence of polyols. The addition of sodium acetate did not deleteriously impact protein stability.

These data suggested a formulation comprising a potassium phosphate buffer, pH 5.2, 0.1% Tween 80 and ˜50 mg/mL mannitol or sorbitol—aiming at final osmolality values of ˜300 mosM/kg for an adalimumab concentration of 50 mg/mL.

### Example 2

### High Concentration Adalimumab Formulation

The following example provides the ingredients for a number of high concentration protein formulations comprising the ant-TNFα antibody adalimumab. Surprisingly, the formulations described below had a number of advantageous properties, despite the high concentration of antibody, i.e., about 100 mg/mL.

A number of characteristics of the formulations (referred to as F1 to F6) were studied relative to the commercial 50 mg/mL adalimumab formulation (F7), including turbidity. The turbidity of the solutions was determined by analysis of the undiluted solution. Turbidity is reported as NTU values (Nephelometric Turbidity Units).

Visible particle contamination was determined by visual inspection as described in German Drug Codex. Subvisible particles were monitored by the light obscuration method according to USP. Dynamic light scattering analysis of diluted solutions was employed to assess the hydrodynamic diameter (reported as the mean or Z-average size calculated by cumulants analysis of the DLS measured intensity autocorrelation function and polydispersity index, PDI, of the size distribution of particles).

The physicochemical stability of the formulations was assessed by SEC which allows detection of fragments and aggregates. To monitor chemical stability, SE-HPLC (detection of fragments and hydrolysis specimens) and CEX-HPLC (Cation Exchange HPLC) were used. CEX-HPLC resolves different lysine isoforms and degradation products (e.g., deamidated and oxidized species) that may have formed during storage.

The formulations tested are referenced as F1-F6 (Table 16), containing 100 mg/mL adalimumab in different matrices spanning from pH 5.2 to pH 6.0, formulated with different polyols and with or without sodium chloride.

TABLE 16
Components Of Adalimumab Formulations F1-F7.

| Component | F1 | F2 | F3 | F4 | F5 | F6 | F7 |
|---|---|---|---|---|---|---|---|
| Adalimumab | 100 | 100 | 100 | 100 | 100 | 100 | 50 |
| Mannitol | 12 | 42 | — | 12 | 42 | — | 12 |
| Sorbitol | — | — | 42 | — | — | 42 | — |
| Polysorbate 80 | 1 | 1 | 1 | 1 | 1 | 1 | 1 |
| citric acid * H₂O | 1.305 | 1.305 | 1.305 | 1.305 | 1.305 | 1.305 | 1.305 |
| Sodium citrate | 0.305 | 0.305 | 0.305 | 0.305 | 0.305 | 0.305 | 0.305 |
| dehydrate | | | | | | | |
| Na₂HPO₄ * 2 H₂O | 1.53 | 1.53 | 1.53 | 1.53 | 1.53 | 1.53 | 1.53 |
| NaH₂PO₄ * 2 H₂O | 0.86 | 0.86 | 0.86 | 0.86 | 0.86 | 0.86 | 0.86 |
| NaCl | 6.165 | 0 | 0 | 6.165 | 0 | 0 | 6.165 |
| NaOH | q.s | q.s | q.s | q.s | q.s | q.s | q.s |
| target pH | 5.2 | 5.2 | 5.2 | 6.0 | 6.0 | 6.0 | 5.2 |

The above 100 mg/mL formulations (F1-F7) were further studied to characterize overall stability and viscosity, as described below in Examples 3-6.

The following is a description of how to make high concentration adalimumab formulations, particularly with respect to exemplary solutions F2 and F6. The starting solution is a solution of purified antibody at low concentration (lower than the high concentrations of the invention) in a liquid buffer, for example in a buffer resulting from the preceding manufacturing process step. In this case, adalimumab solution was provided at a concentration of about 70 mg/mL in a buffer system identical to F7 without surfactant at pH 5.2. The starting solution is then concentrated and diafiltered by ultrafiltration, preferably in a tangential-flow filtration system, using a membrane able to retain quantitatively the antibody, for example with a cutoff of 10 kD.

As an example, the representative formulations F2 and F6 were manufactured by diluting the concentrate to about 50 mg/L using the corresponding matrix without surfactant as diafiltration buffer. A continuous buffer exchange was conducted using the tangential-flow filtration system. The diafiltration was generally carried out at constant retentate volume, with at least 5 volumes, or preferably 8 volumes, of diafiltration buffer. In a last step, the diafiltered solution was further concentrated to a high concentration, for example higher or equal to 150 mg/mL. The final turbid retentate was then recovered out of the ultrafiltration system by flushing the tubes with diafiltration buffer. After the addition of the respective amount of polysorbate 80 and adjusting to the target protein concentration using diafiltration buffer, a high concentration liquid formulation was obtained, which was clear to slightly opalescent. After filtration through a 0.22 μm filter, the solution was stable for at least about 12 months if stored at about 2-8° C.

### Example 3

### Stability of High Concentration Adalimumab Formulation Against Freeze/Thaw Stress

In order to demonstrate that adalimumab formulations are stable at 100 mg/mL protein concentrations, freeze/thaw stress (freezing performed at −80° C., thawing performed at 25° C.) experiments were carried out.

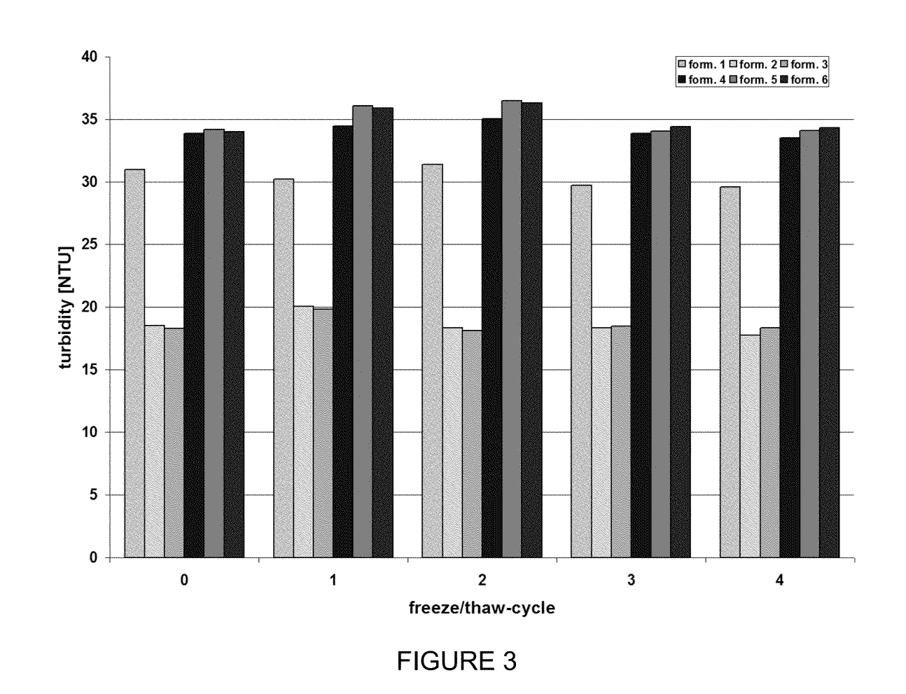

An array of analytical methods sensitive to particle formation was used to detect potential physical instabilities. Turbidity was measured as an indicator of the development of particle aggregates in the colloidal or in the visible range. The turbidity (reported as NTU values) did not change significantly even after the fourth cycle of freeze/thaw (FIG. 3). Increased turbidity of solutions of higher pH may be attributed to increased protein-protein interactions due to lowered charge repulsion at the pH approaching the pI of the protein (adalimumab 8.5) (Wang et al. (2007) J Pharm Sci 96 (1) 2457-2468).

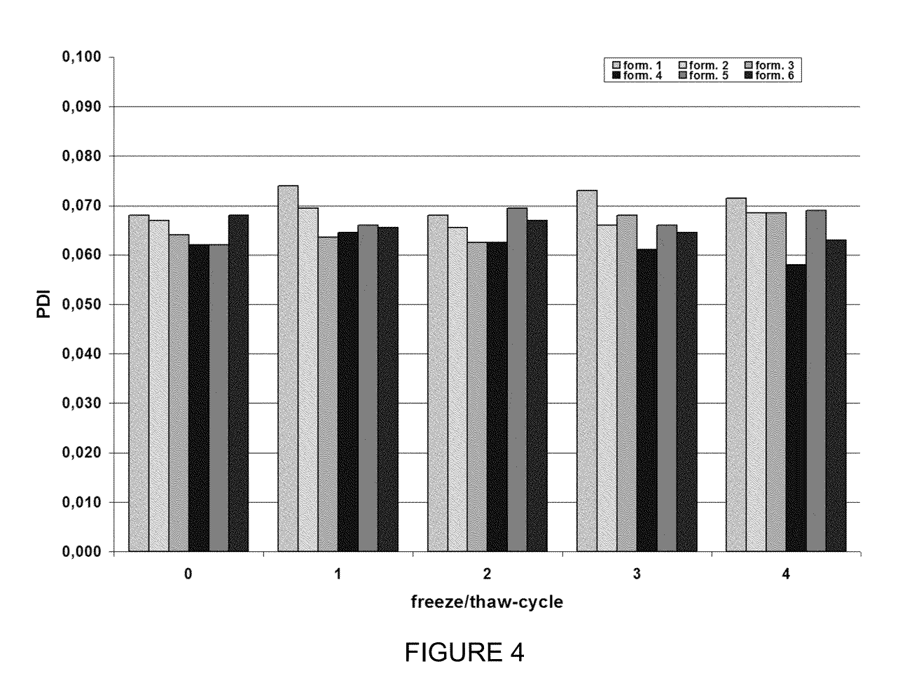

Dynamic light scattering was employed as a method for determining particle size in the submicron range. The polydispersity index value obtained in the course of the size distribution determination was used as another sensitive indicator of aggregation in the colloidal or in the micrometer size range. Similar to the turbidity data, none of the tested formulations showed any signs of physical instability (FIG. 4).

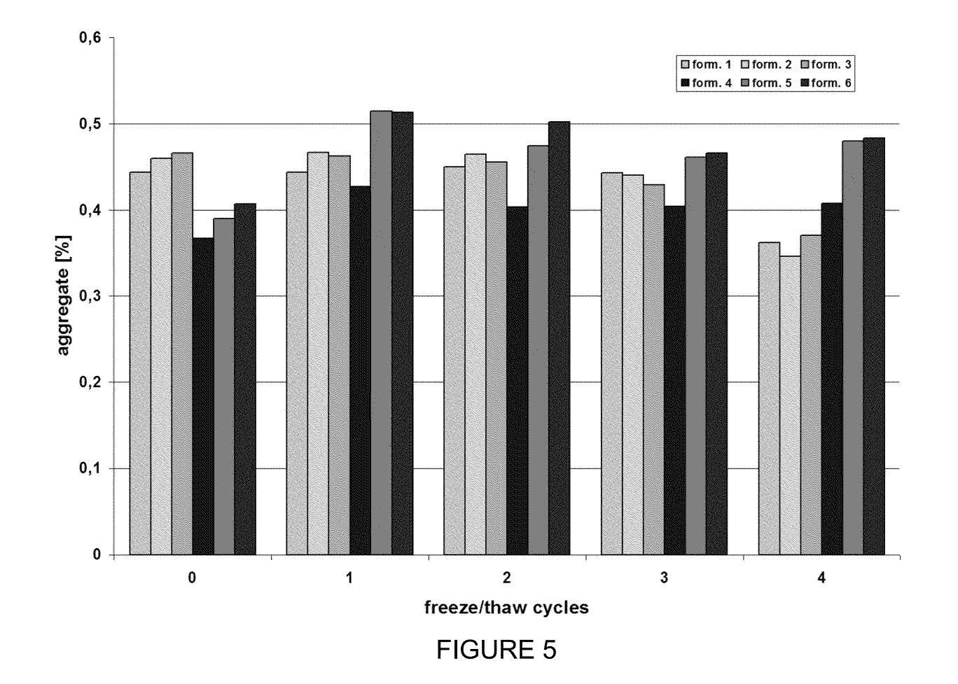

In addition, size exclusion data was evaluated. FIG. 5 depicts aggregate levels. No signs of physico-chemical instabilities were detected in relation to the repeated freeze/thawing stress.

It is well known that freeze/thaw processing can result in substantial protein denaturation and aggregation, resulting in soluble and insoluble aggregate formation (Parborji et al. (1994) Pharm Res 11 (5)764-771). All of the formulations presented herein were subjected to repeated freeze thaw processing and the results demonstrated that none of the formulations were sensitive to repeated freeze/thaw cycles (−80° C./25° C.). All of the formulations were similarly stable independent of their pH (in all cases there was no significant change as compared to initial values) despite the higher pH of the formulations which were closer to the pI of adalimumab (i.e., 8.5).

Data from a separate study comparing different buffer solutions confirmed these results. The most beneficial buffer system with regard to a homogeneous solution (i.e. a solution with the least gradient in pH, osmolality, density) after freeze-thaw and the least pH-shift during freeze-thaw proved to be a buffer composition with no NaCl added (see Example 1). NaCl-free buffer systems formulated at pH 6 proved to have the least pH-shift of all the pH levels evaluated.

### Example 4

### Stability of 100 mg/mL Formulations Containing Different Polyols as Isotonizer

Differential Scanning calorimetry (DSC) was employed to test all of the 100 mg/mL adalimumab formulations for generally stability. DSC data were obtained using a VP Capillary DSC form Microcal. All experiments were performed with 1 heating run using the following standardized procedure: temp range: 20° C.-90° C., heating rate: 1 K/min, protein concentration 1 mg/mL).

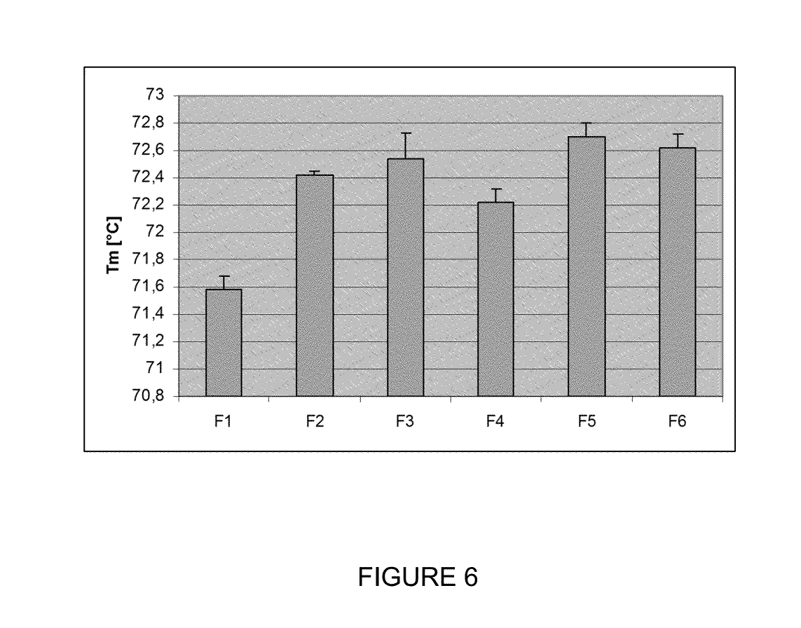

Higher Tm values are generally indicative of increased conformational stability (Singh et al. (2003) AAPS PharmSciTech 4 (3) article 42). FIG. 6 provides Tm values for the 100 mg/mL adalimumab formulations. These data showed that all formulations achieve high Tm values. However, the sodium chloride free formulations (F2, F3, F5, F6) showed significantly increased Tm values indicating the robustness of these formulations. Since formulations are tested at 1 mg/mL, the Tm data of F1 is the same as the Tm of F7, thereby confirming the improved stability of the 100 mg/mL formulations without sodium chloride or at pH 6.0 over the F7 formulation.

A stir stress model using magnetic stir bars was used to detect physico-chemical instabilities of the new adalimumab formulations. This well known model induces stress by subjecting adalimumab to long term air-liquid interface exposition as well as stirring related cavitation which leads to formation of soluble and insoluble protein aggregates in a predictable manner.

Generally, proteins formulated at pH values in the range of their respective pI (adalimumab pI 8.5, low net charge, minimized electrostatic repulsive forces) are more susceptible to air-liquid interface related aggregation due to reduced repulsive forces. Additionally, ionic excipients, such as sodium chloride, play a role in protein aggregation due to their ionic shielding properties. Hydrophobic attractive forces may be reduced with the presence of sodium chloride thereby reducing protein-protein interactions and increasing the colloidal stability (Shire et al. (2004) J Pharm Sci, 93 (6)1390-1402).

Turbidity data were evaluated to detect aggregate formation induced by stir stress. Table 17 depicts nephelometric values in relation to the formulation composition and stirring time. Initial turbidity values for F1-F3 (formulated at lower pH of 5.2) demonstrated differences between sodium chloride containing (F1) and NaCl free (F2, F3) solutions. In contrast, solutions adjusted to a higher pH of 6.0 (F4-F6) were characterized by higher turbidity. It is known in the art that NaCl may reduce the clarity of mAb solutions after mechanical stress such as stirring (e.g., Fesinmeyer et al. (2009) Pharm Res, 26 (4)903-913).

TABLE 17
Turbidity (NTU) Vs. Stirring Time Of Formulations F1-F6.

| | T0h | T1h | T5h | T24h | T48h |
|---|---|---|---|---|---|
| F1 | 31.5 | 33.25 | 36.05 | 46.9 | 54.85 |
| F2 | 19.8 | 20.25 | 23.1 | 28.65 | 40 |
| F3 | 18.8 | 19.75 | 22.2 | 27.3 | 39.5 |
| F4 | 36.8 | 37.25 | 42.4 | 63.45 | 86.75 |
| F5 | 36.1 | 38.85 | 44.5 | 64.3 | 76.7 |
| F6 | 36.6 | 38.85 | 42.8 | 59.1 | 72.7 |

Stirring for up to 48 hours induced increased turbidity values in all tested formulations. NaCl-free formulations at a lower pH were the least prone to turbidity increase by stirring. Surprisingly, all tested 100 mg/mL formulations tested exhibited significantly reduced turbidity after stirring compared to lower concentration (50 mg/mL) adalimumab formulation. (Table 18).

Generally, AN opalescent appearance is a simple consequence of Rayleigh scatter and linearly related to protein concentration. However, opalescent appearance does not result in physical instability (Sukumar et al. (2004) Pharm Res 21 (7)1087-1093). The 50 mg/mL adalimumab formulation showed turbidity of 63-130 NTU after 24 hours stirring and 109-243 NTU after 48 hours, whereas the 100 mg/mL formulations of adalimumab resulted in values ranging between 27-63 (24 hours) and 40-87 (48 hours). According to Treuheit et al. ((2002) Pharm Res 19 (4)511-516), increased protein concentration reduces air-liquid interface induced aggregation in OPC-Fc solution in a range lower than 10 mg/mL. Similar results have been reported by Kiese et al. ((2008) J Pharm Sci 97 (10)4347-4366). Unexpectedly, the new adalimumab formulations were characterized by increased stir stress stability in the much higher protein concentration range of 100 mg/mL.

Therefore, the new formulations have increased stability compared to the 50 mg/mL formulation.

TABLE 18
Data From Stir Stress Experiments Conducted Using Different
Lots Of 50 mg/mL Adalimumab Formulations (F7).

| | lot | lot | lot | lot | lot | lot |
|---|---|---|---|---|---|---|
| | 201359A | 191299A | 221479A) | 221489A | 241679A | 231649A |
| NTU T24 | 63.3 | 130.4 | 94.8 | 92.1 | 82.0 | 88.0 |
| | (22.85) | (39.24) | (28.98) | (30.88) | (29.75) | (30.15) |
| NTU T48 | 109 | 243 | n.a. | 178.4 | 136 | 175.7 |
| | (52.50) | 84.23) | | (55.80) | (30.65) | (63.37) |

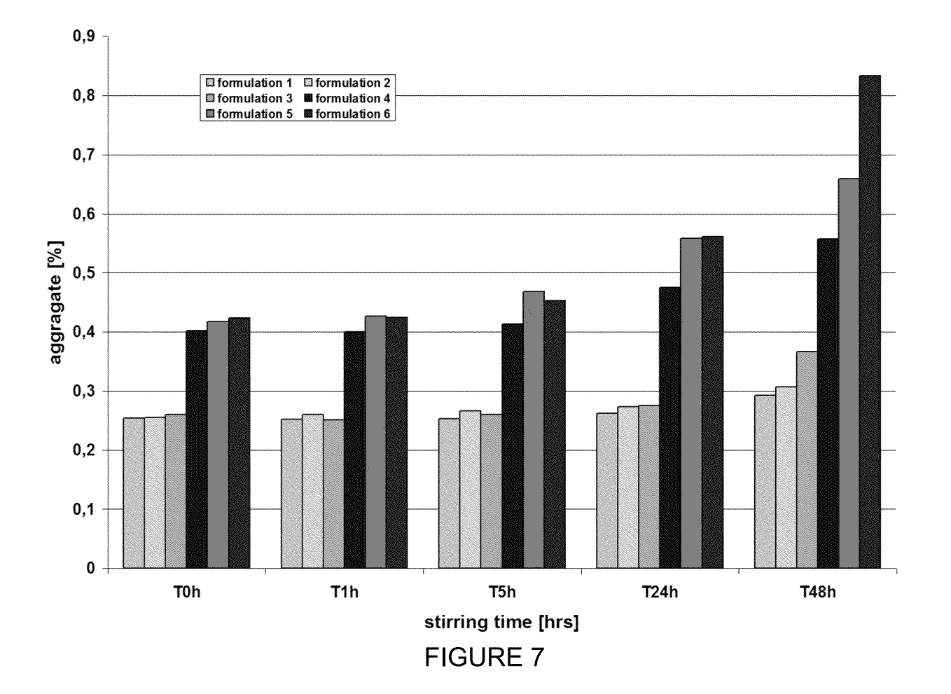

Additionally, size exclusion chromatography data revealed that all 100 mg/mL formulations had aggregate levels <1% after 48 hours of stirring, supporting the claim of stability of the new formulations (FIG. 7). Lower pH and absence of sodium chloride were again beneficial. This data verifies the surprising finding that the new formulations are stable despite pH values approaching the pI of adalimumab, and that absence of NaCl is beneficial, although a low net charge at higher pH is generally believed to add to instability.

### Example 5

### Long Term Stability of 100 mg/mL Adalimumab Formulations with and without Sodium Chloride, pH 5.2 and 6.0, 2 Different Polyols

The new 100 mg/mL adalimumab formulations were subjected to long term storage to verify superior stability compared to the 50 mg/mL standard formulation. Stability data over 12 months at 5° C. (recommended storage temperature for the commercial product) were evaluated. The data indeed suggest that the new formulations displayed no reduced stability (Table 19).

Regarding SEC and IEX, no significant loss in monomer content or measurable degradation occurred.

Furthermore, despite the higher protein concentration of the new adalimumab formulations, significant enhancements in terms of particle contamination in the subvisible range compared to 12 M data of 50 mg/mL marketed adalimumab formulation were obtained. Testing for subvisible particulate contamination (indicating aggregation, precipitation and general physical instability phenomena) revealed that the new adalimumab formulations remained practically free from subvisible particles. Initial particles of max 28 (>=10) and max 3 (>=25) were significantly lower than for the 50 mg/mL formulation F7 (703 and 38, respectively)

Additionally, particle levels did not change significantly throughout the 12 months stability testing and remained at significantly lower levels than F7.

The drug product batches were virtually equivalent with regards to their physicochemical stability at all storage conditions tested. This is surprising, as it is well accepted that, e.g., physical stability tends to decrease at higher protein concentrations (Wang W. (1999) Int J Pharm 185:129-188).

TABLE 19
Comparison Of Analytical Data Of Stability
Studies Of F1-F7 (T0/12 M).

| | F1 | F2 | F3 | F4 | F5 | F6 | F7 |
|---|---|---|---|---|---|---|---|
| SEC Monomer | 99.6 | 99.0 | 99.7 | 99.4 | 98.7 | 99.4 | 99.8 |
| | 99.4 | 99.4 | 99.4 | 99.2 | 99.1 | 99.1 | 99.4 |
| IEX Sum of | 85.9 | 85.7 | 85.9 | 86.0 | 85.8 | 86.0 | 85.1 |
| lysin var | 83.5 | 83.2 | 83.2 | 84.9 | 84.7 | 84.6 | 82.6 |
| Clarity | 29.3 | 16.10 | 16.5 | 32.20 | 31.5 | 32.6 | 19.7 |
| | 30.2 | 17.10 | 17.85 | 34.0 | 33.5 | 33.9 | 18.4 |
| DAC score | 0.0 | 0.0 | 0.0 | 0.0 | 0.0 | 0.0 | 0.4 |
| | 0.1 | 0.1 | 0.4 | 0.0 | 0.0 | 0.0 | 0.0 |
| Sub vis >=10 | 31 | 4 | 2 | 6 | 18 | 28 | 703 |
| | 2 | 4 | 3 | 7 | 8 | 5 | 746 |
| Sub vis >=25 | 0 | 0 | 0 | 0 | 0 | 1 | 38 |
| | 1 | 0 | 1 | 1 | 3 | 2 | 36 |

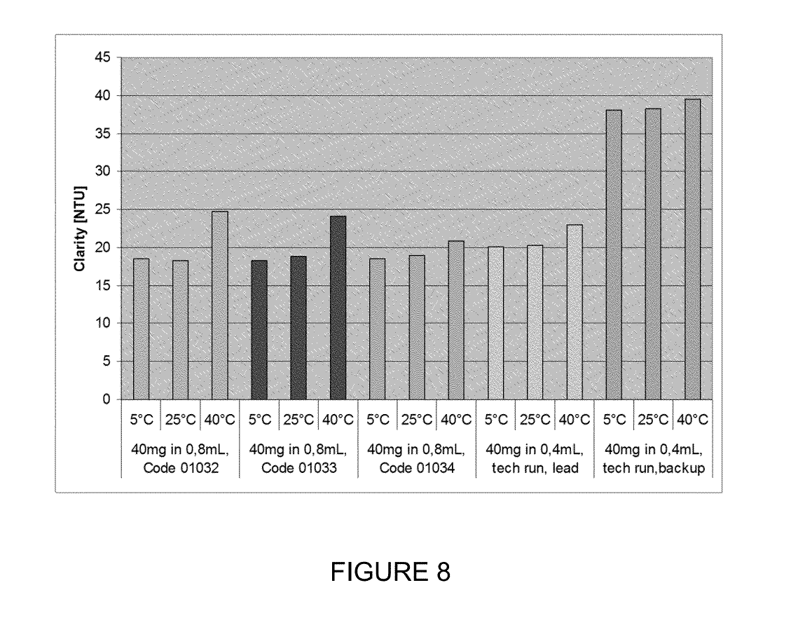

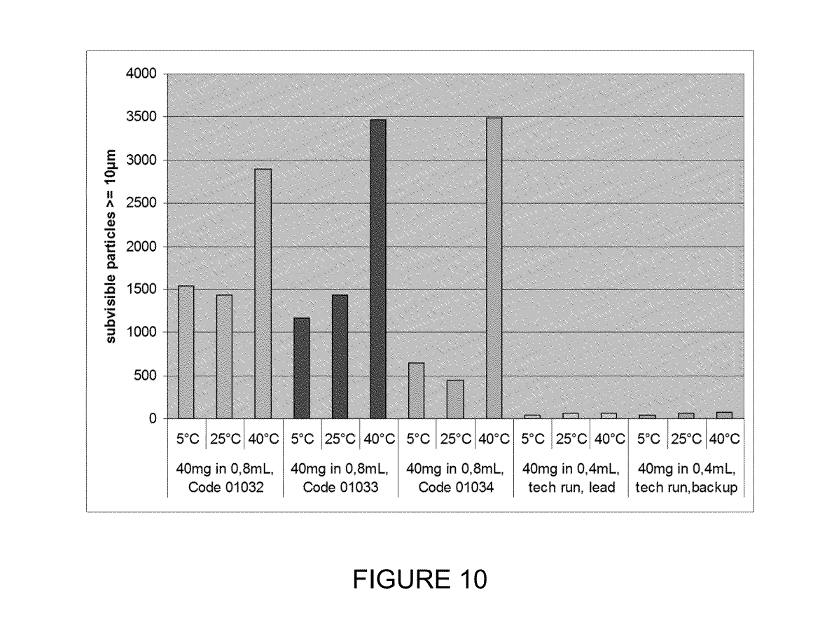

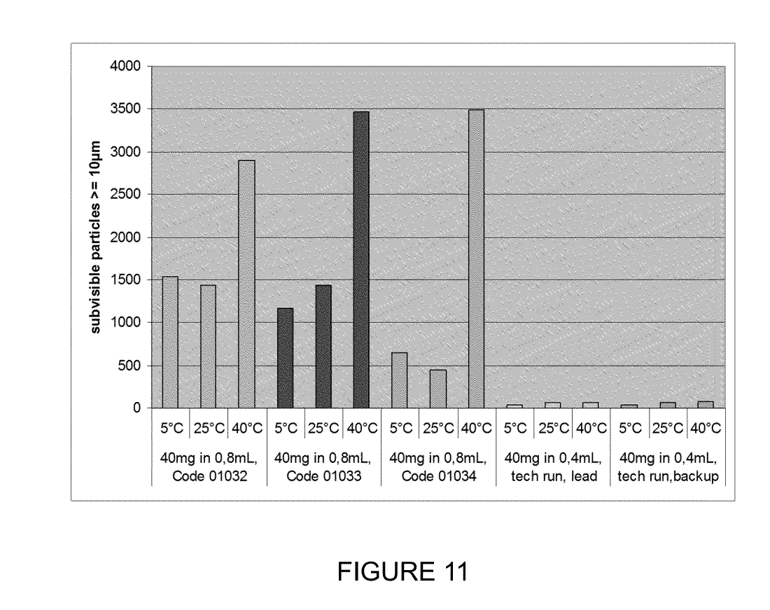

To verify the results of increased storage stability of the new 100 mg/mL formulations, 2 representative formulations, F2 and F6, were subjected to accelerated stability testing (3 months at 5°, 25°, 40° C.) and compared with the marketed 50 mg/mL formulation (representative batches from registration runs). The results of these experiments are summarized in FIGS. 8-13.

Turbidity data from these batches verifies the superior behavior of the NaCl free formulations at 100 mg/mL, especially at the lower pH of 5.2. Increasing the concentration of protein in solution is generally known to increase opalescence and thereby the turbidity readout due to Rayleigh scattering (Sukumar et al. (2004) Pharm Res 21 (7)1087-1093). Surprisingly, the new formulations without sodium chloride revealed similar turbidity levels at the same pH of the 50 mg/mL formulations (FIG. 8).

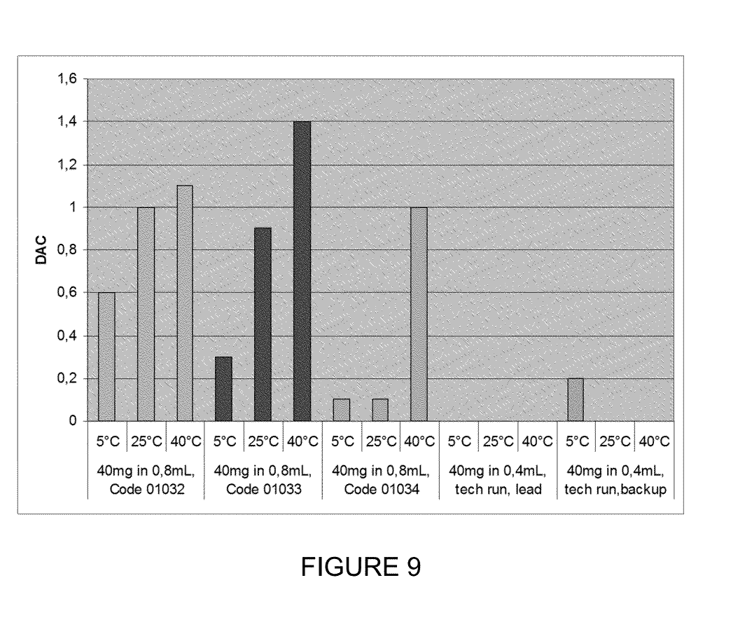

FIGS. 9-11 provide detailed data of particulate formation (visible and subvisible particles) of the new formulations. The surprising finding of increased stability was verified. In fact, it was possible to reduce the both subvisible and visible particle score even after 3 months storage at elevated temperature.

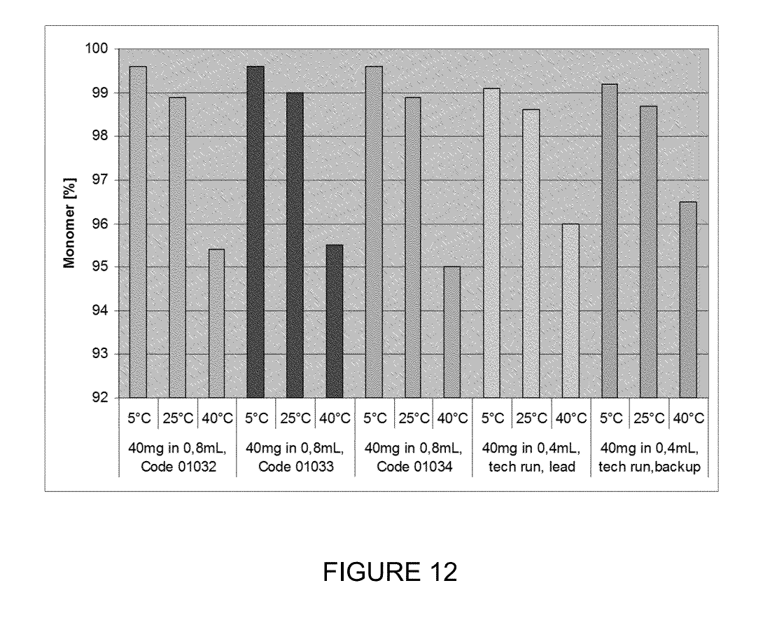

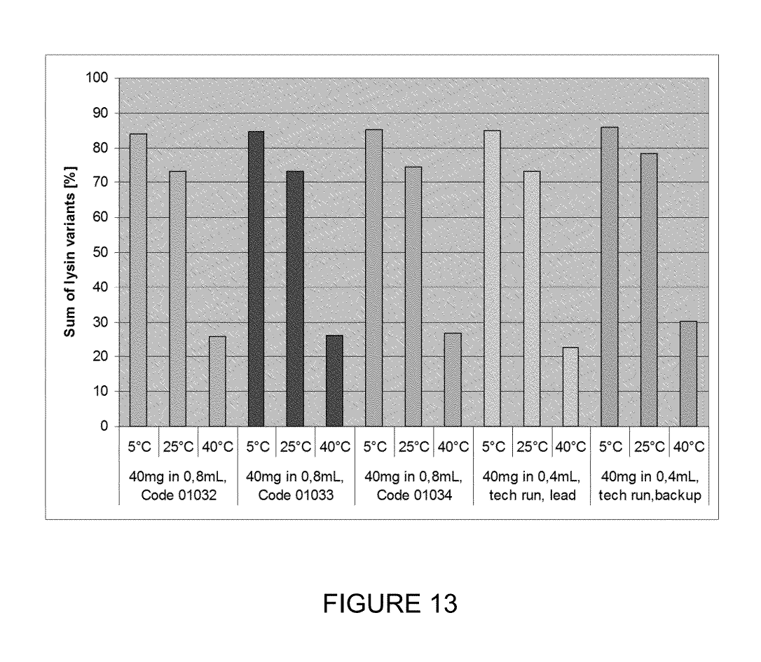

Data provided in FIG. 12-13 further verified the stability of the 100 mg/mL formulations as it does not reveal any stability issues for both SEC analytics and chemical stability tested using IEX.

### Example 6

### Increased Manufacturability of 100 mg/mL Adalimumab Formulations Compared to 50 mg/mL Adalimumab Formulations

This example summarizes data related to improved process stability of the new 100 mg/mL adalimumab formulations (representative formulations F2 and F6) compared to the currently marketed 50 mg/mL product.

Mechanical stress generated by pumping, filtration, mixing, fill-finish processes, shipping or shaking may cause denaturation and consecutively aggregation due to exposure of the protein to air-water interfaces, material surfaces and shear forces (Mahler at al. (2005) Eur Pharm Biopharm 59:407-417; Shire et al. (2004) J Pharm Sci, 93 (6)1390-1402).

Viscosity values were determined initially as a basic parameter characterizing the processability of protein solutions. Table 20 provides viscosity data obtained for the F1-F7 formulations. Increasing protein concentration led to increased viscosities compared to the 50 mg/mL formulation (F7).

Removal of the electrostatically shielding agent NaCl is expected to increase hydrophobic protein interactions, especially at pH values approaching the pI of adalimumab, thereby increasing the viscosity. This effect was reported to be most pronounced at NaCl concentration <200 mM (Shire et al. (2004) J Pharm Sci, 93 (6)1390-1402).

Unexpectedly, however, removal of NaCl (F1 contains ˜105 mM NaCl) from the formulations resulted in still relatively low viscosity values of about 3.1-3.3 mPas*s (F2, F3, F5, and F6). This was especially surprising for the solutions at a higher pH value of 6.0 (F5, and F6).

In summary, all formulations are characterized by viscosities in a range optimal for liquid fill-finish manufacturing operations.

TABLE 20
Comparison Of Viscosities
At 25° C. of F1-F7.

| | Viscosity |
|---|---|
| Formulation | [mPa*s] |
| F1 | 2.8902 |
| F2 | 3.1278 |
| F3 | 3.1223 |
| F4 | 2.9018 |
| F5 | 3.2585 |
| F6 | 3.2279 |
| F7 | 1.3853 |

In a lab model mimicking the stress induced by sterile filtration in the course of the aseptic manufacturing process, two representative new formulations containing 100 mg/mL adalimumab provided analytical data showing that all formulations were stable against filtration related shear stress. DLS data did not show any signs of the development of higher molecular weight aggregates, since the polydispersity index, a sensitive indicator for low levels of higher molecular weight sub-populations did not increase significantly. DLS measurements are specifically used to detect low amounts of higher molecular weight species, e.g. aggregates, in a size distribution, since those species possess higher scattering intensity (proportional to d6) and thereby will influence ZAve and polydispersity index as an indicator of the ZAve size distribution significantly. Additionally, SEC data verified no induction of aggregation by filtration.

Surprisingly, even the 100 mg/mL formulations did not reveal any instability. Even after multiple sterile filtrations as a worst case scenario processability was maintained at a high level despite increased protein content.

TABLE 21
DLS And SEC Data Comparing F2, F6 And F7 In Terms Of
Stability Against Sterile Filtration Stress.

| Method | F2, 100 mg/mL | F6, 100 mg/mL | F7, 50 mg/mL |
|---|---|---|---|
| DLS (nm) | | | |
| PDI before | 0.058 | 0.054 | 0.022 |
| filtration | | | |
| PDI after 5 | 0.057 | 0.050 | 0.032 |
| filtration cycles | | | |
| SEC (% | | | |
| aggregates) | | | |
| Before | 0.235 | 0.429 | 0.220 |
| filtration | | | |
| After 5 | 0.238 | 0.426 | 0.310 |
| filtration cycles | | | |

To further demonstrate the high stability of the new adalimumab formulations against process related stress, formulations were tested in a stir stress model comparing their behavior against different stirring speeds of a magnetic stir bar (stir stress occurs under production conditions in the compounding process step).

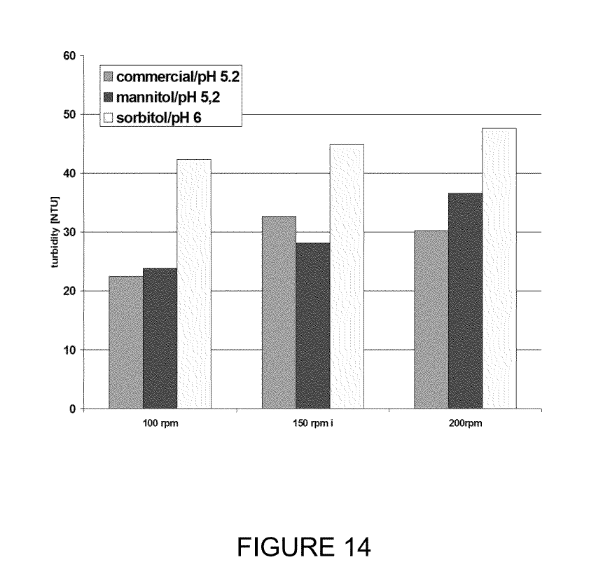

The comparison of stir stress resistance revealed no increase in turbidity at 100 mg/mL protein concentration (FIG. 14). Both representative 100 mg/mL formulations without sodium chloride and increased polyol content behaved similarly to the commercial formulation at pH 5.2 at all tested stirring speeds. At higher stirring speeds, all formulations showed slightly increased turbidity values after 24 hours of stiffing, however, no notably increased susceptibility to instability due to shear stress at 100 mg/mL was detected.

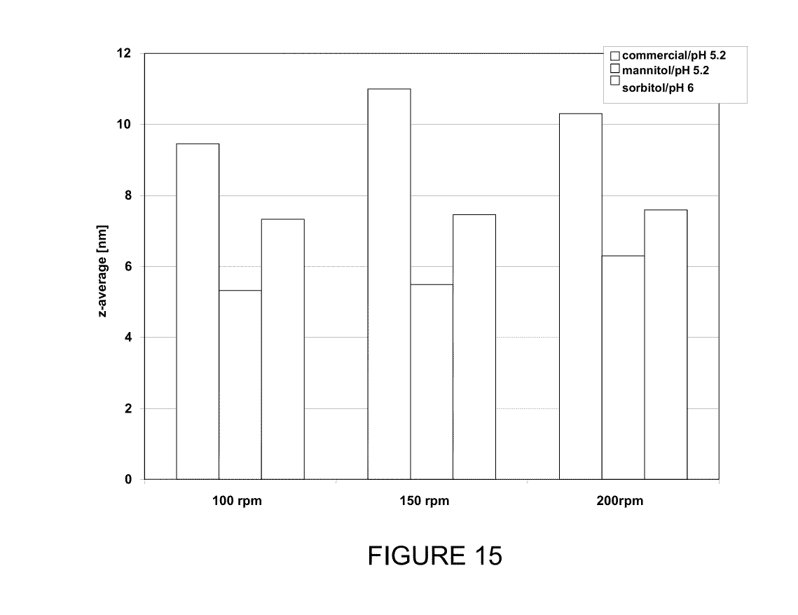

A comparison of the change of the hydrodynamic diameters as obtained by DLS measurement resulted in similar data. Both 100 mg/mL formulations behaved similarly to the 50 mg/mL formulation, even though formulations with higher protein concentrations are believed to be more sensitive to stir stress. Surprisingly, formulation F2 with the highest pH revealed the lowest relative increase in both turbidity and hydrodynamic diameter analytics (FIG. 15).

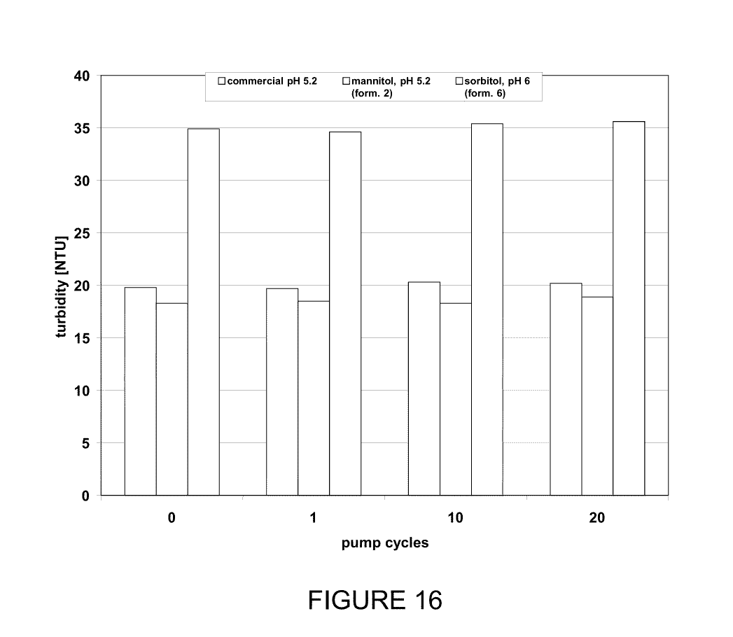

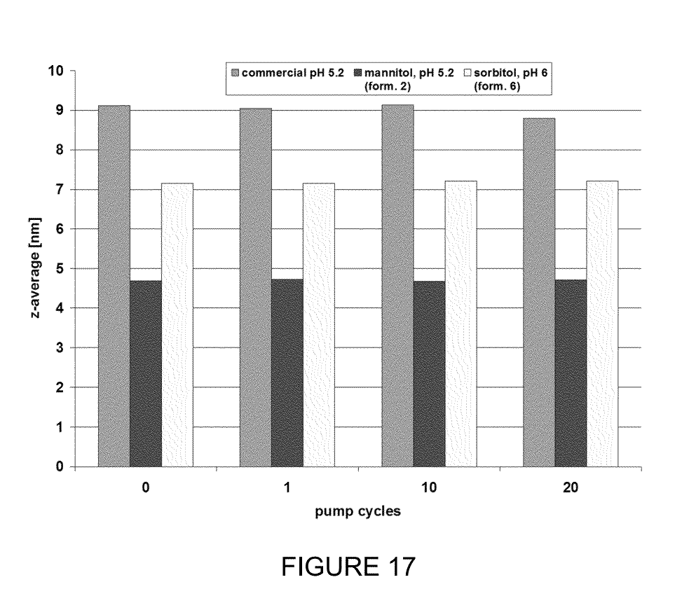

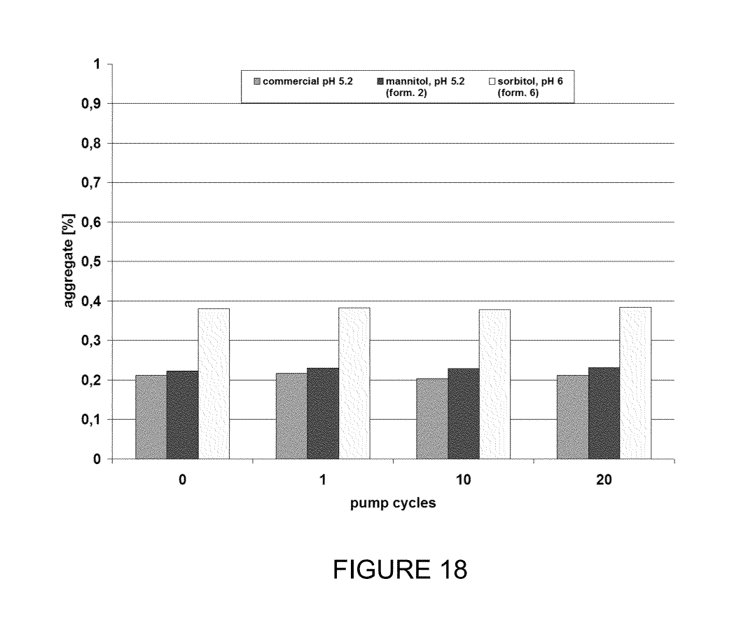

This surprising finding of similar process stability even at higher protein concentration was further confirmed by a mechanical stress model mimicking the stress induced by the pumping process. This last step of the manufacturing process encompasses shear stress by peristaltic pumping, thereby increasing the risk of solution instabilities. Again, data obtained using turbidity (FIG. 16) and DLS (FIG. 17, Table 22) confirmed that the new 100 mg/mL formulations do not undergo particle development reactions, and remained similarly stable as the 50 mg/mL formulation. No susceptibility to pump stress induced aggregate formation was detectable. This finding was additionally confirmed by SEC data, which did not reveal any differences of the tested formulations in relation to the pump cycles (FIG. 18).

TABLE 22
DLS Data (PDI) Comparing F2, F6 And F7 Stability
Before And After Several Pump Cycles.

| Pump | Commercial | Mannitol, | Sorbitol, |
|---|---|---|---|
| Cycles | pH 5.2 | pH 5.2 (form. 2) | pH 6 (form. 6) |
| 0 | 0.06 | 0.055 | 0.028 |
| 1 | 0.059 | 0.064 | 0.029 |
| 10 | 0.061 | 0.058 | 0.032 |
| 20 | 0.059 | 0.069 | 0.022 |

Using a variety of filling equipment (rotary piston and peristaltic pumps), differences in stability of 100 mg/mL formulations were evaluated.

These studies showed that the higher shear stress generated in piston pumps led to increased visible particle counts, especially for sodium chloride containing formulations at higher pH (F1 and F4). Similar results have recently been reported from Bausch, Ursula J. (Impact of filling processes on protein solutions. 2008, PhD Thesis, University of Basel, Faculty of Science; http://edoc.unibas.ch/845/1/DissB_{—}8427.pdf), but only at protein concentrations of rituximab solutions of 10 mg/mL. Surprisingly, sodium chloride formulations with 100 mg/mL adalimumab displayed improved process ability under high shear conditions using piston pumps.

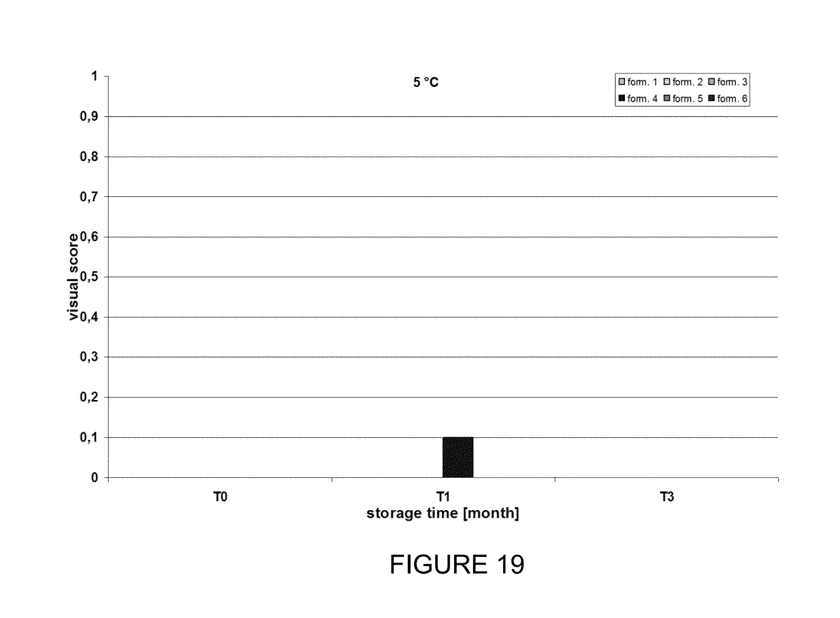

FIGS. 19-22 provide particle counts and turbidity data verifying increased sensitivity of NaCl-containing adalimumab solutions to increased process stress conditions: Determination of particle size ranges >=10 μm and >=25 μm according to the DAC visual score method are an essential quality attribute for parenteral drugs. Therefore, reduction in subvisible particles in the NaCl-free formulations provides a significant formulation improvement.

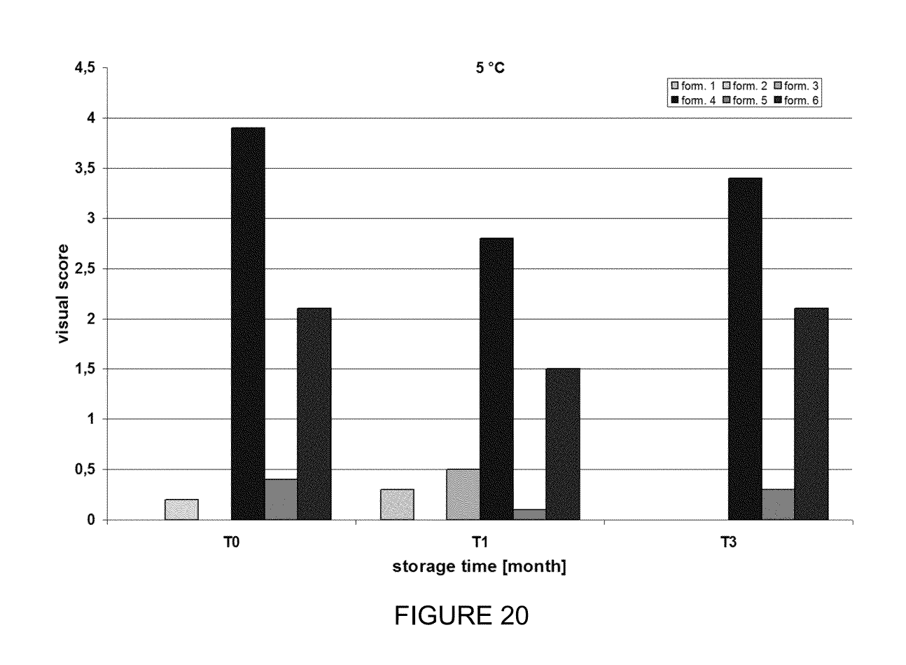

As depicted in FIG. 19, peristaltic filling did not result in visible particle generation directly after filling (T0) and after storage. In contrast, piston filling resulted in significant particle counts even at T0 for the solutions formulated at pH 6.0 (FIG. 20). The highest values were measured in F4, containing sodium chloride, whereas F5-F6 resulted in significant lower scores, verifying the improved stability of sodium chloride free formulations against process stress.

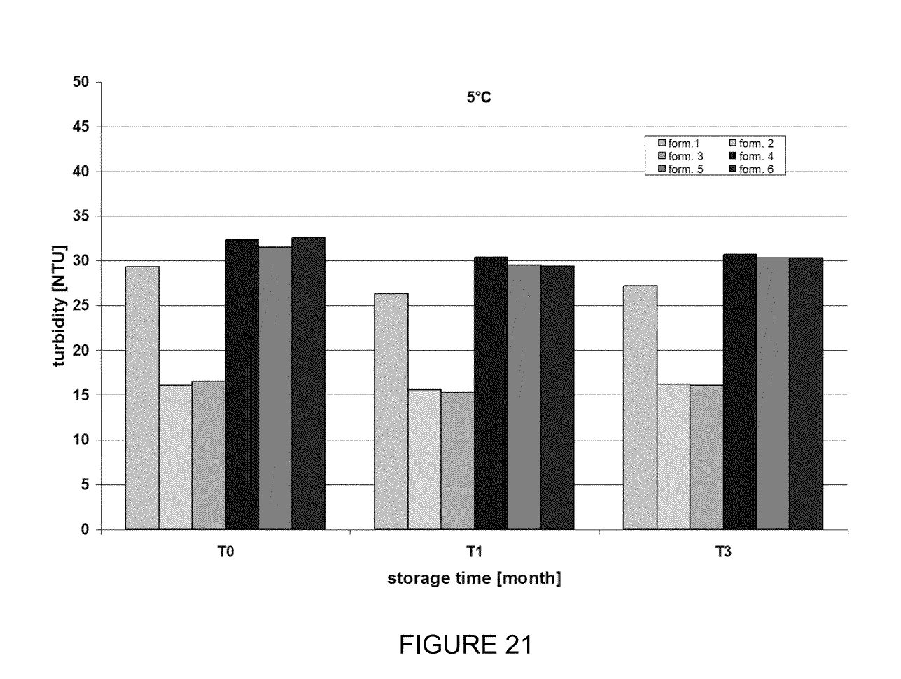

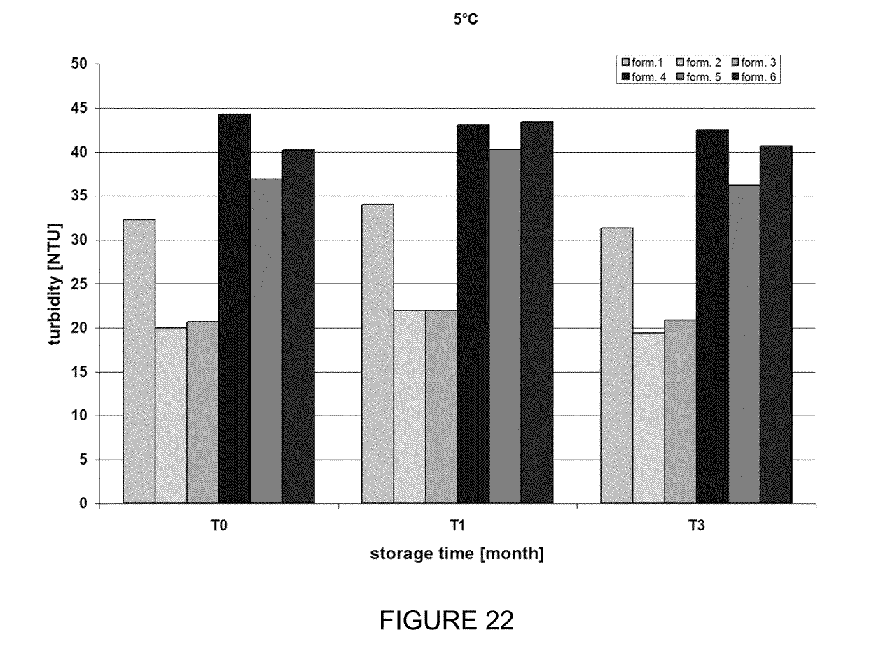

Supporting results were obtained by turbidity measurements (FIGS. 21-22). Initial values of solutions filled using the piston pump were higher than those filled using the peristaltic filling process. Sodium chloride free formulations resulted in lowered turbidity than those containing sodium chloride. In addition, shear stress by piston filling allowed for a differentiation of F4 (with sodium chloride) from F5 and F6 (without sodium chloride) in terms of turbidity.

### Example 7

### Comparison of Different Polyol Concentrations in Sodium Chloride Free Formulations

The following sodium chloride-free formulations containing 100 mg/mL adalimumab were tested for the influence of the polyol concentration of short term stability at 5° C. Formulations were adjusted to pH 6.0 to represent poor conditions in terms of aggregation and particle formation tendency.

TABLE 23
Overview Of Formulations Tested In Example 6.

| | F8 | F9 | F10 | F11 |
|---|---|---|---|---|
| | #1 | #2 | #3 | #4 |
| | Manitol | Manitol | Sorbitol | Sorbitol |
| Component | (12 mg/mL) | (42 mg/mL) | (12 mg/mL) | (42 mg/mL) |
| Adalimumab | 100 | 100 | 100 | 100 |
| Mannitol | 12 | 42 | — | — |
| Sorbitol | — | — | 12 | 42 |
| Tween 80 | 1 | 1 | 1 | 1 |
| citric acid * H₂O | 1.305 | 1.305 | 1.305 | 1.305 |
| Sodium citrate * 2 | 0.305 | 0.305 | 0.305 | 0.305 |
| H²O | | | | |
| Na₂HPO₄ * 2 H₂O | 1.53 | 1.53 | 1.53 | 1.53 |
| NaH₂PO₄ * 2 H₂O | 0.86 | 0.86 | 0.86 | 0.86 |
| NaCl | 0 | 0 | 0 | 0 |
| NaOH | q.s | q.s | q.s | q.s |
| target pH | 6.0 | 6.0 | 6.0 | 6.0 |

Mannitol or sorbitol was used at a concentration of 42 mg/mL to meet tonicity requirements of sodium chloride-free solutions. Data showed that in comparison to a formerly used concentration of 12 mg/mL, both polyols not only contributed to the osmolality of the solutions, but additionally had a significant impact on protein stability.

Stability data suggested improved clarity for higher polyol concentrations, independent of the type of the polyol. Under conditions that are generally rated as not optimal (e.g., pH 6.0 close to the pI of adalimumab), formulations with higher polyol concentrations showed improved clarity even after short storage of 4 weeks at 5° C. This was observed with several analytical methods.

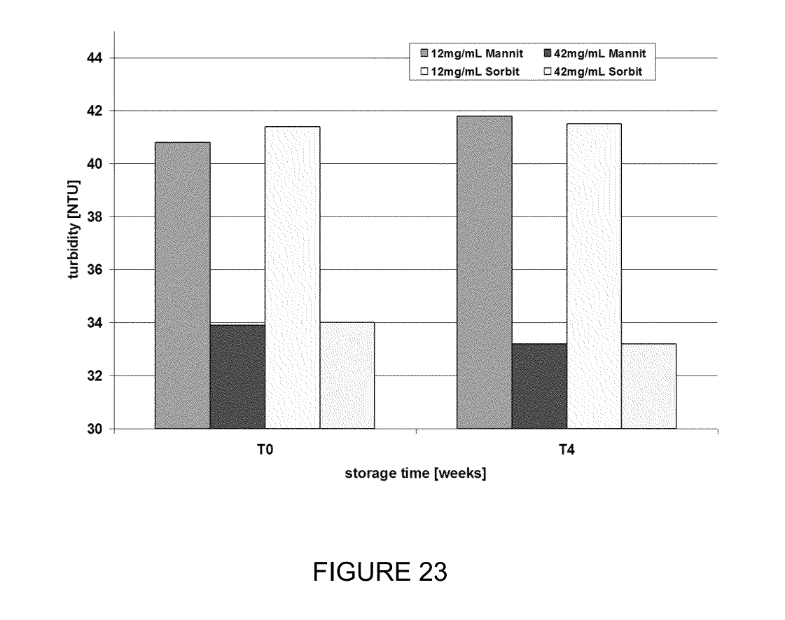

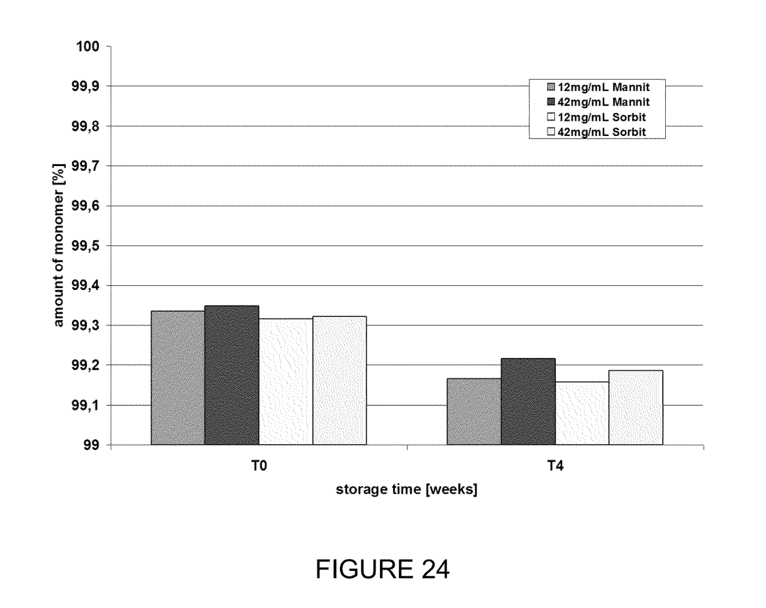

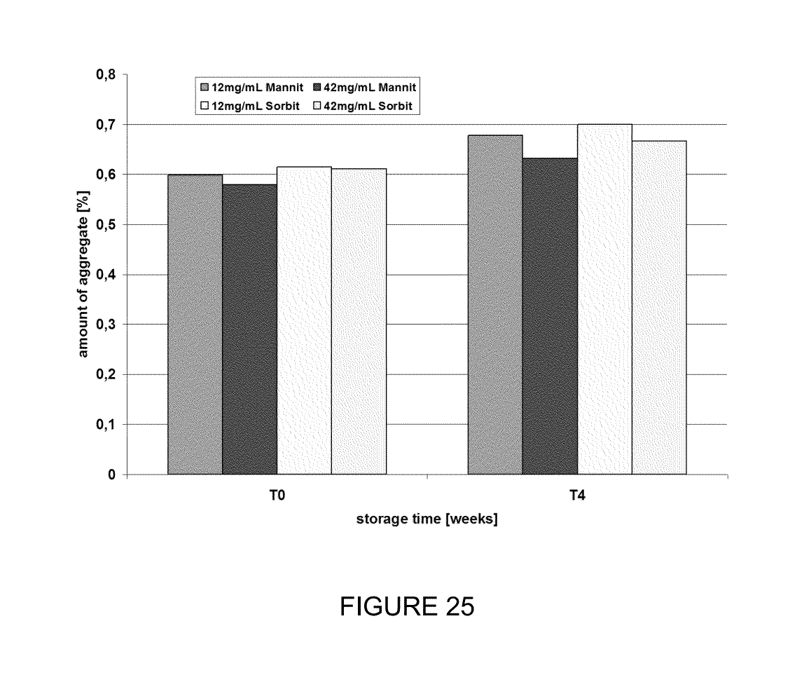

FIG. 23 reveals that clarity of the tested formulations was significantly reduced by increasing the polyol concentration and could be kept at lower levels over the tested period. Additionally, after 4 weeks at 5° C. slight reduction of aggregation resulting in higher monomer content at higher polyol concentartions was observed (FIGS. 24 and 25). Subvisible particles in the range of >=10 μm were reduced (e.g., at T0) at higher polyol concentrations.

### Example 8

### Stable High Protein Concentration Formulations of Human Anti-TNF-Alpha□ Antibodies

Various Adalimumab formulations were tested for the suitability to maintain Adalimumab physical and chemical stability under both accelerated stability test conditions and long-term storage at recommended storage temperature conditions (see Table 1 below). Formulations differed in pH (pH 5.2 vs. pH 6), excipient conditions (e.g., concentrations of mannitol or sorbitol), salt/ionic strength conditions (e.g., concentration of NaCl), and protein concentration (50 mg/mL vs. 100 mg/mL).

TABLE 24
Overview Of Formulations Referenced In The Following
Examples (All Concentrations Refer to mg/mL).

| Component | F1 | F2 | F3 | F4 | F5 | F6 | F7 |
|---|---|---|---|---|---|---|---|
| Adalimumab | 100 | 100 | 100 | 100 | 100 | 100 | 50 |
| mannitol | 12 | 42 | — | 12 | 42 | — | 12 |
| sorbitol | — | — | 42 | — | — | 42 | — |
| Polysorbate 80 | 1 | 1 | 1 | 1 | 1 | 1 | 1 |
| citric acid * H₂O | 1.305 | 1.305 | 1.305 | 1.305 | 1.305 | 1.305 | 1.305 |
| Sodium citrate | 0.305 | 0.305 | 0.305 | 0.305 | 0.305 | 0.305 | 0.305 |
| dihydrate | | | | | | | |
| Na₂HPO₄ * 2 H₂O | 1.53 | 1.53 | 1.53 | 1.53 | 1.53 | 1.53 | 1.53 |
| NaH₂PO₄ * 2 H₂O | 0.86 | 0.86 | 0.86 | 0.86 | 0.86 | 0.86 | 0.86 |
| NaCl | 6.165 | 0 | 0 | 6.165 | 0 | 0 | 6.165 |
| NaOH | q.s | q.s | q.s | q.s | q.s | q.s | q.s |
| target pH | 5.2 | 5.2 | 5.2 | 6.0 | 6.0 | 6.0 | 5.2 |

Table 2 provides an overview of stress temperatures and sample pull points. Formulations F2 and F6 were identified as formulations that maintain both the physical and chemical stability of Adalimumab for at least 18 months and 12 months, respectively. An exchange of the formulation excipient NaCl with mannitol (formulation F2) and sorbitol (formulation F6) conveys high stabilization potential, despite a 100% increase in protein concentration (from 50 mg/mL in formulation F7 to 100 mg/mL in formulations F2 and F6). Surprisingly, physical stability in both formulations were maintained for at least 12 and 18 months, respectively. Even after 12 months storage, both formulations contained more than 99% monomer (SEC data), and aggregate levels were below 1%.

Similarly, chemical stability, which very often is a shelf-life limiting factor in protein drug products, was maintained throughout the stability monitoring, since the stability indicating sum of lysine variants (L0+L1+L2) exceeded 80%.

Additional tests accepted in the art as being suitable to monitor physical and/or chemical stability of protein formulations confirmed the stabilization potential of formulations F2 and F6, e.g., subvisible particle testing, turbidity measurement, visual inspection, clarity or color monitoring.

As importantly, efficacy indicating anti-TNF neutralization testing showed that both formulations maintained efficacy of Adalimumab throughout the complete sample pull schedule, and data were within a high quality level range of 75 to 125%.

TABLE 25
Stability Data Obtained for F2 and F6 Formulations at
Various Temperatures for Various Months.

| | 5° C. | 25° C./60% R.H | 40° C./75% R.H. |
|---|---|---|---|
| F2 | 9 months | 6 months | 6 months |
| F6 | 3 months | 3 months | 3 months |
| F2 | 18 months | 6 months | 6 months |
| F6 | 12 months | 6 months | 6 months |

TABLE 26
Selected Stability Test Data Of Formulation F2 And Formulation F6 - Long-Term, Up To 9 Months.

| | | | F2 | | | F6 | | |
|---|---|---|---|---|---|---|---|---|
| | | | Storage Conditions | | | Storage Conditions | | |
| | | | [° C./% R.H.] | | | [° C./% R.H.] | | |
| | | Duration | | 25° C./ | 40° C./ | | 25° C./ | 40° C./ |
| Test Item | Component | of Testing | 5° C. | 60% R.H. | 75% R.H. | 5° C. | 60% R.H. | 75% R.H. |
| Particulate | Visual Score | Initial | 0.0 | 0.0 | 0.0 | 0 | 0 | 0 |
| Contamination: | | 3 Months | 0.0 | 0.0 | 0.0 | 0 | 0 | 0 |
| Visible Particles | | 6 Months | 0.0 | 0.0 | 0.0 | — | — | — |
| | | 9 Months | 0.0 | — | — | — | — | — |
| Clarity | Turbidity | Initial | 19.40 | 19.40 | 19.40 | 35.7 | 35.7 | 35.7 |
| | | 3 Months | 18.70 | 19.90 | 21.60 | 35.1 | 35 | 37 |
| | | 6 Months | 20.30 | 21.00 | 28.20 | — | — | — |
| | | 9 Months | 20.50 | — | — | — | — | — |
| | Blank | Initial | 0.08 | 0.08 | 0.08 | 0.31 | 0.31 | 0.31 |
| | | 3 Months | 0.15 | 0.34 | 0.21 | 0.28 | 0.16 | 0.29 |
| | | 6 Months | 0.15 | 0.46 | 0.22 | — | — | — |
| | | 9 Months | 0.08 | — | — | — | — | — |
| Color | B Scale | Initial | — | — | — | — | — | — |
| | | 3 Months | — | — | — | — | — | — |
| | | 6 Months | — | — | — | — | — | — |
| | | 9 Months | — | — | — | — | — | — |
| | BY Scale | Initial | <=BG 7 | <=BG 7 | <=BG 7 | <=BG 7 | <=BG 7 | <=BG 7 |
| | | 3 Months | <=BG 7 | <=BG 7 | <=BG 7 | <=BG 7 | <=BG 7 | <=BG 7 |
| | | 6 Months | <=BG 7 | <=BG 7 | <=BG 6 | — | — | — |
| | | 9 Months | <=BG 7 | — | — | — | — | — |
| pH | Single Value | Initial | 5.3 | 5.3 | 5.3 | 6 | 6 | 6 |
| | | 3 Months | 5.3 | 5.3 | 5.3 | 6 | 6 | 6 |
| | | 6 Months | 5.3 | 5.3 | 5.4 | — | — | — |
| | | 9 Months | 5.3 | — | — | — | — | — |
| Particulate | Particles >=1 | 3 Months | 3936 | 4522 | 6688 | 3203 | 3328 | 4834 |
| Contamination: | μm | 6 Months | 4372 | 4470 | 3788 | — | — | — |
| Subvisible Particles | | 9 Months | 19709 | — | — | — | — | — |
| | Particles >=10 | Initial | 17 | 17 | 17 | 15 | 15 | 15 |
| | μm [/Unit.] | 3 Months | 8 | 23 | 28 | 6 | 11 | 45 |
| | | 6 Months | 34 | 39 | 46 | — | — | — |
| | | 9 Months | 127 | — | — | — | — | — |
| | Particles >=25 | Initial | 0 | 0 | 0 | 0 | 0 | 0 |
| | μm [/Unit.] | 3 Months | 0 | 0 | 0 | 0 | 0 | 1 |
| | | 6 Months | 0 | 0 | 1 | — | — | — |
| | | 9 Months | 1 | — | — | — | — | — |
| Cation Exchange | 1st Acidic | Initial | 2.8 | 2.8 | 2.8 | 2.9 | 2.9 | 2.9 |
| HPLC (CEX-HPLC) | Region [%] | 3 Months | 2.8 | 6.9 | 36.1 | 2.7 | 5 | 22.2 |
| | | 6 Months | 2.9 | 11.3 | 58.0 | — | — | — |
| | | 9 Months | 3.1 | — | — | — | — | — |
| | 2nd Acidic | Initial | 10.7 | 10.7 | 10.7 | 10.9 | 10.9 | 10.9 |
| | Region [%] | 3 Months | 10.9 | 17.3 | 34.7 | 11 | 16.7 | 40 |
| | | 6 Months | 11.0 | 22.2 | 25.1 | — | — | — |
| | | 9 Months | 11.2 | — | — | — | — | — |
| | Sum Of Lysine | Initial | 84.2 | 84.2 | 84.2 | 84 | 84 | 84 |
| | Variants [%] | 3 Months | 84.2 | 72.3 | 24.6 | 84.7 | 75.7 | 33.2 |
| | | 6 Months | 83.9 | 61.7 | 10.9 | — | — | — |
| | | 9 Months | 83.2 | — | — | — | — | — |
| | Peaks After | Initial | 1.0 | 1.0 | 1.0 | 1.4 | 1.4 | 1.4 |
| | Lysine 2 [%] | 3 Months | 0.7 | 1.4 | 2.2 | 0.8 | 1.3 | 2.4 |
| | | 6 Months | 0.9 | 2.3 | 4.2 | — | — | — |
| | | 9 Months | 1.1 | — | — | — | — | — |
| | Peak Between | Initial | 1.3 | 1.3 | 1.3 | 0.8 | 0.8 | 0.8 |
| | Lysine 1 And | 3 Months | 1.4 | 2.1 | 2.5 | 0.8 | 1.4 | 2.3 |
| | Lysine 2 [%] | 6 Months | 1.4 | 2.4 | 1.9 | — | — | — |
| | | 9 Months | 1.4 | — | — | — | — | — |
| Size Exclusion | Principal Peak | Initial | 99.4 | 99.4 | 99.4 | 98.9 | 98.9 | 98.9 |
| Chromatography | (Monomer) [%] | 3 Months | 99.4 | 98.9 | 96.4 | 99 | 98.3 | 96 |
| (SE-HPLC) | | 6 Months | 99.4 | 98.5 | 93.2 | — | — | — |
| Adalimumab | | 9 Months | 99.3 | — | — | — | — | — |
| | Aggregate | Initial | 0.5 | 0.5 | 0.5 | 0.9 | 0.9 | 0.9 |
| | Average | 3 Months | 0.5 | 0.7 | 1.7 | 1 | 1.4 | 2.7 |
| | | 6 Months | 0.5 | 0.9 | 3.3 | — | — | — |
| | | 9 Months | 0.6 | — | — | — | — | — |
| | Fragment | Initial | 0.1 | 0.1 | 0.1 | 0.1 | 0.1 | 0.1 |
| | Average | 3 Months | 0.1 | 0.4 | 1.9 | 0.1 | 0.3 | 1.3 |
| | | 6 Months | 0.1 | 0.7 | 3.4 | — | — | — |
| | | 9 months | 0.1 | — | — | — | — | — |

TABLE 27
Selected Stability Test Data Of Formulation F2 And Formulation F6 - Long-Term, Up To 18 Months.

| | | | F2 | | | F6 | | |
|---|---|---|---|---|---|---|---|---|
| | | | E09807001CL | | | E09808001CL | | |
| | | | Storage Conditions | | | Storage Conditions | | |
| | | | [° C./% R.H.] | | | [° C./% R.H.] | | |
| | | Duration | | 25° C./ | 40° C./ | | 25° C./ | 40° C./ |
| Test Item | Component | of Testing | 5° C. | 60% R.H. | 75% R.H. | 5° C. | 60% R.H. | 75% R.H. |
| Particulate | Visual Score | Initial | 0 | 0 | 0 | 0 | 0 | 0 |
| Contamination: | | 3 Months | 0 | 0 | 0 | 0.2 | 0 | 0 |
| Visible Particles | | 6 Months | 0 | 0 | 0.2 | 0.1 | 0.1 | 0.2 |
| | | 9 Months | 0 | — | — | 0 | — | — |
| | | 12 Months | 0 | — | — | 0.2 | — | — |
| | | 18 Months | 0 | — | — | — | — | — |
| Clarity | Turbidity | Initial | 19.4 | 19.4 | 19.4 | 37.3 | 37.3 | 37.3 |
| | | 3 Months | 20.1 | 20.3 | 23 | 38.1 | 38.2 | 39.6 |
| | | 6 Months | 18.4 | 19.5 | 26.2 | 35.3 | 35.1 | 41.7 |
| | | 9 Months | 22.9 | — | — | 43 | — | — |
| | | 12 Months | 18.1 | — | — | 34.5 | — | — |
| | | 18 Months | 19.1 | — | — | — | — | — |
| | Blank | Initial | 0.16 | 0.16 | 0.16 | 0.09 | 0.09 | 0.09 |
| | | 3 Months | 0.13 | 0.15 | 0.06 | 0.06 | 0.19 | 0.19 |
| | | 6 Months | 0.05 | 0.08 | 0.04 | 0.05 | 0.03 | 0.02 |
| | | 9 Months | 0.06 | — | — | 0.18 | — | — |
| | | 12 Months | 0.09 | — | — | 0.09 | — | — |
| | | 18 Months | 0.11 | — | — | — | — | — |
| Degree Of Coloration | B Scale | Initial | =B 9 | =B 9 | =B 9 | =B 9 | =B 9 | =B 9 |
| Of Liquids | | 3 Months | <=B 7 | <=B 7 | <=B 7 | <=B 7 | <=B 7 | <=B 7 |
| | | 6 Months | <=B 8 | <=B 8 | <=B 7 | <=B 8 | <=B 7 | <=B 6 |
| | | 9 Months | <=B 7 | — | — | <=B 7 | — | — |
| | | 12 Months | <=B 7 | — | — | <=B 7 | — | — |
| | | 18 Months | <=B 7 | — | — | — | — | — |
| | BY Scale | Initial | — | — | — | — | — | — |
| | | 3 Months | — | — | — | — | — | — |
| | | 6 Months | <=BG 7 | <=BG 7 | <=BG 6 | <=BG 7 | <=BG 7 | <=BG 6 |
| | | 9 Months | <=BG 7 | — | — | <=BG 7 | — | — |
| | | 12 Months | <=BG 7 | — | — | <=BG 7 | — | — |
| | | 18 Months | <=BG 7 | — | — | — | — | — |
| pH | Single Value | Initial | 5.3 | 5.3 | 5.3 | 6.1 | 6.1 | 6.1 |
| | | 3 Months | 5.3 | 5.3 | 5.3 | 6.1 | 6.1 | 6.1 |
| | | 6 Months | 5.3 | 5.3 | 5.3 | 6.1 | 6.1 | 6.1 |
| | | 9 Months | 5.3 | — | — | 6.1 | — | — |
| | | 12 Months | 5.2 | — | — | 6.1 | — | — |
| | | 18 Months | 5.3 | — | — | — | — | — |
| Particulate | Particles >=1 | 9 Months | 4738 | — | — | 6177 | — | — |
| Contamination: | μm | 12 Months | 5329 | — | — | 5793 | — | — |
| Subvisible Particles | | 18 Months | 12589 | — | — | — | — | — |
| | Particles >=10 | Initial | 19 | 19 | 19 | 18 | 18 | 18 |
| | μm [/Unit] | 3 Months | 34 | 67 | 62 | 42 | 64 | 71 |
| | | 6 Months | 18 | 48 | 72 | 23 | 36 | 54 |
| | | 9 Months | 11 | — | — | 21 | — | — |
| | | 12 Months | 16 | — | — | 22 | — | — |
| | | 18 Months | 60 | — | — | — | — | — |
| | Particles >=25 | Initial | 0 | 0 | 0 | 0 | 0 | 0 |
| | μm [/Unit] | 3 Months | 0 | 1 | 2 | 0 | 1 | 2 |
| | | 6 Months | 0 | 1 | 2 | 0 | 0 | 0 |
| | | 9 Months | 0 | — | — | 0 | — | — |
| | | 12 Months | 0 | — | — | 0 | — | — |
| | | 18 Months | 0 | — | — | — | — | — |
| Cation Exchange | First Acidic | Initial | 2.2 | 2.2 | 2.2 | 2.1 | 2.1 | 2.1 |
| HPLC (CEX-HPLC) | Region Average | 3 Months | 2.2 | 6.3 | 35.8 | 2.0 | 3.8 | 21.6 |
| | [%] | 6 Months | 2.4 | 11.4 | 59.1 | 2.2 | 6.2 | 44.6 |
| | | 9 Months | 2.7 | — | — | 2.4 | — | — |
| | | 12 Months | 2.9 | — | — | 2.4 | — | — |
| | | 18 Months | 3.3 | — | — | — | — | — |
| | Second Acidic | Initial | 10.3 | 10.3 | 10.3 | 10.2 | 10.2 | 10.2 |
| | Region Average | 3 Months | 10.6 | 16.7 | 32.8 | 10.6 | 15.7 | 40.1 |
| | [%] | 6 Months | 10.8 | 22.4 | 22.0 | 10.6 | 21.2 | 32.0 |
| | | 9 Months | 11.1 | — | — | 10.9 | — | — |
| | | 12 Months | 11.3 | — | — | 11.0 | — | — |
| | | 18 Months | 11.9 | — | — | — | — | — |
| | L0 + L1 + L2 | Initial | 85.9 | 85.9 | 85.9 | 86.4 | 86.4 | 86.4 |
| | Average [%] | 3 Months | 85.0 | 73.4 | 22.5 | 86.0 | 78.2 | 30.1 |
| | | 6 Months | 84.8 | 62.0 | 9.6 | 85.8 | 69.8 | 12.9 |
| | | 9 Months | 84.0 | — | — | 85.3 | — | — |
| | | 12 Months | 83.6 | — | — | 85.0 | — | — |
| | | 18 Months | 82.4 | — | — | — | — | — |
| | Peaks After | Initial | 0.6 | 0.6 | 0.6 | 0.7 | 0.7 | 0.7 |
| | Lysine 2 [%] | 3 Months | 1.0 | 1.7 | 6.5 | 0.7 | 1.2 | 5.3 |
| | | 6 Months | 0.7 | 1.8 | 7.5 | 0.7 | 1.4 | 7.6 |
| | | 9 Months | 0.7 | — | — | 0.7 | — | — |
| | | 12 Months | 0.7 | — | — | 0.8 | — | — |
| | | 18 Months | 0.9 | — | — | — | — | — |
| | Peak Between | Initial | 1.1 | 1.1 | 1.1 | 0.6 | 0.6 | 0.6 |
| | Lysine 1 And | 3 Months | 1.2 | 2.0 | 2.4 | 0.6 | 1.1 | 2.9 |
| | Lysine 2 [%] | 6 Months | 1.4 | 2.4 | 1.8 | 0.7 | 1.5 | 2.9 |
| | | 9 Months | 1.4 | — | — | 0.8 | — | — |
| | | 12 Months | 1.5 | — | — | 0.8 | — | — |
| | | 18 Months | 1.5 | — | — | — | — | — |
| HPLC (SE-HPLC) | Principal Peak | Initial | 99.6 | 99.6 | 99.6 | 99.2 | 99.2 | 99.2 |
| Adalimumab | (Monomer) [%] | 3 Months | 99.1 | 98.6 | 96.0 | 99.2 | 98.7 | 96.5 |
| | | 6 Months | 99.0 | 98.0 | 91.9 | 99.1 | 98.2 | 91.5 |
| | | 9 Months | 99.5 | — | — | 99.1 | — | — |
| | | 12 Months | 99.5 | — | — | 99.1 | — | — |
| | | 18 Months | 99.4 | — | — | — | — | — |
| | Aggregate | Initial | 0.3 | 0.3 | 0.3 | 0.6 | 0.6 | 0.6 |
| | Average | 3 Months | 0.8 | 1.0 | 1.6 | 0.7 | 1.0 | 2.0 |
| | | 6 Months | 0.8 | 1.2 | 3.7 | 0.7 | 1.2 | 5.7 |
| | | 9 Months | 0.4 | — | — | 0.8 | — | — |
| | | 12 Months | 0.4 | — | — | 0.8 | — | — |
| | | 18 Months | 0.4 | — | — | — | — | — |
| | Fragments | Initial | 0.1 | 0.1 | 0.1 | 0.1 | 0.1 | 0.1 |
| | Average | 3 Months | 0.1 | 0.4 | 2.4 | 0.1 | 0.3 | 1.4 |
| | | 6 Months | 0.2 | 0.8 | 4.4 | 0.2 | 0.6 | 2.9 |
| | | 9 Months | 0.2 | — | — | 0.1 | — | — |
| | | 12 Months | 0.1 | — | — | 0.1 | — | — |
| | | 18 Months | 0.2 | — | — | — | — | — |
| Protein Content (UV 280 nm) | Mean [mg/mL] | Initial | 97.5 | 97.5 | 97.5 | 98.2 | 98.2 | 98.2 |
| Photon Correlation | PDI | Initial | 0.057 | 0.057 | 0.057 | 0.061 | 0.061 | 0.061 |
| Spectroscopy | Average | 3 Months | 0.063 | 0.062 | 0.126 | 0.058 | 0.057 | 0.083 |
| | | 6 Months | 0.058 | 0.063 | 0.234 | 0.062 | 0.145 | 0.217 |
| | | 9 Months | 0.059 | — | — | 0.058 | — | — |
| | | 12 Months | 0.063 | — | — | 0.059 | — | — |
| | | 18 Months | 0.057 | — | — | — | — | — |
| | Z Average | Initial | 4.8 | 4.8 | 4.8 | 7.1 | 7.1 | 7.1 |
| | Mean | 3 Months | 4.9 | 4.9 | 5.3 | 7.1 | 7.1 | 7.3 |
| | | 6 Months | 4.8 | 4.9 | 6.2 | 7.1 | 7.6 | 8.4 |
| | | 9 Months | 4.8 | — | — | 7.1 | — | — |
| | | 12 Months | 4.8 | — | — | 7.1 | — | — |
| | | 18 Months | 4.8 | — | — | — | — | — |
| In Vitro TNF- | Sample | Initial | 103 | 103 | 103 | 107 | 107 | 107 |
| Neutralization | [%] | 3 Months | 115 | 93 | 91 | 104 | 90 | 93 |
| (Cytotoxicity Test) | | 6 Months | 98 | 78 | 71 | 119 | 103 | 82 |
| | | 9 Months | 102 | — | — | 94 | — | — |
| | | 12 Months | 89 | — | — | 86 | — | — |

### Example 9

### Pain Study of High Concentration Adalimumab

Patients receiving monoclonal antibody treatment by subcutaneous injection may experience pain or discomfort at the injection site (see, e.g., Fransson, J.; Espander-Jansson, A. (1996) Journal of Pharmacy and Pharmacology 48(10), 1012-1015; Parham, S. M.; Pasieka, J. L. (1996) Can. J. Surg. 39, 31-35; Moriel E Z; Rajfer J (1993) The Journal of urology 149(5 Pt 2), 1299-300). An animal model that mimics the patient experience was used to assess pain and tolerability effects and to assess possible formulation modifications prior to human use. Available animal models were assessed for their suitability for differentiating characteristics of protein formulations. Measurements included vocalization on injection, paw flinching (at 0-10 minutes post injection), tests of mechanical allodynia, and thermal hyperalgesia (30 minutes post injection). Animals were also observed for nociceptive behaviors, such as licking or shaking the affected paw, and redness or swelling at injection site.

The flinching model was chosen to assess injection site pain, and was used to evaluate impact of formulation composition on tolerability and pain sensations.

Tolerability of various Adalimumab 100 mg/mL formulations were compared to formulation F7 (a 50 mg/mL Adalimumab formulation). The data generated supported the surprising findings of improved tolerability of the 100 mg/mL formulations at the injection site after subcutaneous injection as compared to 50 mg/mL formulations (F7).

The new 100 mg/mL formulations were optimized to reduce subcutaneous injection-related side effects such as pain at the injection site. Injection site pain comprises both pain related to the needle prick and sensations related to the infusion of the solution into the subQ depot. Whereas data available in the literature suggested that certain needle designs may be advantageous to reduce injection site discomfort, no clear data on the formulation contribution was available (see, e.g., Chan, G. C. F., et al. (2003) American Journal of Hematology 76(4):398-404).

Our data using a rat pain model suggested that the new 100 mg/mL formulations are effective in reducing injection site pain after subcutaneous injection of similar therapeutic doses as compared to the currently marketed Humira® formulation. This was achieved by reduced injection volume of the new 100 mg/mL formulations, showing a highly valuable benefit of optimizing patient treatments and increasing patient compliance.

At the same time, we observed that formulation pH in a range acceptable for formulating the 100 mg/mL formulation does not affect injection site pain. Interestingly, lower pH values that are further from the physiological pH range could be administered with similar tolerability.

### Method Applied for Tolerability Testing:

### Paw Flinching and Nocifensive Behavior Assays

Adult, male Sprague Dawley rats were acclimated to testing conditions for 20-30 minutes prior to intraplantar (s.c.) injection of test solutions into the right hind paw. The number of paw flinches was noted and the time spent in nocifensive behaviors (paw guarding or licking) was quantified for the first 10 minutes following injection. All test solutions were injected in a total volume of 150 μL unless otherwise noted. Experiments were coded and run in a blinded, randomized fashion. Saline and capsaicin (2.5 μg) were used as negative and positive controls, respectively.

### Volume Effect

The effect of injection volume on the paw flinching response was tested in both placebo and test formulation F7. To determine whether the response could be ameliorated by decreasing the physical volume, the effect of varying injection volumes (10 μl, 50 μl, and 150 μl intraplantar) on flinching outcomes was tested.

Test data allow for the following summary of volume effect: Flinching was significantly increased at 150 μl in both placebo (32±12) and F7 compared to saline (4±2), but not distinguishable from saline at smaller volumes. Whereas higher injection volume of 150 μL consistently produced higher flinching responses, the lower volume (10 μL and 50 μL) resulted in significantly lower responses.

This outcome suggests that reducing the volume of injectate is less irritating, suggesting that high concentration formulations, such as F2 and F6, are advantageous with regard to tolerability and pain sensation as compared to lower concentration formulations, such as F7.

Number of paw flinches 0-10 minutes post injection for placebo injections:

One-way ANOVA: 10; 50; 150 μL injection volume

| Source | DF | SS | MS | F | P |
|---|---|---|---|---|---|
| Factor | 2 | 2696 | 1348 | 4.32 | 0.033 |
| Error | 15 | 4679 | 312 | | |
| Total | 17 | 7376 | | | |

| | | |
|---|---|---|
| S = 17.66 | R-Sq = 36.56% | R-Sq (adj) = 28.10% |

| | | | | |
|---|---|---|---|---|
| | | | | Individual 95% CIs For Mean |
| | | | | Based on Pooled StDev |
| Level | N | Mean | StDev | -------+---------+---------+---------+-- |
| 10 | 6 | 4.17 | 4.79 | (---------*---------) |
| 50 | 6 | 7.17 | 9.54 | (---------*---------) |
| 150 | 6 | 31.50 | 28.67 | (---------*---------) |
| | | | | -------+---------+---------+---------+-- |
| | | | | 0 15 30 45 |

Number of paw flinches 0-10 min post injection for active injections (test formulation F7):

One-way ANOVA: 10; 50; 150 μL injection volume

| Source | DF | SS | MS | F | P |
|---|---|---|---|---|---|
| Factor | 2 | 4075 | 2037 | 6.96 | 0.007 |
| Error | 15 | 4390 | 293 | | |
| Total | 17 | 8465 | | | |

| | | |
|---|---|---|
| S = 17.11 | R-Sq = 48.14% | R-Sq (adj) = 41.22% |

| | | | | |
|---|---|---|---|---|
| | | | | Individual 95% CIs For Mean |
| | | | | Based on Pooled StDev |
| Level | N | Mean | StDev | ---+---------+---------+---------+------ |
| 10 | 6 | 9.67 | 9.11 | (--------*--------) |
| 50 | 6 | 9.50 | 8.12 | (--------*--------) |
| 150 | 6 | 41.50 | 27.00 | (--------*--------) |
| | | | | ---+---------+---------+---------+------ |
| | | | | 0 16 32 48 |

Pooled StDev = 17.11

### Example 10

### pH Effect of Adalimumab Containing Solutions on Tolerability/Pain

An additional experiment was carried out with adalimumab containing active solutions. Formulations tested were F2 (at pH 5.2), F5, and F7, the corresponding formulations at pH values closer to the physiological conditions.

The data suggested that pH did not seem to have an effect on the animal response as measured using the paw flinching response and time spent in nocifensive behaviors. Positive and negative control data were within the expected range. It is well documented in the literature that lower formulation pH (i.e., acidic) can increase the risk of intolerability and pain sensations upon parenteral administration, especially with subcutaneous injections. Thus, it was surprising that for the F2 and F5 Adalimumab formulations the formulation pH did not impact tolerability and/or pain sensation. This is highly beneficial, since this allows other parameters, such as formulation pH, physical stability and aggregate levels (being potentially correlated to immunogenicity risks), a high priority with regard to formulation decision making.

Time spent in nocifensive behavior [sec] data:

One-way ANOVA: neg control; F2; F5; F8; pos control

| Source | DF | SS | MS | F | P |
|---|---|---|---|---|---|
| Factor | 4 | 919856 | 229964 | 27.81 | 0.000 |
| Error | 55 | 454773 | 8269 | | |
| Total | 59 | 1374629 | | | |

| | | |
|---|---|---|
| S = 90.93 | R-Sq = 66.92% | R-Sq (adj) = 64.51% |

| | | | | |
|---|---|---|---|---|
| | | | | Individual 95% CIs For Mean Based on |
| | | | | Pooled StDev |
| Level | N | Mean | StDev | ----+---------+---------+---------+----- |
| neg control | 12 | 0.00 | 0.00 | (---*---) |
| F2 | 12 | 177.75 | 82.84 | (---*--) |
| F5 | 12 | 215.25 | 91.52 | (--*---) |
| F8 | 12 | 129.58 | 83.13 | (---*--) |
| pos control | 12 | 381.17 | 138.54 | (--*---) |
| | | | | ----+---------+---------+---------+----- |
| | | | | 0 150 300 450 |

Pooled StDev = 90.93

Number of paw flinches 0-10 min post injection:

One-way ANOVA: Sal; F2; F5; F8; Cap

| Source | DF | SS | MS | F | P |
|---|---|---|---|---|---|
| Factor | 4 | 91404 | 22851 | 49.81 | 0.000 |
| Error | 55 | 25234 | 459 | | |
| Total | 59 | 116638 | | | |

| | | |
|---|---|---|
| S = 21.42 | R-Sq = 78.37% | R-Sq (adj) = 76.79% |

| | | | | |
|---|---|---|---|---|
| | | | | Individual 95% CIs For Mean |
| | | | | Based on Pooled StDev |
| Level | N | Mean | StDev | --+---------+---------+---------+------- |
| Sal | 12 | 4.92 | 3.18 | (--*--) |
| F2 | 12 | 71.00 | 14.84 | (--*--) |
| F5 | 12 | 93.67 | 24.07 | (--*---) |
| F8 | 12 | 58.83 | 25.23 | (--*--) |
| Cap | 12 | 121.92 | 29.12 | (--*---) |
| | | | | --+---------+---------+---------+------- |
| | | | | 0 40 80 120 |

Pooled StDev = 21.42

Tukey 95% Simultaneous Confidence Intervals

### Example 11

### Impact of Formulation pH Effect of Adalimumab Free Solutions

In order to test the impact of the formulation composition (e.g., the impact of buffers such as phosphate, excipients such as mannitol, or surfactants such as Polysorbate 80), an additional experiment was conducted where similar data were obtained with protein free formulations. The pH of the placebo solutions varied in a range of about 5-7 and surprisingly did not seem to have the effect of ameliorating pain, as the flinching response noted for formulations with different pH were similar. As explained earlier, this is highly beneficial in biologics drug product formulation development, since this allows formulators to give other parameters such as formulation pH, physical stability and aggregate levels (being potentially correlated to immunogenicity risks) a high priority with regard to formulation decision making.

Number of paw flinches 0-10 minutes post injection for placebo injections:

One-way ANOVA: Sal; 5.2; 6; 7; Cap

| Source | DF | SS | MS | F | P |
|---|---|---|---|---|---|
| Factor | 4 | 12241 | 3060 | 12.41 | 0.000 |
| Error | 20 | 4933 | 247 | | |
| Total | 24 | 17174 | | | |

| | | |
|---|---|---|
| S = 15.70 | R-Sq = 71.28% | R-Sq (adj) = 65.53% |

| | | | | |
|---|---|---|---|---|
| | | | | Individual 95% CIs For Mean |
| | | | | Based onPooled StDev |
| Level | N | Mean | StDev | -----+---------+---------+---------+---- |
| Sal | 5 | 2.00 | 1.41 | (-----*-----) |
| 5.2 | 5 | 49.00 | 26.47 | (-----*----) |
| 6 | 5 | 53.00 | 12.63 | (-----*-----) |
| 7 | 5 | 40.20 | 15.39 | (-----*-----) |
| Cap | 5 | 68.00 | 11.60 | (-----*-----) |
| | | | | -----+---------+---------+---------+---- |
| | | | | 0 25 50 75 |

Pooled StDev = 15.70

In summary, the data presented above clearly demonstrates the advantages of the 100 mg/mL Adalimumab formulations in that these high protein concentration, viscous solutions can be administered in lower volumes across a range of pHs without diminishing tolerability and/or increasing pain sensations.

### INCORPORATION BY REFERENCE

The contents of all cited references (including, for example, literature references, patents, patent applications, and websites) that maybe cited throughout this application are hereby expressly incorporated by reference in their entirety for any purpose. The practice of the present invention will employ, unless otherwise indicated, conventional techniques of protein formulations, which are well known in the art.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

What is claimed:

1. A liquid pharmaceutical formulation comprising:
a) 40-60 mg/mL of a recombinant human anti-hTNF-alpha antibody comprising a kappa light chain comprising complementarity determining regions having the amino acid sequences set forth as SEQ ID NO:3, SEQ ID NO:5, and SEQ ID NO:7; and an IgG1 heavy chain comprising complementarity determining regions having the amino acid sequences set forth as SEQ ID NO:4, SEQ ID NO:6, and SEQ ID NO:8; and
b) more than 20 mg/mL of a polyol;
wherein the formulation is essentially free of NaCl.

2. The formulation of claim 1, wherein the polyol is mannitol.

3. The formulation of claim 2, wherein the polyol is 40 to 80 mg/mL of mannitol.

4. The formulation of claim 1, wherein the polyol is sorbitol.

5. The formulation of claim 4, wherein the polyol is 50 to 100 mg/mL of sorbitol.

6. The formulation of claim 1, wherein the polyol is trehalose.

7. The formulation of claim 6, wherein the polyol is more than 40 mg/mL of trehalose.

8. The formulation of claim 1, wherein the formulation does not contain NaCl.

9. The formulation of claim 1, wherein the formulation comprises a surfactant.

10. The formulation of claim 9, wherein the surfactant is polysorbate 80.

11. The formulation of claim 10, wherein the surfactant is 0.1 to 2 mg/mL of polysorbate 80.

12. The formulation of claim 1, wherein the formulation comprises a buffer.

13. The formulation of claim 12, wherein the buffer is succinate.

14. The formulation of claim 12, wherein the buffer is histidine.

15. The formulation of claim 12, wherein the buffer is acetate.

16. The formulation of claim 12, wherein the formulation has a pH of 5.0 to 6.4.

17. The formulation of claim 1, which is suitable for subcutaneous administration.

18. The formulation of claim 1, wherein the antibody comprises a light chain variable region comprising the amino acid sequence set forth as SEQ ID NO:1 and a heavy chain variable region comprising the amino acid sequence set forth as SEQ ID NO:2.

19. The formulation of claim 18, wherein the antibody is adalimumab.

20. The formulation of claim 1, wherein the antibody is 50 mg/mL of a recombinant human anti-hTNF-alpha antibody comprising a kappa light chain comprising complementarity determining regions having the amino acid sequences set forth as SEQ ID NO:3, SEQ ID NO:5, and SEQ ID NO:7; and an IgG1 heavy chain comprising complementarity determining regions having the amino acid sequences set forth as SEQ ID NO:4, SEQ ID NO:6, and SEQ ID NO:8.

21. The formulation of claim 20, wherein the polyol is 40 to 80 mg/mL of mannitol.

22. The formulation of claim 21, wherein the formulation comprises 0.1 to 2 mg/mL of polysorbate 80.

23. The formulation of claim 22, wherein the formulation comprises histidine.

24. The formulation of claim 23, wherein the formulation has a pH of 5.8 to 6.4.

25. The formulation of claim 20, wherein the polyol is more than 40 mg/mL of trehalose.

26. The formulation of claim 25, wherein the formulation comprises 0.1 to 2 mg/mL of polysorbate 80.

27. The formulation of claim 26, wherein the formulation comprises succinate.

28. The formulation of claim 27, wherein the formulation has a pH of 5.8 to 6.4.

29. The formulation of claim 26, wherein the formulation comprises acetate.

30. The formulation of claim 29, wherein the formulation has a pH of 5.8 to 6.4.


SEQUENCE LISTING (37 sequences)

SEQ ID NO: 1 - protein, 107 aa, Artificial Sequence [sequence omitted]

SEQ ID NO: 2 - protein, 121 aa, Artificial Sequence [sequence omitted]

SEQ ID NO: 3 - protein, 9 aa, Artificial Sequence
  adalimumab light chain variable region CDR3
QRYNRAPYX

SEQ ID NO: 4 - protein, 12 aa, Artificial Sequence
  adalimumab heavy chain variable region CDR3
VSYLSTASSLDX

SEQ ID NO: 5 - protein, 7 aa, Artificial Sequence
  adalimumab light chain variable region CDR2
AASTLQS

SEQ ID NO: 6 - protein, 17 aa, Artificial Sequence
  adalimumab heavy chain variable region CDR2
AITWNSGHIDYADSVEG

SEQ ID NO: 7 - protein, 11 aa, Artificial Sequence
  adalimumab light chain variable region CDR1
RASQGIRNYLA

SEQ ID NO: 8 - protein, 5 aa, Artificial Sequence
  adalimumab heavy chain variable region CDR1
DYAMH

SEQ ID NO: 9 - protein, 107 aa, Artificial Sequence
  2SD4 light chain variable region
DIQMTQSPSSLSASIGDRVTITCRASQGIRNYLAWYQQKPGKAPKLLIYAASTLQSGVPSRFSGSGSGTDFTLTISSLQPEDVATYYCQKYNSAPYAFGQGTKVEIK

SEQ ID NO: 10 - protein, 121 aa, Artificial Sequence
  2SD4 heavy chain variable region
QVQLVESGGGLVQPGRSLRLSCAASGFTFDDYAMHWVRQAPGKGLDWVSAITWNSGHIDYADSVEGRFAVSRDNAKNALYLQMNSLRPEDTAVYYCTKASYLSTSSSLDNWGQGTLVTVSS

SEQ ID NO: 11 - protein, 9 aa, Artificial Sequence
  2SD4 light chain variable region CDR3
QKYNSAPYA

SEQ ID NO: 12 - protein, 9 aa, Artificial Sequence
  EP B12  light chain variable region CDR3
QKYNRAPYA

SEQ ID NO: 13 - protein, 9 aa, Artificial Sequence
  VL10E4 light chain variable region CDR3
QKYQRAPYT

SEQ ID NO: 14 - protein, 9 aa, Artificial Sequence
  VL100A9 light chain variable region CDR3
QKYSSAPYT

SEQ ID NO: 15 - protein, 9 aa, Artificial Sequence
  VLL100D2 light chain variable region CDR3
QKYNSAPYT

SEQ ID NO: 16 - protein, 9 aa, Artificial Sequence
  VLL0F4 light chain variable region CDR3
QKYNRAPYT

SEQ ID NO: 17 - protein, 9 aa, Artificial Sequence
  LOE5 light chain variable region CDR3
QKYNSAPYY

SEQ ID NO: 18 - protein, 9 aa, Artificial Sequence
  VLLOG7 light chain variable region CDR3
QKYNSAPYN

SEQ ID NO: 19 - protein, 9 aa, Artificial Sequence
  VLLOG9 light chain variable region CDR3
QKYTSAPYT

SEQ ID NO: 20 - protein, 9 aa, Artificial Sequence
  VLLOH1 light chain variable region CDR3
QKYNRAPYN

SEQ ID NO: 21 - protein, 9 aa, Artificial Sequence
  VLLOH10 light chain variable region CDR3
QKYNSAAYS

SEQ ID NO: 22 - protein, 9 aa, Artificial Sequence
  VL1B7 light chain variable region CDR3
QQYNSAPDT

SEQ ID NO: 23 - protein, 9 aa, Artificial Sequence
  VL1C1 light chain variable region CDR3
QKYNSDPYT

SEQ ID NO: 24 - protein, 9 aa, Artificial Sequence
  VL0.1F4 light chain variable region CDR3
QKYISAPYT

SEQ ID NO: 25 - protein, 9 aa, Artificial Sequence
  VL0.1H8 light chain variable region CDR3
QKYNRPPYT

SEQ ID NO: 26 - protein, 9 aa, Artificial Sequence
  LOE7.A light chain variable region CDR3
QRYNRAPYA

SEQ ID NO: 27 - protein, 12 aa, Artificial Sequence
  2SD4 heavy chain variable region CDR3
ASYLSTSSSLDN

SEQ ID NO: 28 - protein, 12 aa, Artificial Sequence
  VH1B11 heavy chain variable region CDR3
ASYLSTSSSLDK

SEQ ID NO: 29 - protein, 12 aa, Artificial Sequence
  VH1D8 heavy chain variable region CDR3
ASYLSTSSSLDY

SEQ ID NO: 30 - protein, 12 aa, Artificial Sequence
  VH1A11 heavy chain variable region CDR3
ASYLSTSSSLDD

SEQ ID NO: 31 - protein, 12 aa, Artificial Sequence
  VH1B12 heavy chain variable region CDR3
ASYLSTSFSLDY

SEQ ID NO: 32 - protein, 12 aa, Artificial Sequence
  VH1E4 heavy chain variable region CDR3
ASYLSTSSSLHY

SEQ ID NO: 33 - protein, 12 aa, Artificial Sequence
  VH1F6 heavy chain variable region CDR3
ASFLSTSSSLEY

SEQ ID NO: 34 - protein, 12 aa, Artificial Sequence
  3C-H2 heavy chain variable region CDR3
ASYLSTASSLEY

SEQ ID NO: 35 - protein, 12 aa, Artificial Sequence
  VH1-D2.N heavy chain variable region CDR3
VSYLSTASSLDN

SEQ ID NO: 36 - DNA, 321 bp, Artificial Sequence
  adalimumab light chain variable region
gacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 37 - DNA, 363 bp, Artificial Sequence
  adalimumab heavy chain variable region
gaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagt